(19) United States
(12) Patent Application Publication
(10) Pub. No.: US 2017/0334891 A1
(43) Pub. Date: Nov. 23, 2017

(54) ENHANCER OF ZESTE HOMOLOG 2 INHIBITORS

(71) Applicant: GLAXOSMITHKLINE INTELLECTUAL PROPERTY (NO.2) LIMITED, Brentford, Middlesex (GB)

(72) Inventors: Steven David KNIGHT, Collegeville, PA (US); Kenneth Allen NEWLANDER, Collegeville, PA (US); Xinrong TIAN, Collegeville, PA (US)

(73) Assignee: GlaxoSmithKline Intellectual Property (No.2) Limited, Brentford, Middlesex (GB)

(21) Appl. No.: 15/522,373

(22) PCT Filed: Oct. 28, 2015
(86) PCT No.: PCT/EP2015/075009

§ 371 Date: Apr. 27, 2017

Related U.S. Application Data
(60) Provisional application No. 62/069,438, filed on Oct. 28, 2014.
(60) Provisional application No. 62/075,596, filed on Nov. 5, 2014.

(51) Int. Cl. C07D 409/14; C07D 409/12; C07D 413/14; C07D 495/06

(52) U.S. Cl. CPC C07D 409/14; C07D 495/06; C07D 409/12; C07D 413/14

(57) ABSTRACT

This invention relates to novel compounds according to Formula (I) which are inhibitors of Enhancer of Zeste Homolog 2 (EZH2), to pharmaceutical compositions containing them, to processes for their preparation, and to their use in therapy for the treatment of cancers.

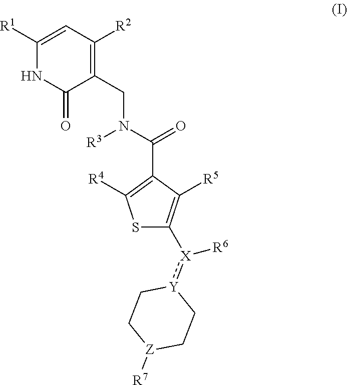

### FIELD OF THE INVENTION

This invention relates to compounds which inhibit Enhancer of Zeste Homolog 2 (EZH2) and thus are useful for inhibiting the proliferation of and/or inducing apoptosis in cancer cells.

### BACKGROUND OF THE INVENTION

Epigenetic modifications play an important role in the regulation of many cellular processes including cell proliferation, differentiation, and cell survival. Global epigenetic modifications are common in cancer, and include global changes in DNA and/or histone methylation, dysregulation of non-coding RNAs and nucleosome remodeling leading to aberrant activation or inactivation of oncogenes, tumor suppressors and signaling pathways. However, unlike genetic mutations which arise in cancer, these epigenetic changes can be reversed through selective inhibition of the enzymes involved. Several methylases involved in histone or DNA methylation are known to be dysregulated in cancer. Thus, selective inhibitors of particular methylases will be useful in the treatment of proliferative diseases such as cancer.

EZH2 (human EZH2 gene: Cardoso, C, et al; European J of Human Genetics, Vol. 8, No. 3 Pages 174-180, 2000) is the catalytic subunit of the Polycomb Repressor Complex 2 (PRC2) which functions to silence target genes by tri-methylating lysine 27 of histone H3 (H3K27me3). Histone H3 is one of the five main histone proteins involved in the structure of chromatin in eukaryotic cells. Featuring a main globular domain and a long N-terminal tail, Histones are involved with the structure of the nucleosomes, a ‘beads on a string’ structure. Histone proteins are highly post-translationally modified however Histone H3 is the most extensively modified of the five histones. The term “Histone H3” alone is purposely ambiguous in that it does not distinguish between sequence variants or modification state. Histone H3 is an important protein in the emerging field of epigenetics, where its sequence variants and variable modification states are thought to play a role in the dynamic and long term regulation of genes.

Increased EZH2 expression has been observed in numerous solid tumors including those of the prostate, breast, skin, bladder, liver, pancreas, head and neck and correlates with cancer aggressiveness, metastasis and poor outcome (Varambally et al., 2002; Kleer et al., 2003; Breuer et al., 2004; Bachmann et al., 2005; Weikert et al., 2005; Sudo et al., 2005; Bachmann et al., 2006). For instance, there is a greater risk of recurrence after prostatectomy in tumors expressing high levels of EZH2, increased metastasis, shorter disease-free survival and increased death in breast cancer patients with high EZH2 levels (Varambally et al., 2002; Kleer et al., 2003). More recently, inactivating mutations in UTX (ubiquitously transcribed tetratricopeptide repeats X), a H3K27 demethylase which functions in opposition to EZH2, have been identified in multiple solid and hematological tumor types (including renal, glioblastoma, esophageal, breast, colon, non-small cell lung, small cell lung, bladder, multiple myeloma, and chronic myeloid leukemia tumors), and low UTX levels correlate with poor survival in breast cancer suggesting that loss of UTX function leads to increased H3K27me3 and repression of target genes (Wang et al., 2010). Together, these data suggest that increased H3K27me3 levels contribute to cancer aggressiveness in many tumor types and that inhibition of EZH2 activity may provide therapeutic benefit.

Numerous studies have reported that direct knockdown of EZH2 via siRNA or shRNA or indirect loss of EZH2 via treatment with the SAH hydrolase inhibitor 3-deazaneplanocin A (DZNep) decreases cancer cell line proliferation and invasion in vitro and tumor growth in vivo (Gonzalez et al., 2008, GBM 2009). While the precise mechanism by which aberrant EZH2 activity leads to cancer progression is not known, many EZH2 target genes are tumor suppressors suggesting that loss of tumor suppressor function is a key mechanism. In addition, EZH2 overexpression in immortalized or primary epithelial cells promotes anchorage independent growth and invasion and requires EZH2 catalytic activity (Kleer et al., 2003; Cao et al., 2008).

Thus, there is strong evidence to suggest that inhibition of EZH2 activity decreases cellular proliferation and invasion. Accordingly, compounds that inhibit EZH2 activity would be useful for the treatment of cancer.

### SUMMARY OF THE INVENTION

The present invention relates to compounds according to Formula (I) or pharmaceutically acceptable salts thereof:

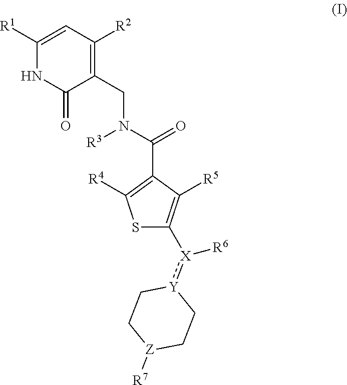

wherein:

X and Y are each independently CH, C, or N; wherein
when X is N, Y is CH, and [symbol] is a single bond;
when Y is N, X is CH, and [symbol] is a single bond;
when X and Y are each CH, [symbol] is a single bond; and
when X is C, Y is C, and [symbol] is a double bond;

Z is CH or N;

R¹ and R² are each independently (C₁-C₄)alkyl;

R³ and R⁴ are each hydrogen;

or R³ and R⁴ taken together represent —CH₂CH₂—;

R⁵ and R⁶ are each independently (C₁-C₃)alkyl; and

R⁷ is selected from the group consisting of halo(C₁-C₄)alkyl, —N((C₁-C₄)alkyl)₂, hydroxyl, pyrimidinyl, oxazolylmethyl, and —C(═N—CN)NH(C₁-C₄)alkyl;

provided that the compound is not N-((4,6-dimethyl-2-oxo-1,2-dihydropyridin-3-yl)methyl)-5-((4-(dimethylamino)cyclohexyl)(ethyl)amino)-4-methylthiophene-3-carboxamide, 5-((4-(dimethylamino)cyclohexyl)(ethyl)amino)-4-methyl-N-((6-methyl-2-oxo-4-propyl-1,2-dihydropyridin-3-yl)methyl)thiophene-3-carboxamide, N-((4,6-dimethyl-2-oxo-1,2-dihydropyridin-3-yl)methyl)-5-(1-(4-(dimethylamino)piperidin-1-yl)ethyl)-4-methylthiophene-3-carboxamide, N-((4,6-dimethyl-2-oxo-1,2-dihydropyridin-3-yl)methyl)-5-(1-(4-(dimethylamino)piperidin-1-yl)propyl)-4-methylthiophene-3-carboxamide, N-((4,6-dimethyl-2-oxo-1,2-dihydropyridin-3-yl)methyl)-5-(1-(4-(dimethylamino)cyclohexyl)propyl)-4-methylthiophene-3-carboxamide, or N-((4,6-dimethyl-2-oxo-1,2-dihydropyridin-3-yl)methyl)-5-(ethyl(4-(ethyl(methyl)amino)cyclohexyl)amino)-4-methylthiophene-3-carboxamide, or stereoisomers or mixtures thereof of each of these compounds.

Another aspect of this invention relates to a method of inducing apoptosis in cancer cells of solid tumors; treating solid tumor cancers.

Another aspect of the invention relates to pharmaceutical preparations comprising compounds of Formula (I) and pharmaceutically acceptable excipients.

In another aspect, there is provided the use of a compound of Formula (I) or a pharmaceutically acceptable salt or solvate thereof, in the preparation of a medicament for use in the treatment of a disorder mediated by EZH2, such as by inducing apoptosis in cancer cells.

In another aspect, this invention provides for the use of a compound of Formula (I) or a pharmaceutically acceptable salt thereof for the treatment of diseases mediated by EZH2. The invention further provides for the use of a compound of Formula (I) or a pharmaceutically acceptable salt thereof as an active therapeutic substance in the treatment of a disease mediated by EZH2.

In another aspect, the invention provides a compound of Formula (I) or a pharmaceutically acceptable salt thereof for use in therapy.

In another aspect, there is provided a compound of Formula (I) or a pharmaceutically acceptable salt thereof for use in the treatment of a disorder mediated by EZH2.

In another aspect, there is provided a compound of Formula (I) or a pharmaceutically acceptable salt thereof for use in the treatment of cellular proliferation diseases.

In another aspect, there is provided a compound of Formula (I) or a pharmaceutically acceptable salt thereof for use in the treatment of cancer, including the treatment of solid tumors, for example brain (gliomas), glioblastomas, leukemias, lymphomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, gastric, bladder, head and neck, kidney, lung, liver, melanoma, renal, ovarian, pancreatic, prostate, sarcoma, osteosarcoma, giant cell tumor of bone, and thyroid.

In another aspect there is provided methods of co-administering the presently invented compounds of Formula (I) with other active ingredients.

In another aspect there is provided a combination of a compound of Formula (I) or a pharmaceutically acceptable salt thereof and at least one anti-neoplastic agent for use in the treatment of a disorder mediated by EZH2.

In another aspect there is provided a combination of a compound of Formula (I) or a pharmaceutically acceptable salt thereof and at least one anti-neoplastic agent for use in the treatment of cellular proliferation diseases.

In another aspect there is provided a combination of a compound of Formula (I) or a pharmaceutically acceptable salt thereof and at least one anti-neoplastic agent for use in the treatment of cancer, including the treatment of solid tumors, for example brain (gliomas), glioblastomas, leukemias, lymphomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, gastric, bladder, head and neck, kidney, lung, liver, melanoma, renal, ovarian, pancreatic, prostate, sarcoma, osteosarcoma, giant cell tumor of bone, and thyroid.

### DETAILED DESCRIPTION OF THE INVENTION

This invention relates to compounds of the Formula (I) as defined above.

In one embodiment, this invention relates to compounds of Formula (I), wherein X and Y are each independently CH or N, wherein at least one of X and Y is CH and [symbol] is a single bond. In another embodiment, this invention relates to compounds of Formula (I), wherein X is N, Y is CH, and [symbol] is a single bond. In another embodiment, this invention relates to compounds of Formula (I), wherein Y is N, X is CH, and [symbol] is a single bond. In another embodiment, this invention relates to compounds of Formula (I), wherein X and Y are each CH and [symbol] is a single bond. In another embodiment, this invention relates to compounds of Formula (I), wherein X and Y are each C and [symbol] is a double bond.

In another embodiment, this invention relates to compounds of Formula (I), wherein X is CH or C and Y is CH, C, or N, wherein when Y is N, X is CH, and [symbol] is a single bond, when X and Y are each CH, [symbol] is a single bond, and when X is C, Y is C, and [symbol] is a double bond.

In a specific embodiment, this invention relates to compounds of Formula (I), wherein Z is CH. In another specific embodiment, this invention relates to compounds of Formula (I), wherein Z is N.

In another embodiment, this invention relates to compounds of Formula (I), wherein R¹ and R² are each independently methyl, ethyl, n-propyl, or n-butyl. In a specific embodiment, this invention relates to compounds of Formula (I), wherein R¹ and R² are each methyl.

In another specific embodiment, this invention relates to compounds of Formula (I), wherein R³ and R⁴ are each hydrogen. In another specific embodiment, this invention relates to compounds of Formula (I), wherein R³ and R⁴ taken together represent —CH₂CH₂—;

In another embodiment, this invention relates to compounds of Formula (I), wherein R⁵ and R⁶ are each independently methyl, ethyl, n-propyl, or isopropyl. In a specific embodiment, this invention relates to compounds of Formula (I), wherein R⁵ is methyl. In another specific embodiment, this invention relates to compounds of Formula (I), wherein R⁶ is ethyl.

In another embodiment, this invention relates to compounds of Formula (I), wherein R⁷ is selected from the group consisting of halo(C₁-C₄)alkyl, —N((C₁-C₄)alkyl)₂, and hydroxyl. In another embodiment, this invention relates to compounds of Formula (I), wherein R⁷ is halo(C₁-C₄)alkyl. In another embodiment, this invention relates to compounds of Formula (I), wherein R⁷ is —N((C₁-C₄)alkyl)₂. In a specific embodiment, this invention relates to compounds of Formula (I), wherein R⁷ is dimethylamino. In another specific embodiment, this invention relates to compounds of Formula (I), wherein R⁷ is hydroxyl.

In another embodiment, this invention relates to compounds of Formula (I), wherein R⁷ is —C(═N—CN)NH(C₁-C₄)alkyl. In another embodiment, this invention relates to compounds of Formula (I), wherein Z is N and R⁷ is —C(═N—CN)NH(C₁-C₄)alkyl. In a specific embodiment, this invention relates to compounds of Formula (I), wherein Z is N and R⁷ is —C(═N—CN)NHCH₃.

In another specific embodiment, this invention relates to compounds of Formula (I), wherein R⁷ is selected from the group consisting of 2-fluoropropyl, 2-fluoro-2-methylpropyl, 2,2-difluoroethyl, 2,2-difluoropropyl, 2,2,2-trifluoroethyl, hydroxyl, dimethylamino, pyrimidin-2-yl, oxazol-2-ylmethyl, and —C(═N—CN)NHCH₃. In another specific embodiment, this invention relates to compounds of Formula (I), wherein R⁷ is selected from the group consisting of 2-fluoro-2-methylpropyl, 2,2-difluoropropyl, 2,2,2-trifluoroethyl, hydroxyl, dimethylamino, pyrimidin-2-yl, and oxazol-2-ylmethyl. In another specific embodiment, this invention relates to compounds of Formula (I), wherein R⁷ is selected from the group consisting of 2-fluoro-2-methylpropyl, 2,2-difluoropropyl, and 2,2,2-trifluoroethyl. In a further specific embodiment, this invention relates to compounds of Formula (I), wherein R⁷ is 2-fluoro-2-methylpropyl. In yet a further specific embodiment, this invention relates to compounds of Formula (I), wherein R⁷ is 2,2-difluoropropyl.

In a particular embodiment, this invention relates to compounds of Formula (I), wherein when R⁷ is —N((C₁-C₄)alkyl)₂, at least one of the following conditions must be met:

(i) Z is N;

(ii) R³ and R⁴ taken together represent —CH₂CH₂—; or

(iii) X and Y are each C and [symbol] is a double bond.

In another particular embodiment, this invention relates to compounds of Formula (I), wherein when R⁷ is —N((C₁-C₄)alkyl)₂, Z is N. In another particular embodiment, this invention relates to compounds of Formula (I), wherein when R⁷ is —N((C₁-C₄)alkyl)₂, R³ and R⁴ taken together represent —CH₂CH₂—. In another particular embodiment, this invention relates to compounds of Formula (I), wherein when R⁷ is —N((C₁-C₄)alkyl)₂, X and Y are each C and [symbol] is a double bond.

In another embodiment, this invention also relates to compounds of Formula (Ia):

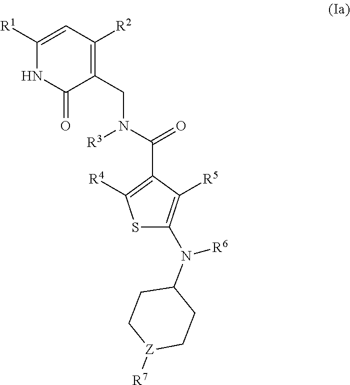

or pharmaceutically acceptable salts thereof, wherein Z, R¹, R², R³, R⁴, R⁵, R⁶, and R⁷ are defined according to Formula (I), provided that the compound is not N-((4,6-dimethyl-2-oxo-1,2-dihydropyridin-3-yl)methyl)-5-((4-(dimethylamino)cyclohexyl)(ethyl)amino)-4-methylthiophene-3-carboxamide, 5-((4-(dimethylamino)cyclohexyl)(ethyl)amino)-4-methyl-N-((6-methyl-2-oxo-4-propyl-1,2-dihydropyridin-3-yl)methyl)thiophene-3-carboxamide, or N-((4,6-dimethyl-2-oxo-1,2-dihydropyridin-3-yl)methyl)-5-(ethyl(4-(ethyl(methyl)amino)cyclohexyl)amino)-4-methylthiophene-3-carboxamide, or stereoisomers or mixtures thereof of each of these compounds.

In a particular embodiment, this invention relates to compounds of Formula (Ia), wherein:

Z is N;

R¹ and R² are each independently methyl, ethyl, n-propyl, or n-butyl;

R³ and R⁴ are each hydrogen;

or R³ and R⁴ taken together represent —CH₂CH₂—;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, hydroxyl, and —C(═N—CN)NH(C₁-C₄)alkyl;

or pharmaceutically acceptable salts thereof.

In another particular embodiment, this invention relates to compounds of Formula (Ia), wherein:

Z is N;

R¹ and R² are each independently methyl, ethyl, n-propyl, or n-butyl;

R³ and R⁴ are each hydrogen;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, and hydroxyl;

or pharmaceutically acceptable salts thereof.

In another particular embodiment, this invention relates to compounds of Formula (Ia), wherein:

Z is CH or N;

R¹ and R² are each independently methyl, ethyl, n-propyl, or n-butyl;

R³ and R⁴ taken together represent —CH₂CH₂—;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, and hydroxyl;

or pharmaceutically acceptable salts thereof.

In another embodiment, this invention also relates to compounds of Formula (Ib):

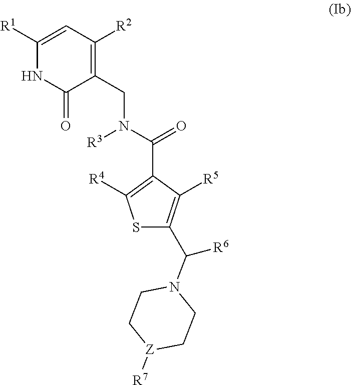

or pharmaceutically acceptable salts thereof, wherein Z, R¹, R², R³, R⁴, R⁵, R⁶, and R⁷ are defined according to Formula (I), provided that the compound is not N-((4,6-dimethyl-2-oxo-1,2-dihydropyridin-3-yl)methyl)-5-(1-(4-(dimethylamino)piperidin-1-yl)ethyl)-4-methylthiophene-3-carboxamide or N-((4,6-dimethyl-2-oxo-1,2-dihydropyridin-3-yl)methyl)-5-(1-(4-(dimethylamino)piperidin-1-yl)propyl)-4-methylthiophene-3-carboxamide, or stereoisomers or mixtures thereof of either of these compounds.

In a particular embodiment, this invention relates to compounds of Formula (Ib), wherein:

Z is N;

R¹ and R² are each independently methyl, ethyl, n-propyl, or n-butyl;

R³ and R⁴ are each hydrogen;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, and hydroxyl;

or pharmaceutically acceptable salts thereof.

In a particular embodiment, this invention relates to compounds of Formula (Ib), wherein:

Z is N;

R¹ and R² are each independently methyl, ethyl, n-propyl, or n-butyl;

R³ and R⁴ are each hydrogen;

or R³ and R⁴ taken together represent —CH₂CH₂—;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, hydroxyl, and —C(═N—CN)NH(C₁-C₄)alkyl;

or pharmaceutically acceptable salts thereof.

In another particular embodiment, this invention relates to compounds of Formula (Ib), wherein:

Z is CH or N;

R¹ and R² are each independently methyl, ethyl, n-propyl, or n-butyl;

R³ and R⁴ taken together represent —CH₂CH₂—;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, and hydroxyl;

or pharmaceutically acceptable salts thereof.

In another particular embodiment, this invention relates to compounds of Formula (Ib), wherein:

Z is CH;

R¹ and R² are each independently methyl, ethyl, n-propyl, or n-butyl;

R³ and R⁴ taken together represent —CH₂CH₂—;

R⁵ and R⁶ are each independently methyl, ethyl, n-propyl, or isopropyl; and

R⁷ is —N((C₁-C₄)alkyl)₂;

or pharmaceutically acceptable salts thereof.

In another embodiment, this invention also relates to compounds of Formula (Ib2):

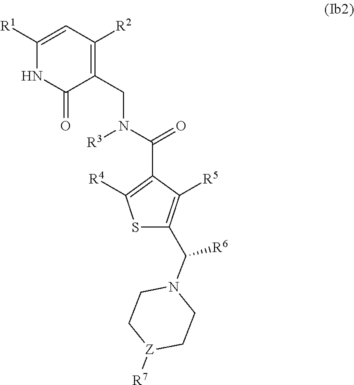

or pharmaceutically acceptable salts thereof, wherein Z, R¹, R², R³, R⁴, R⁵, R⁶, and R⁷ are defined according to Formula (I), provided that the compound is not N-((4,6-dimethyl-2-oxo-1,2-dihydropyridin-3-yl)methyl)-5-(1-(4-(dimethylamino)piperidin-1-yl)ethyl)-4-methylthiophene-3-carboxamide or N-((4,6-dimethyl-2-oxo-1,2-dihydropyridin-3-yl)methyl)-5-(1-(4-(dimethylamino)piperidin-1-yl)propyl)-4-methylthiophene-3-carboxamide, or stereoisomers or mixtures thereof of either of these compounds.

In a particular embodiment, this invention relates to compounds of Formula (Ib2), wherein:

Z is N;

R¹ and R² are each independently methyl, ethyl, n-propyl, or n-butyl;

R³ and R⁴ are each hydrogen;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, and hydroxyl;

or pharmaceutically acceptable salts thereof.

In a particular embodiment, this invention relates to compounds of Formula (Ib2), wherein:

Z is N;

R¹ and R² are each independently methyl, ethyl, n-propyl, or n-butyl;

R³ and R⁴ are each hydrogen;

or R³ and R⁴ taken together represent —CH₂CH₂—;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, hydroxyl, and —C(═N—CN)NH(C₁-C₄)alkyl;

or pharmaceutically acceptable salts thereof.

In another particular embodiment, this invention relates to compounds of Formula (Ib2), wherein:

Z is CH or N;

R¹ and R² are each independently methyl, ethyl, n-propyl, or n-butyl;

R³ and R⁴ taken together represent —CH₂CH₂—;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, and hydroxyl;

or pharmaceutically acceptable salts thereof.

In another particular embodiment, this invention relates to compounds of Formula (Ib2), wherein:

Z is CH;

R¹ and R² are each independently methyl, ethyl, n-propyl, or n-butyl;

R³ and R⁴ taken together represent —CH₂CH₂—;

R⁵ and R⁶ are each independently methyl, ethyl, n-propyl, or isopropyl; and

R⁷ is —N((C₁-C₄)alkyl)₂;

or pharmaceutically acceptable salts thereof.

In another embodiment, this invention also relates to compounds of Formula (Ic):

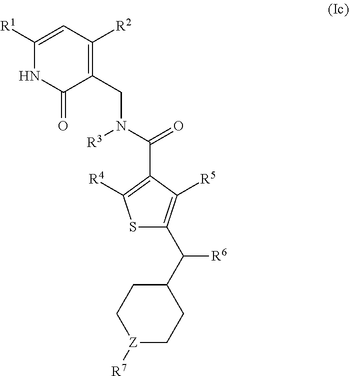

or pharmaceutically acceptable salts thereof, wherein Z, R¹, R², R³, R⁴, R⁵, R⁶, and R⁷ are defined according to Formula (I), provided that the compound is not N-((4,6-dimethyl-2-oxo-1,2-dihydropyridin-3-yl)methyl)-5-(1-(4-(dimethylamino)cyclohexyl)propyl)-4-methylthiophene-3-carboxamide, or stereoisomers or mixtures thereof.

In a particular embodiment, this invention relates to compounds of Formula (Ic), wherein:

Z is N;

R¹ and R² are each independently methyl, ethyl, n-propyl, or n-butyl;

R³ and R⁴ are each hydrogen;

or R³ and R⁴ taken together represent —CH₂CH₂—;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, hydroxyl, and —C(═N—CN)NH(C₁-C₄)alkyl;

or pharmaceutically acceptable salts thereof.

In another particular embodiment, this invention relates to compounds of Formula (Ic), wherein:

Z is N;

R¹ and R² are each independently methyl, ethyl, n-propyl, or n-butyl;

R³ and R⁴ are each hydrogen;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, and hydroxyl;

or pharmaceutically acceptable salts thereof.

In another embodiment, this invention also relates to compounds of Formula (Ic2):

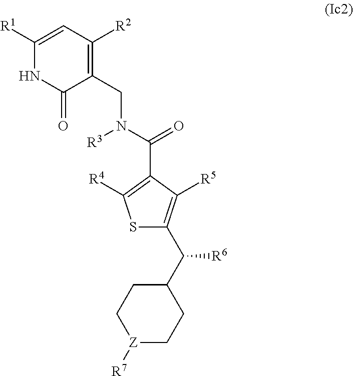

or pharmaceutically acceptable salts thereof, wherein Z, R¹, R², R³, R⁴, R⁵, R⁶, and R⁷ are defined according to Formula (I), provided that the compound is not N-((4,6-dimethyl-2-oxo-1,2-dihydropyridin-3-yl)methyl)-5-(1-(4-(dimethylamino)cyclohexyl)propyl)-4-methylthiophene-3-carboxamide, or stereoisomers or mixtures thereof.

In a particular embodiment, this invention relates to compounds of Formula (Ic2), wherein:

Z is N;

R¹ and R² are each independently methyl, ethyl, n-propyl, or n-butyl;

R³ and R⁴ are each hydrogen;

or R³ and R⁴ taken together represent —CH₂CH₂—;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, hydroxyl, and —C(═N—CN)NH(C₁-C₄)alkyl;

or pharmaceutically acceptable salts thereof.

In another particular embodiment, this invention relates to compounds of Formula (Ic2), wherein:

Z is N;

R¹ and R² are each independently methyl, ethyl, n-propyl, or n-butyl;

R³ and R⁴ are each hydrogen;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, and hydroxyl;

or pharmaceutically acceptable salts thereof.

In another embodiment, this invention also relates to compounds of Formula (Id):

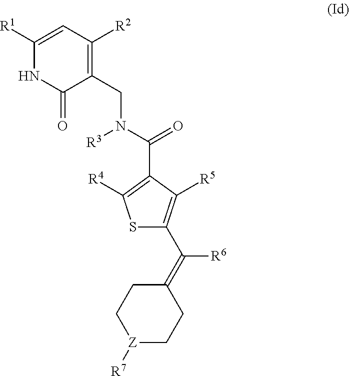

or pharmaceutically acceptable salts thereof, wherein Z, R¹, R², R³, R⁴, R⁵, R⁶, and R⁷ are defined according to Formula (I).

In a particular embodiment, this invention relates to compounds of Formula (Id), wherein:

Z is N;

R¹ and R² are each independently methyl, ethyl, n-propyl, or n-butyl;

R³ and R⁴ are each hydrogen;

or R³ and R⁴ taken together represent —CH₂CH₂—;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, hydroxyl, and —C(═N—CN)NH(C₁-C₄)alkyl;

or pharmaceutically acceptable salts thereof.

In another particular embodiment, this invention relates to compounds of Formula (Id), wherein:

Z is CH or N;

R¹ and R² are each independently methyl, ethyl, n-propyl, or n-butyl;

R³ and R⁴ are each hydrogen;

R⁵ and R⁶ are each independently methyl, ethyl, n-propyl, or isopropyl; and

R⁷ is selected from the group consisting of halo(C₁-C₄)alkyl, —N((C₁-C₄)alkyl)₂, and hydroxyl;

or pharmaceutically acceptable salts thereof.

Specific compounds of this invention include:
5-(1-(1-(2,2-difluoropropyl)piperidin-4-ylidene)propyl)-N-((4,6-dimethyl-2-oxo-1,2-dihydropyridin-3-yl)methyl)-4-methylthiophene-3-carboxamide;
N-((4,6-dimethyl-2-oxo-1,2-dihydropyridin-3-yl)methyl)-5-(1-(1-(2-fluoro-2-methylpropyl)piperidin-4-ylidene)propyl)-4-methylthiophene-3-carboxamide;
N-((4,6-dimethyl-2-oxo-1,2-dihydropyridin-3-yl)methyl)-4-methyl-5-(1-(1-(2,2,2-trifluoroethyl)piperidin-4-ylidene)propyl)thiophene-3-carboxamide;
(R)-5-(1-(1-(2,2-difluoropropyl)piperidin-4-yl)propyl)-N-((4,6-dimethyl-2-oxo-1,2-dihydropyridin-3-yl)methyl)-4-methylthiophene-3-carboxamide;
(S)-5-(1-(1-(2,2-difluoropropyl)piperidin-4-yl)propyl)-N-((4,6-dimethyl-2-oxo-1,2-dihydropyridin-3-yl)methyl)-4-methylthiophene-3-carboxamide;
N-((4,6-dimethyl-2-oxo-1,2-dihydropyridin-3-yl)methyl)-5-(1-(1-(2-fluoro-2-methylpropyl)piperidin-4-yl)propyl)-4-methylthiophene-3-carboxamide;
N-((4,6-dimethyl-2-oxo-1,2-dihydropyridin-3-yl)methyl)-5-(1-(1-(dimethylamino)piperidin-4-ylidene)propyl)-4-methylthiophene-3-carboxamide;
N-((4,6-dimethyl-2-oxo-1,2-dihydropyridin-3-yl)methyl)-5-(1-(1-hydroxypiperidin-4-ylidene)propyl)-4-methylthiophene-3-carboxamide;
5-((4,6-dimethyl-2-oxo-1,2-dihydropyridin-3-yl)methyl)-2-(1-(4-(dimethylamino)piperidin-1-yl)propyl)-3-methyl-6,7-dihydrothieno[3,2-c]pyridin-4 (5H)-one;
N-((4,6-dimethyl-2-oxo-1,2-dihydropyridin-3-yl)methyl)-5-(1-(4-hydroxycyclohexylidene)propyl)-4-methylthiophene-3-carboxamide;
N-((4,6-dimethyl-2-oxo-1,2-dihydropyridin-3-yl)methyl)-4-methyl-5-(1-(1-(2,2,2-trifluoroethyl)piperidin-4-yl)propyl)thiophene-3-carboxamide;
N-((4,6-dimethyl-2-oxo-1,2-dihydropyridin-3-yl)methyl)-4-methyl-5-(1-(1-(oxazol-2-ylmethyl)piperidin-4-ylidene)propyl)thiophene-3-carboxamide;
N-((4,6-dimethyl-2-oxo-1,2-dihydropyridin-3-yl)methyl)-4-methyl-5-(1-(1-(pyrimidin-2-yl)piperidin-4-ylidene)propyl)thiophene-3-carboxamide;
5-(1-(1-(2,2-difluoroethyl)piperidin-4-yl)propyl)-N-((4,6-dimethyl-2-oxo-1,2-dihydropyridin-3-yl)methyl)-4-methylthiophene-3-carboxamide;
5-(1-(1-(N′-cyano-N-methylcarbamimidoyl)piperidin-4-ylidene)propyl)-N-((4,6-dimethyl-2-oxo-1,2-dihydropyridin-3-yl)methyl)-4-methylthiophene-3-carboxamide;
2-(1-(1-(2,2-difluoropropyl)piperidin-4-yl)propyl)-5-(4,6-dimethyl-2-oxo-1,2-dihydropyridin-3-yl)methyl)-3-methyl-6,7-dihydrothieno[3,2-c]pyridin-4(5H)-one;
5-(4,6-dimethyl-2-oxo-1,2-dihydropyridin-3-yl)methyl)-2-(1-(1-(2-fluoropropyl)piperidin-4-yl)propyl)-3-methyl-6,7-dihydrothieno[3,2-c]pyridin-4(5H)-one;
N′-cyano-4-(1-(5-(4,6-dimethyl-2-oxo-1,2-dihydropyridin-3-yl)methyl)-3-methyl-4-oxo-4,5,6,7-tetrahydrothieno[3,2-c]pyridin-2-yl)propyl)-N-methylpiperidine-1-carboximidamide;
5-(1-(1-(N′-cyano-N-methylcarbamimidoyl)piperidin-4-yl)propyl)-N-((4,6-dimethyl-2-oxo-1,2-dihydropyridin-3-yl)methyl)-4-methylthiophene-3-carboxamide;
5-(1-(1-(2,2-difluoroethyl)piperidin-4-ylidene)propyl)-N-((4,6-dimethyl-2-oxo-1,2-dihydropyridin-3-yl)methyl)-4-methylthiophene-3-carboxamide;
(R)-5-(1-(1-(2,2-difluoroethyl)piperidin-4-yl)propyl)-N-((4,6-dimethyl-2-oxo-1,2-dihydropyridin-3-yl)methyl)-4-methylthiophene-3-carboxamide;
(R)-5-(1-(1-(2,2-difluoroethyl)piperidin-4-yl)ethyl)-N-((4,6-dimethyl-2-oxo-1,2-dihydropyridin-3-yl)methyl)-4-methylthiophene-3-carboxamide; and
(R)-5-(1-(1-(2,2-difluoropropyl)piperidin-4-yl)ethyl)-N-((4,6-dimethyl-2-oxo-1,2-dihydropyridin-3-yl)methyl)-4-methylthiophene-3-carboxamide;

or pharmaceutically acceptable salts thereof.

Typically, but not absolutely, the salts of the present invention are pharmaceutically acceptable salts. Salts of the disclosed compounds containing a basic amine or other basic functional group may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycolates, tartrates mandelates, and sulfonates, such as xylenesulfonates, methanesulfonates, propanesulfonates, naphthalene-1-sulfonates and naphthalene-2-sulfonates.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

The compound of Formula (I) or a salt thereof may exist in stereoisomeric forms (e.g., it contains one or more asymmetric carbon atoms). The individual stereoisomers (enantiomers and diastereomers) and mixtures of these are included within the scope of the present invention. Likewise, it is understood that a compound or salt of Formula (I) may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. It is to be understood that the present invention includes all combinations and subsets of the particular groups defined hereinabove.

The subject invention also includes isotopically-labeled compounds, which are identical to those recited in Formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulfur, fluorine, chlorine, and iodine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I, and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of Formula (I) and following of this invention can generally be prepared by carrying out the procedures disclosed in the Schemes and/or in the Examples below, by substituting a readily available isotopically labeled reagent for a non-isotopically labeled reagent.

The invention further provides a pharmaceutical composition (also referred to as pharmaceutical formulation) comprising a compound of Formula (I) or pharmaceutically acceptable salt thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

Pharmaceutical compositions may be in unit dose form containing a predetermined amount of active ingredient per unit dose. Such a unit may contain a therapeutically effective dose of the compound of Formula (I) or salt thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound or salt thereof of the invention or the pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound or salt thereof of the invention in a suitably flavoured aqueous solution, while elixirs are prepared through the use of a non-toxic alcoholic vehicle. Suspensions can be formulated by dispersing the compound or salt of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

In accordance with another aspect of the invention there is provided a process for the preparation of a pharmaceutical composition comprising mixing (or admixing) a compound of Formula (I) or salt thereof with at least one excipient.

The present invention also provides a method of treatment in a mammal, especially a human. The compounds and compositions of the invention are used to treat cellular proliferation diseases. Disease states which can be treated by the methods and compositions provided herein include, but are not limited to, cancer (further discussed below), autoimmune disease, fungal disorders, arthritis, graft rejection, inflammatory bowel disease, proliferation induced after medical procedures, including, but not limited to, surgery, angioplasty, and the like. It is appreciated that in some cases the cells may not be in a hyper or hypo proliferation state (abnormal state) and still requires treatment. For example, during wound healing, the cells may be proliferating “normally”, but proliferation enhancement may be desired. Thus, in one embodiment, the invention herein includes application to cells or individuals afflicted or impending affliction with any one of these disorders or states.

The compositions and methods provided herein are particularly deemed useful for the treatment of cancer including tumors such as prostate, breast, brain, skin, cervical carcinomas, testicular carcinomas, etc. They are particularly useful in treating metastatic or malignant tumors. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one or related of the above identified conditions.

The instant compounds can be combined with or co-administered with other therapeutic agents, particularly agents that may enhance the activity or time of disposition of the compounds. Combination therapies according to the invention comprise the administration of at least one compound of the invention and the use of at least one other treatment method. In one embodiment, combination therapies according to the invention comprise the administration of at least one compound of the invention and surgical therapy. In one embodiment, combination therapies according to the invention comprise the administration of at least one compound of the invention and radiotherapy. In one embodiment, combination therapies according to the invention comprise the administration of at least one compound of the invention and at least one supportive care agent (e.g., at least one anti-emetic agent). In one embodiment, combination therapies according to the present invention comprise the administration of at least one compound of the invention and at least one other chemotherapeutic agent. In one particular embodiment, the invention comprises the administration of at least one compound of the invention and at least one anti-neoplastic agent. In yet another embodiment, the invention comprises a therapeutic regimen where the EZH2 inhibitors of this disclosure are not in and of themselves active or significantly active, but when combined with another therapy, which may or may not be active as a standalone therapy, the combination provides a useful therapeutic outcome.

By the term “co-administering” and derivatives thereof as used herein refers to either simultaneous administration or any manner of separate sequential administration of an EZH2 inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of specified cancers in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracycline, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; DNA methyltransferase inhibitors such as azacitidine and decitabine; signal transduction pathway inhibitors; non-receptor tyrosine kinase angiogenesis inhibitors; immunotherapeutic agents; proapoptotic agents; and cell cycle signaling inhibitors.

Typically, any chemotherapeutic agent that has activity against a susceptible neoplasm being treated may be utilized in combination with the compounds the invention, provided that the particular agent is clinically compatible with therapy employing a compound of the invention. Typical anti-neoplastic agents useful in the present invention include, but are not limited to: alkylating agents, anti-metabolites, antitumor antibiotics, antimitotic agents, nucleoside analogues, topoisomerase I and II inhibitors, hormones and hormonal analogues; retinoids, histone deacetylase inhibitors; signal transduction pathway inhibitors including inhibitors of cell growth or growth factor function, angiogenesis inhibitors, and serine/threonine or other kinase inhibitors; cyclin dependent kinase inhibitors; antisense therapies and immunotherapeutic agents, including monoclonals, vaccines or other biological agents.

Nucleoside analogues are those compounds which are converted to deoxynucleotide triphosphates and incorporated into replicating DNA in place of cytosine. DNA methyltransferases become covalently bound to the modified bases resulting in an inactive enzyme and reduced DNA methylation. Examples of nucleoside analogues include azacitidine and decitabine which are used for the treatment of myelodysplastic disorder. Histone deacetylase (HDAC) inhibitors include vorinostat, for the treatment of cutaneous T-cell lymphoma. HDACs modify chromatin through the deacetylation of histones. In addition, they have a variety of substrates including numerous transcription factors and signaling molecules. Other HDAC inhibitors are in development.

Signal transduction pathway inhibitors are those inhibitors which block or inhibit a chemical process which evokes an intracellular change. As used herein this change is cell proliferation or differentiation or survival. Signal transduction pathway inhibitors useful in the present invention include, but are not limited to, inhibitors of receptor tyrosine kinases, non-receptor tyrosine kinases, SH2/SH3 domain blockers, serine/threonine kinases, phosphatidyl inositol-3-OH kinases, myoinositol signaling, and Ras oncogenes. Signal transduction pathway inhibitors may be employed in combination with the compounds of the invention in the compositions and methods described above.

Receptor kinase angiogenesis inhibitors may also find use in the present invention. Inhibitors of angiogenesis related to VEGFR and TIE-2 are discussed above in regard to signal transduction inhibitors (both are receptor tyrosine kinases). Other inhibitors may be used in combination with the compounds of the invention. For example, anti-VEGF antibodies, which do not recognize VEGFR (the receptor tyrosine kinase), but bind to the ligand; small molecule inhibitors of integrin (alphaᵥbeta₃) that inhibit angiogenesis; endostatin and angiostatin (non-RTK) may also prove useful in combination with the compounds of the invention. One example of a VEGFR antibody is bevacizumab) (AVASTIN®).

Several inhibitors of growth factor receptors are under development and include ligand antagonists, antibodies, tyrosine kinase inhibitors, anti-sense oligonucleotides and aptamers. Any of these growth factor receptor inhibitors may be employed in combination with the compounds of the invention in any of the compositions and methods/uses described herein. Trastuzumab (Herceptin®) is an example of an anti-erbB2 antibody inhibitor of growth factor function. One example of an anti-erbB1 antibody inhibitor of growth factor function is cetuximab (Erbitux™, C225). Bevacizumab (Avastin®) is an example of a monoclonal antibody directed against VEGFR. Examples of small molecule inhibitors of epidermal growth factor receptors include but are not limited to lapatinib (Tykerb®) and erlotinib (TARCEVA®). Imatinib mesylate (GLEEVEC®) is one example of a PDGFR inhibitor. Examples of VEGFR inhibitors include pazopanib (Votrient®), ZD6474, AZD2171, PTK787, sunitinib and sorafenib.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325 (1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Int. Med., 111:273, 1989) and for the treatment of breast cancer (Holmes et al., J. Nat. Cancer Inst., 83:1797, 1991.). It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leukopenialeukopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leukopenialeukopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leukopenialeukopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leukopenialeukopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leukopenialeukopenia, thrombocytopenia, and anemia, is the most common dose limiting side effect of gemcitabine administration.

Methotrexate, N-[4[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leukopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Provided herein are methods of treatment or prevention of autoimmune and inflammatory conditions and diseases that can be improved by inhibiting EZH1 and/or EZH2 and thereby, e.g., modulate the level of expression of methylation activated and methylation repressed target genes, or modulate the activity of signalling proteins. A method may comprise administering to a human, e.g. a human in need thereof, a therapeutically effective amount of an agent described herein.

Inflammation represents a group of vascular, cellular and neurological responses to trauma. Inflammation can be characterised as the movement of inflammatory cells such as monocytes, neutrophils and granulocytes into the tissues. This is usually associated with reduced endothelial barrier function and oedema into the tissues. Inflammation can be classified as either acute or chronic. Acute inflammation is the initial response of the body to harmful stimuli and is achieved by the increased movement of plasma and leukocytes from the blood into the injured tissues. A cascade of biochemical event propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterised by simultaneous destruction and healing of the tissue from the inflammatory process.

When occurring as part of an immune response to infection or as an acute response to trauma, inflammation can be beneficial and is normally self-limiting. However, inflammation can be detrimental under various conditions. This includes the production of excessive inflammation in response to infectious agents, which can lead to significant organ damage and death (for example, in the setting of sepsis). Moreover, chronic inflammation is generally deleterious and is at the root of numerous chronic diseases, causing severe and irreversible damage to tissues. In such settings, the immune response is often directed against self-tissues (autoimmunity), although chronic responses to foreign entities can also lead to bystander damage to self tissues.

The aim of anti-inflammatory therapy is therefore to reduce this inflammation, to inhibit autoimmunity when present and to allow for the physiological process or healing and tissue repair to progress.

The agents may be used to treat inflammation of any tissue and organs of the body, including musculoskeletal inflammation, vascular inflammation, neural inflammation, digestive system inflammation, ocular inflammation, inflammation of the reproductive system, and other inflammation, as exemplified below.

Musculoskeletal inflammation refers to any inflammatory condition of the musculoskeletal system, particularly those conditions affecting skeletal joints, including joints of the hand, wrist, elbow, shoulder, jaw, spine, neck, hip, knew, ankle, and foot, and conditions affecting tissues connecting muscles to bones such as tendons. Examples of musculoskeletal inflammation which may be treated with compounds of the invention include arthritis (including, for example, osteoarthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular inflammation refers to inflammation of any structure of the eye, including the eye lids. Examples of ocular inflammation which may be treated in this invention include blepharitis, blepharochalasis, conjunctivitis, dacryoadenitis, keratitis, keratoconjunctivitis sicca (dry eye), scleritis, trichiasis, and uveitis.

Examples of inflammation of the nervous system which may be treated in this invention include encephalitis, Guillain-Barre syndrome, meningitis, neuromyotonia, narcolepsy, multiple sclerosis, myelitis and schizophrenia.

Examples of inflammation of the vasculature or lymphatic system which may be treated in this invention include arthrosclerosis, arthritis, phlebitis, vasculitis, and lymphangitis.

Examples of inflammatory conditions of the digestive system which may be treated in this invention include cholangitis, cholecystitis, enteritis, enterocolitis, gastritis, gastroenteritis, ileitis, and proctitis.

Examples of inflammatory conditions of the reproductive system which may be treated in this invention include cervicitis, chorioamnionitis, endometritis, epididymitis, omphalitis, oophoritis, orchitis, salpingitis, tubo-ovarian abscess, urethritis, vaginitis, vulvitis, and vulvodynia.

The agents may be used to treat autoimmune conditions having an inflammatory component. Such conditions include acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, goodpasture's syndrome, Grave's disease, Guillain-Barre syndrome, Hashimoto's disease, Henoch-Schönlein purpura, Kawasaki's disease, lupus erythematosus, microscopic colitis, microscopic polyarteritis, mixed connective tissue disease, multiple sclerosis, myasthenia gravis, opsocionus myoclonus syndrome, optic neuritis, ord's thyroiditis, pemphigus, polyarteritis nodosa, polymyalgia, Reiter's syndrome, Sjogren's syndrome, temporal arteritis, Wegener's granulomatosis, warm autoimmune haemolytic anemia, interstitial cystitis, lyme disease, morphea, sarcoidosis, scleroderma, ulcerative colitis, and vitiligo.

The agents may be used to treat T-cell mediated hypersensitivity diseases having an inflammatory component. Such conditions include contact hypersensitivity, contact dermatitis (including that due to poison ivy), uticaria, skin allergies, respiratory allergies (hayfever, allergic rhinitis) and gluten-sensitive enteropathy (Celliac disease).

Other inflammatory conditions which may be treated in this invention include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasia, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, astopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis.

Preferred treatments include any one of treatment of transplant rejection, psoriatic arthritis, multiple sclerosis, Type 1 diabetes, asthma, systemic lupus erythematosis, chronic pulmonary disease, and inflammation accompanying infectious conditions (e.g., sepsis).

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the Formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of Formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavoring agents.

A therapeutically effective amount of a compound of the present invention will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of Formula (I) for the treatment of anemia will generally be in the range of 0.001 to 100 mg/kg body weight of recipient per day, suitably in the range of 0.01 to 10 mg/kg body weight per day. For a 70 kg adult mammal, the actual amount per day would suitably be from 7 to 700 mg and this amount may be given in a single dose per day or in a number (such as two, three, four, five or six) of sub-doses per day such that the total daily dose is the same. An effective amount of a salt or solvate, etc., may be determined as a proportion of the effective amount of the compound of Formula (I)per se. It is envisaged that similar dosages would be appropriate for treatment of the other conditions referred to above.

### Definitions

Terms are used within their accepted meanings. The following definitions are meant to clarify, but not limit, the terms defined.

As used herein, the term “alkyl” represents a saturated, straight or branched hydrocarbon moiety having the specified number of carbon atoms. The term “(C₁-C₆)alkyl” refers to an alkyl moiety containing from 1 to 6 carbon atoms. Exemplary alkyls include, but are not limited to methyl, ethyl, n-propyl, isopropyl, n-butyl, isobutyl, s-butyl, t-butyl, pentyl, and hexyl.

When the term “alkyl” is used in combination with other substituent groups, such as “halo(C₁-C₄)alkyl”, the term “alkyl” is intended to encompass a divalent straight or branched-chain hydrocarbon radical, wherein the point of attachment is through the alkyl moiety. The term “halo(C₁-C₄)alkyl” is intended to mean a radical having one or more halogen atoms, which may be the same or different, at one or more carbon atoms of an alkyl moiety containing from 1 to 4 carbon atoms, which is a straight or branched-chain carbon radical. Examples of “halo(C₁-C₄)alkyl” groups useful in the present invention include, but are not limited to, —CF₃ (trifluoromethyl), —CCl₃ (trichloromethyl), 1,1-difluoroethyl, 2-fluoro-2-methylpropyl, 2,2-difluoropropyl, 2,2,2-trifluoroethyl, and hexafluoroisopropyl.

The terms “halogen” and “halo” represent chloro, fluoro, bromo, or iodo substituents. “Hydroxy” or “hydroxyl” is intended to mean the radical —OH.

As used herein, the term “optionally” means that the subsequently described event(s) may or may not occur, and includes both event(s) that occur and event(s) that do not occur.

As used herein, the term “treatment” refers to alleviating the specified condition, eliminating or reducing one or more symptoms of the condition, slowing or eliminating the progression of the condition, and delaying the reoccurrence of the condition in a previously afflicted or diagnosed patient or subject.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. Additionally, the active ingredient may be presented as a pharmaceutical composition.

### Compound Preparation

### Abbreviations

AcOH acetic acid
Boc tert-butyloxycarbonyl
Boc₂O di-tert-butyl dicarbonate
CH₃CN acetonitrile
CH₃NO₂ nitromethane
Cs₂CO₃ cesium carbonate
DCM dichloromethane
DIBAL-H diisobutylaluminium hydride
DMAP 4-dimethylaminopyridine
DMF N,N-dimethylformamide
DMSO dimethyl sulfoxide
EDC N-(3-dimethylaminopropyl)-N′-ethylcarbodiimide hydrochloride
ES electrospray
Et₃N triethylamine
EtOAc ethyl acetate
EtOCOCl ethyl chloroformate
EtOH ethanol
h hour(s)
H₂ hydrogen gas
HCl hydrochloric acid
H₂O water
H₂SO₄ sulfuric acid
HOAt 1-hydroxy-7-azabenzotriazole
HPLC high-performance liquid chromatography
In(OTf)₃ indium (III) trifluoromethanesulfonate
K₂CO₃ potassium carbonate
KOAc potassium acetate
KOtBu potassium tert-butoxide
LCMS liquid chromatography mass spectrometry
LiBH₄ lithium borohydride
LiClO₄ lithium perchlorate
MeOH methanol
MgSO₄ magnesium sulfate
min minute(s)
MS mass spectrometry
NaBH₄ sodium borohydride
NaBH₃CN sodium cyanoborohydride
NaBH(OAc)₃ sodium triacetoxyborohydride
Na₂CO₃ sodium carbonate
NaHCO₃ sodium bicarbonate
NaHMDS sodium bis(trimethylsilyl)amide
Na₂HPO₄ disodium phosphate
NaNO₂ sodium nitrite
NaOH sodium hydroxide
Na₂SO₄ sodium sulfate
NBS N-bromosuccinimide
NH₄Cl ammonium chloride
NH₄OAc ammonium acetate
NH₄OH ammonium hydroxide
NMM N-methylmorpholine
2-NTf₂-pyridine 1,1,1-trifluoro-N-(pyridin-2-yl)-N-((trifluoromethyl)sulfonyl)methanesulfonamide
Pd/C palladium on carbon
PdCl₂(dppf) [1,1′-bis(diphenylphosphino)ferrocene]dichloropalladium(II)
Pd(PPh₃)₄ tetrakis(triphenylphosphine)palladium(0)
PhH benzene
P₂O₅ phosphorus pentoxide
POCl₃ phosphoryl chloride
pyr pyridine
RT room temperature
SOCl₂ thionyl chloride
TFA trifluoroacetic acid
Tf₂O trifluoromethanesulfonic anhydride
THF tetrahydrofuran
TiCl₄ titanium(IV) chloride
TMSCl trimethylsilyl chloride
Ti(OiPr)₄ titanium(IV) isopropoxide

### Generic Synthesis Schemes

The compounds of this invention may be made by a variety of methods, including well-known standard synthetic methods. Illustrative general synthetic methods are set out below and then specific compounds of the invention are prepared in the working examples. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts, (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention. Starting materials are commercially available or are made from commercially available starting materials using methods known to those skilled in the art.

The compounds of Formula (Id) can be prepared according to Scheme 1 or analogous methods. Esterification of an appropriately substituted thiophene-3-carboxylic acid provides the corresponding ester. An indium-mediated acylation reaction with an appropriately substituted anyhydride (or acylchloride) affords the 5-acylthiophene. A McMurray coupling with an appropriately substituted ketone affords the tetra-substituted olefin. Alkylation with appropriately substituted triflates (or alkyl halides) or reductive amination with appropriately substituted aldehydes furnishes the substituted derivatives. Saponification of the ester, followed by coupling of the resultant carboxylic acid with an appropriately substituted amine affords compounds of Formula (Id).

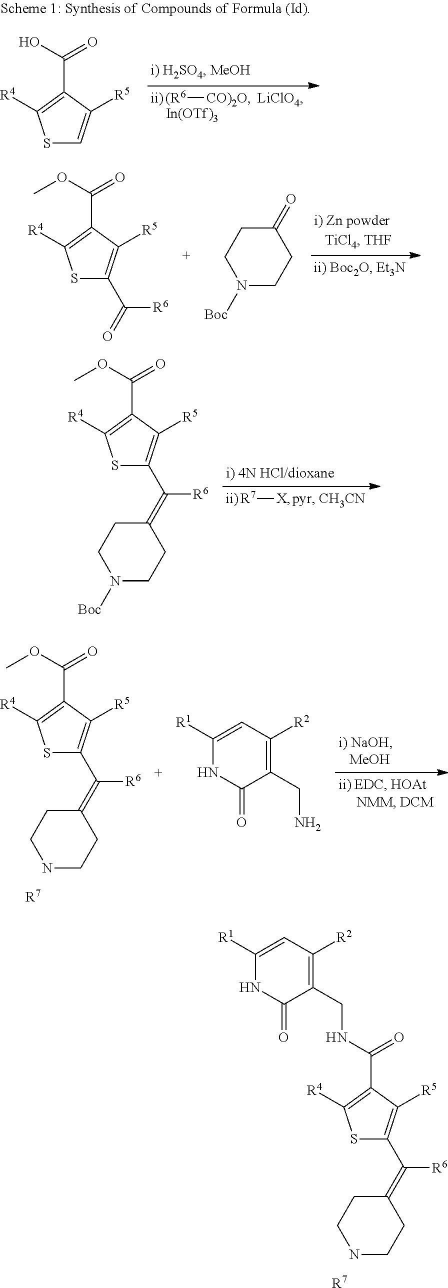

The compounds of Formula (Ic) can be prepared according to Scheme 2 or analogous methods. Formation of an appropriately substituted ketone from its corresponding Weinreb amide is accomplished with an appropriate Grignard (or alkyllithium) reagent. Formation of the corresponding vinyl triflate, followed by palladium-mediated coupling to an appropriately substituted bromothiophene affords the tri-substituted olefin. Reduction of the olefin, followed by alkylation with appropriately substituted triflates (or alkyl halides) or reductive amination with appropriately substituted aldehydes furnishes the substituted derivatives. Saponification of the ester, followed by coupling of the resultant carboxylic acid with an appropriately substituted amine affords compounds of Formula (Ic).

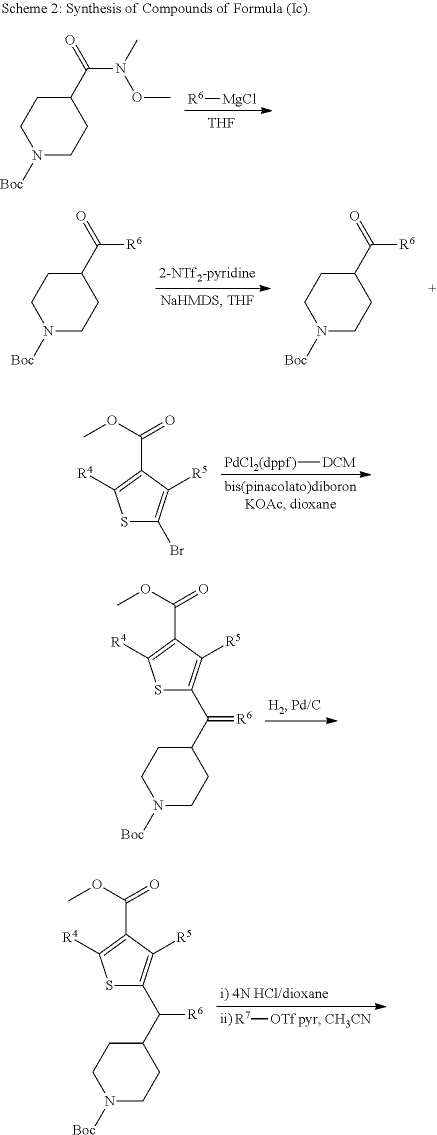

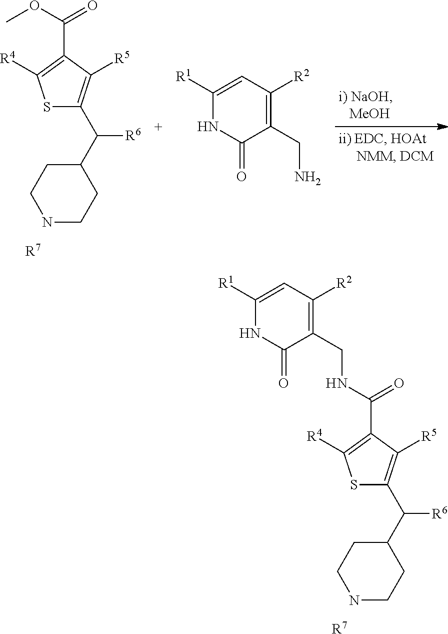

The compounds of Formula (I) wherein R³ and R⁴ taken together represent —CH₂CH₂— can be prepared according to Scheme 3 or analogous methods. Condensation of an appropriately substituted thiophenecarbaldehyde with nitromethane provides the corresponding nitrovinyl thiophene. Reduction of the nitrovinyl, followed by trapping of the resultant amine yields the corresponding urethane. Treatment of the urethane with POCl₃/P₂O₅ furnishes the lactam. An indium-mediated acylation reaction with an appropriately substituted anyhydride (or acylchloride) affords the 5-acylthiophene. Reductive amination with an appropriately substituted amine or a McMurray coupling with an appropriately substituted ketone affords the elaborated thiophenelactams. Alkylation of the lactam nitrogen with an appropriately substituted alkylhalide, followed by removal of the benzyl protecting group affords compounds of Formula (I).

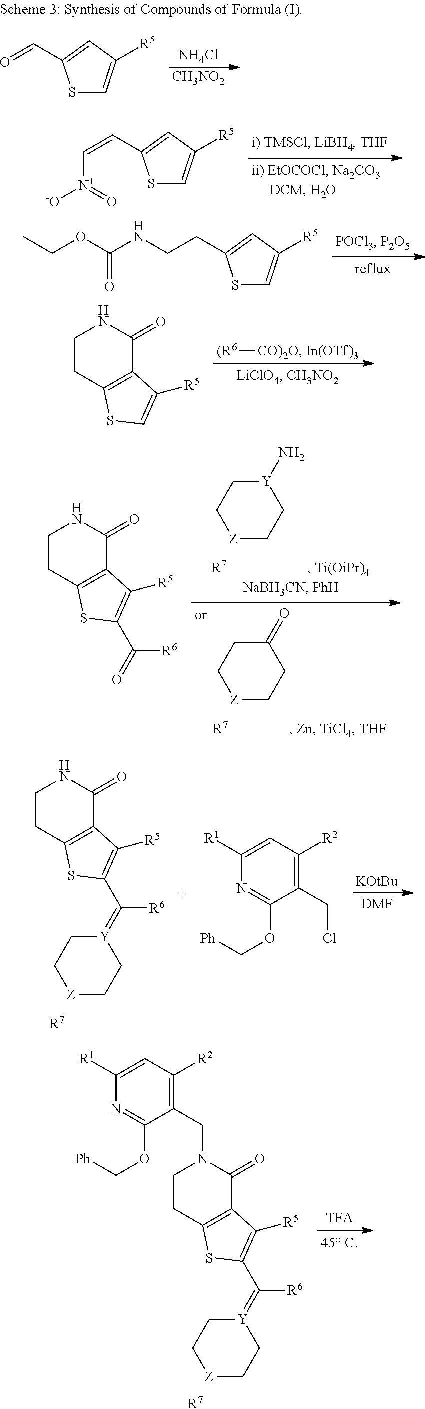

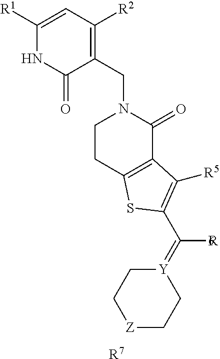

Compounds of Formula (Ic2) can be prepared according to Scheme 4 or analogous methods. An iridium-mediated borylation, followed by a Suzuki coupling with an appropriately substituted triflate gives the corresponding coupled olefin. An iridium-mediated asymmetric reduction of the olefin, followed by removal of the Boc-protecting group provides the piperidine. Alkylation with appropriately substituted triflates (or alkyl halides) or reductive amination with appropriately substituted aldehydes furnishes the substituted derivatives. Saponification of the ester, followed by coupling of the resultant carboxylic acid with an appropriately substituted amine affords compounds of Formula (Ic2).

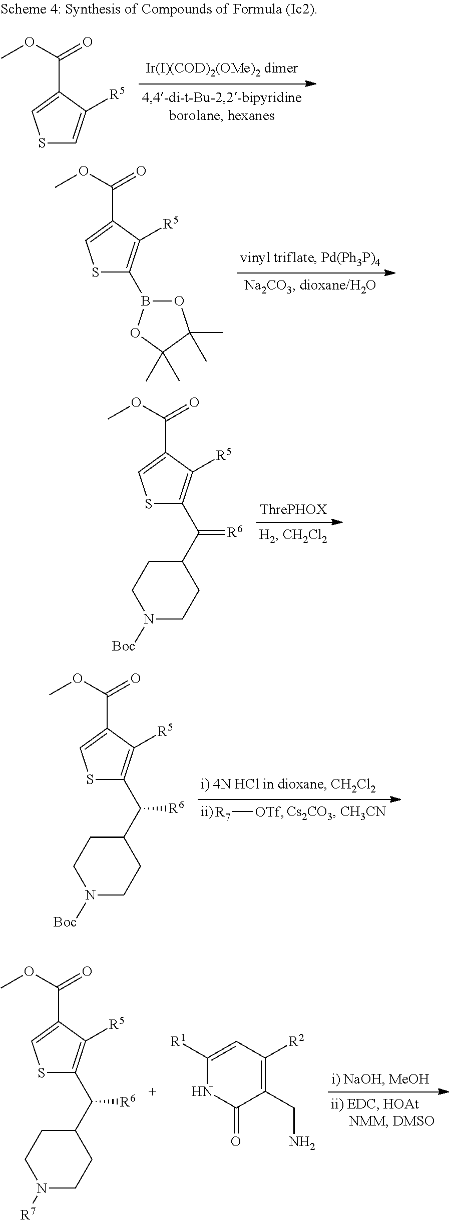

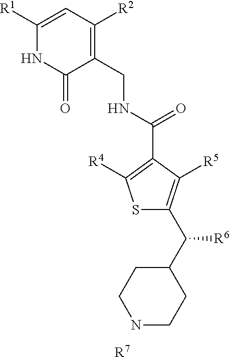

### EXPERIMENTALS

The following guidelines apply to all experimental procedures described herein. All reactions were conducted under a positive pressure of nitrogen using oven-dried glassware, unless otherwise indicated. Temperatures designated are external (i.e. bath temperatures), and are approximate. Air and moisture-sensitive liquids were transferred via syringe. Reagents were used as received. Solvents utilized were those listed as “anhydrous” by vendors. Molarities listed for reagents in solutions are approximate, and were used without prior titration against a corresponding standard. All reactions were agitated by stir bar, unless otherwise indicated. Heating was conducted using heating baths containing silicon oil, unless otherwise indicated. Reactions conducted by microwave irradiation (0-400 W at 2.45 GHz) were done so using a Biotage Initiator™ 2.0 instrument with Biotage microwave EXP vials (0.2-20 mL) and septa and caps. Irradiation levels utilized (i.e. high, normal, low) based on solvent and ionic charge were based on vendor specifications. Cooling to temperatures below −70° C. was conducted using dry ice/acetone or dry ice/2-propanol. Magnesium sulfate and sodium sulfate used as drying agents were of anhydrous grade, and were used interchangeably. Solvents described as being removed “in vacuo” or “under reduced pressure” were done so by rotary evaporation.

Preparative normal phase silica gel chromatography was carried out using either a Teledyne ISCO CombiFlash Companion instrument with RediSep or ISCO Gold silica gel cartridges (4 g-330 g), or an Analogix IF280 instrument with SF25 silica gel cartridges (4 g-3-00 g), or a Biotage SP1 instrument with HP silica gel cartridges (10 g-100 g). Purification by reverse phase HPLC was conducted using a YMC-pack column (ODS-A 75×30 mm) as solid phase, unless otherwise noted. A mobile phase of 25 mL/min A (CH₃CN-0.1% TFA): B (water-0.1% TFA), 10-80% gradient A (10 min) was utilized with UV detection at 214 nM, unless otherwise noted.

A PE Sciex API 150 single quadrupole mass spectrometer (PE Sciex, Thornhill, Ontario, Canada) was operated using electrospray ionization in the positive ion detection mode. The nebulizing gas was generated from a zero air generator (Balston Inc., Haverhill, Mass., USA) and delivered at 65 psi and the curtain gas was high purity nitrogen delivered from a Dewar liquid nitrogen vessel at 50 psi. The voltage applied to the electrospray needle was 4.8 kV. The orifice was set at 25 V and mass spectrometer was scanned at a rate of 0.5 scan/sec using a step mass of 0.2 amu and collecting profile data.

Method A LCMS. Samples were introduced into the mass spectrometer using a CTC PAL autosampler (LEAP Technologies, Carrboro, N.C.) equipped with a Hamilton 10 uL syringe which performed the injection into a Valco 10-port injection valve. The HPLC pump was a Shimadzu LC-10ADvp (Shimadzu Scientific Instruments, Columbia, Md.) operated at 0.3 mL/min and a linear gradient 4.5% A to 90% B in 3.2 min. with a 0.4 min. hold. The mobile phase was composed of 100% (H₂O 0.02% TFA) in vessel A and 100% (CH₃CN 0.018% TFA) in vessel B. The stationary phase is Aquasil (C18) and the column dimensions were 1 mm×40 mm. Detection was by UV at 214 nm, evaporative light-scattering (ELSD) and MS.

Method B, LCMS. Alternatively, an Agilent 1100 analytical HPLC system with an LC/MS was used and operated at 1 mL/min and a linear gradient 5% A to 100% B in 2.2 min with a 0.4 min hold. The mobile phase was composed of 100% (H₂O 0.02% TFA) in vessel A and 100% (CH₃CN 0.018% TFA) in vessel B. The stationary phase was Zobax (C8) with a 3.5 um partical size and the column dimensions were 2.1 mm×50 mm. Detection was by UV at 214 nm, evaporative light-scattering (ELSD) and MS.

Method C, LCMS. Alternatively, an MDSSCIEX API 2000 equipped with a capillary column of (50×4.6 mm, 5 μm) was used. HPLC was done on Agilent-1200 series UPLC system equipped with column Zorbax SB-C18 (50×4.6 mm, 1.8 μm) eluting with CH₃CN: NH₄OAc buffer. The reactions were performed in the microwave (CEM, Discover).

¹H-NMR spectra were recorded at 400 MHz using a Bruker AVANCE 400 MHz instrument, with ACD Spect manager v. 10 used for reprocessing. Multiplicities indicated are: s=singlet, d=doublet, t=triplet, q=quartet, quint=quintet, sxt=sextet, m=multiplet, dd=doublet of doublets, dt=doublet of triplets etc. and br indicates a broad signal. All NMRs in DMSO-d₆ unless otherwise noted.

Analytical HPLC: Products were analyzed by Agilent 1100 Analytical Chromatography system, with 4.5×75 mm Zorbax XDB-C18 column (3.5 um) at 2 mL/min with a 4 min gradient from 5% CH₃CN (0.1% formic acid) to 95% CH₃CN (0.1% formic acid) in H₂O (0.1% formic acid) and a 1 min hold.

### Intermediates

### Intermediate 1

### a) 2-(Benzyloxy)-4,6-dimethylnicotinonitrile

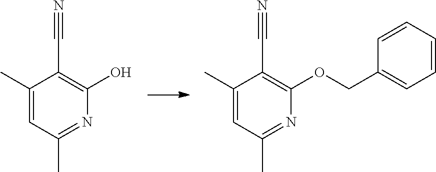 [SMILES: Cc1cc(C)c(C#N)c(O)n1 ; Cc1cc(C)c(C#N)c(OCc2ccccc2)n1]

A solution of 2-hydroxy-4,6-dimethylnicotinonitrile (5 g, 33.7 mmol) in toluene (50 mL) was treated with benzyl chloride (4.70 mL, 40.5 mmol) and silver oxide (8.60 g, 37.1 mmol), then stirred at 110° C. overnight. The reaction was filtered through Celite® and the solids were washed with DCM (2×100 mL). The combined organic layers were washed with brine (30 mL), filtered through Na₂SO₄ and concentrated in vacuo to give a residue. The residue was purified through a plug of silica with vacuum using 20-30% DCM in petroleum ether. The desired fractions were combined and concentrated to furnish 2-(benzyloxy)-4,6-dimethylnicotinonitrile (9 g, 35.9 mmol, 106% yield) as a white solid. ¹H NMR (400 MHz, CDCl₃) δ 7.47-7.56 (m, 2H), 7.31-7.43 (m, 3H), 6.72 (s, 1H), 5.51 (s, 2H), 2.48 (d, J=3.03 Hz, 6H). MS(ES) [M+H]⁺ 239.0.

### b) 2-(Benzyloxy)-4,6-dimethylnicotinaldehyde

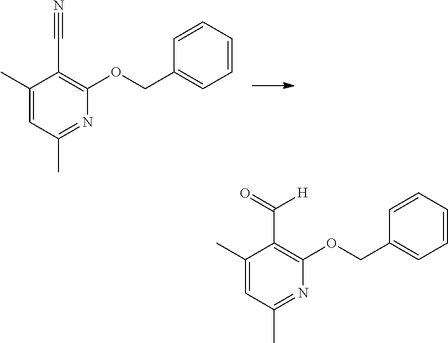 [SMILES: Cc1cc(C)c(C#N)c(OCc2ccccc2)n1 ; Cc1cc(C)c(C=O)c(OCc2ccccc2)n1]

To a cooled (ice bath) solution of 2-(benzyloxy)-4,6-dimethylnicotinonitrile (9 g, 35.9 mmol) in DCM (100 mL) under an inert atmosphere was slowly added a solution of 1 M DIBAL-H in toluene (43.1 mL, 43.1 mmol) via syringe. The reaction was stirred at 0° C. for 20 min, at which time the ice-bath was removed and the reaction stirred at RT overnight. LCMS showed ˜14% starting material remained. An additional portion of 1 M DIBAL-H in toluene (10.76 mL, 10.76 mmol) was added and the reaction continued to stir at RT. LCMS indicated the reaction was complete. The reaction was cooled (ice bath) and quenched with 1N HCl (50 mL). **Caution—exothermic. The reaction was stirred 30 min until the aluminum salts were free flowing. The reaction was neutralized with 2.5 N NaOH (˜15 mL, ˜pH7.5). The biphasic mixture was filtered and the filtrate washed with DCM (100 mL, 2×). The layers were separated and the aqueous was extracted with DCM (100 mL). The combined organic layers were washed with brine (30 mL), filtered through Na₂SO₄ and concentrated in vacuo. The residue was purified by flash chromatography (Column: 80 gram silica. Eluent: 0-5% EtOAc in Heptanes. Gradient: 15 min). The desired fractions were combined and concentrated in vacuo to give 2-(benzyloxy)-4,6-dimethylnicotinaldehyde (3.5 g, 14.36 mmol, 40.0% yield) as a fluffy white solid. ¹H NMR (400 MHz, CDCl₃) δ 10.58 (s, 1H), 7.49 (d, J=7.07 Hz, 2H), 7.31-7.44 (m, 3H), 6.67 (s, 1H), 5.54 (s, 2H), 2.59 (s, 3H), 2.50 (s, 3H). MS(ES) [M+H]⁺ 242.1, [M+Na]⁺ 264.0.

### c) (2-(Benzyloxy)-4,6-dimethylpyridin-3-yl)methanol

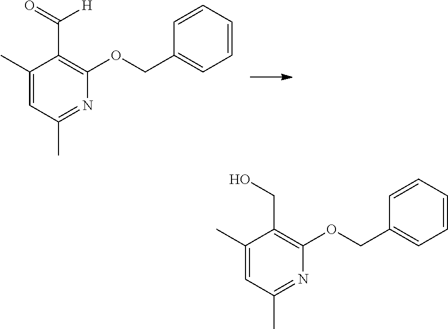 [SMILES: Cc1cc(C)c(C=O)c(OCc2ccccc2)n1 ; Cc1cc(C)c(CO)c(OCc2ccccc2)n1]

A suspension of 2-(benzyloxy)-4,6-dimethylnicotinaldehyde (3.46 g, 14.34 mmol) in MeOH (100 mL) was kept under inert atmosphere and cooled to 0° C. in an ice bath. To the stirred suspension was added NaBH₄ (0.651 g, 17.21 mmol) in two portions. The suspension went into solution after the first portion of NaBH₄ was added. The reaction was stirred at 0° C. for 10 min, at which time the ice-bath was removed and the reaction stirred at RT overnight. The reaction solvent was removed in vacuo and the remaining white solid residue was partitioned between saturated NaHCO₃ (60 mL) and EtOAc (125 mL). The aqueous layer was extracted with EtOAc (125 mL). The combined organic layers were washed with brine (20 mL), filtered through Na₂SO₄ and concentrated in vacuo to give (2-(benzyloxy)-4,6-dimethylpyridin-3-yl)methanol (3.5 g, 14.39 mmol, 100% yield) as a colorless translucent oil. ¹H NMR (400 MHz, CDCl₃) δ 7.44-7.53 (m, 2H), 7.30-7.43 (m, 3H), 6.63 (s, 1H), 5.46 (s, 2H), 4.72 (s, 2H), 2.43 (s, 3H), 2.35 (s, 3H), 2.25 (br. s., 1H). MS(ES) [M+H]⁺ 244.1.

### d) 2-(Benzyloxy)-3-(chloromethyl)-4,6-dimethylpyridine

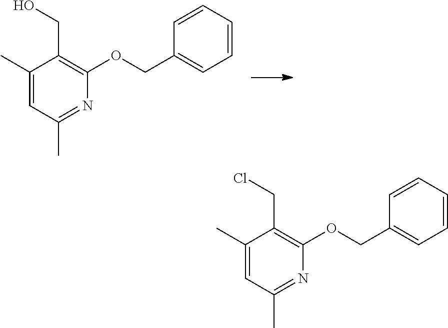 [SMILES: Cc1cc(C)c(CO)c(OCc2ccccc2)n1 ; Cc1cc(C)c(CCl)c(OCc2ccccc2)n1]

A suspension of (2-(benzyloxy)-4,6-dimethylpyridin-3-yl)methanol (3.5 g, 14.39 mmol) in DCM (70 mL) was kept under inert atmosphere and cooled to −40° C. in dry ice/CH₃CN bath for 30 min. To the chilled solution was added 2 M SOCl₂ in DCM (10.79 mL, 21.58 mmol) in one portion and the reaction continued to stir at −40° C. After 1 h, LCMS showed 5% starting material remained. Additional 2 M SOCl₂ in DCM (1.439 mL, 2.88 mmol) was added and the reaction continued. After 20 min, the reaction was poured into ice water and the pH was adjusted to 7-8 with saturated NaHCO₃ (30 mL). The aqueous layer was extracted with DCM (125 mL, 2×). The combined organic layers were washed with brine (50 mL), filtered through Na₂SO₄ and concentrated in vacuo. The residue was purified by flash chromatography (Column: 80 grams silica. Eluent: 0-10% EtOAc/Heptanes. Gradient: 14 min) to give 2-(benzyloxy)-3-(chloromethyl)-4,6-dimethylpyridine (2.84 g, 10.74 mmol, 74.7% yield) as a colorless oil. ¹H NMR (400 MHz, CDCl₃) δ 7.48-7.57 (m, 2H), 7.30-7.45 (m, 3H), 6.64 (s, 1H), 5.47 (s, 2H), 4.74 (s, 2H), 2.43 (s, 3H), 2.38 (s, 3H). MS(ES) [M+H]⁺ 262.1.

### Intermediate 2

### a) Methyl 4-methylthiophene-3-carboxylate

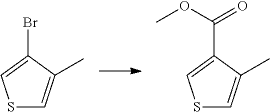

To a stirred solution of 3-bromo-4-methylthiophene (20.0 g, 113 mmol) in THF (100 mL) under nitrogen at RT was added isopropylmagnesium chloride lithium chloride complex 1.3 N in THF (90 mL, 117 mmol) dropwise. The reaction was stirred overnight. The reaction was cooled to −78° C. and treated with methyl chloroformate (12 mL, 155 mmol). The reaction was allowed to warm to RT and stirred for 1 hr. The reaction was diluted with EtOAc, washed with saturated NaHCO₃, stirred for 30 min, (formed a white suspension that stayed in the aqueous phase), washed with brine, dried (Na₂SO₄), filtered and concentrated under vacuum. The product was short path distilled under vacuum (4 to 2 mm Hg) at 44 to 50° C. (oil bath 50 to 75° C.). The main and late fractions were combined to give the product methyl 4-methylthiophene-3-carboxylate (13.2 g, 85 mmol, 74.8% yield) as a clear liquid. MS(ES) [M+H]⁺ 156.8.

### b) Methyl 4-methyl-5-propionylthiophene-3-carboxylate

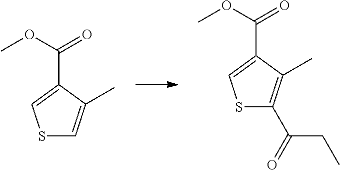

To a stirred solution of methyl 4-methylthiophene-3-carboxylate (5.0 g, 32.0 mmol) in CH₃NO₂ (50 mL) was added LiClO₄ (4.0 g, 37.6 mmol), propionic anhydride (5.87 mL, 38.4 mmol) and In(OTf)₃ (0.9 g, 1.601 mmol). The reaction was stirred at 50° C. for 2 hr. LCMS showed that the reaction was complete. The reaction was diluted with water (100 mL), extracted with DCM (2×50 mL), dried (Na₂SO₄), filtered and evaporated to dryness under vacuum. The remaining brown solid was purified by silica gel chromatography (Isco RediSep® Rf Gold 120 g, 0 to 25% EtOAc in hexanes) (loaded with DCM). The pure fractions were combined and evaporated to dryness. The remaining light yellow solid was triturated with hexanes, filtered and dried under vacuum to give the product methyl 4-methyl-5-propionylthiophene-3-carboxylate (4.60 g, 21.67 mmol, 67.7% yield) as a white solid. MS(ES) [M+H]⁺ 212.9.

### Intermediate 3

### Methyl 5-bromo-4-methylthiophene-3-carboxylate

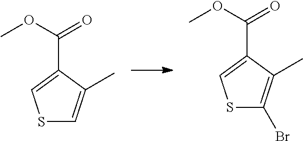

To a solution of methyl 4-methylthiophene-3-carboxylate (12 g, 77 mmol) in DMF (200 mL) was added NBS (14.36 g, 81 mmol). The reaction was stirred at RT overnight. The mixture was poured into water (1.5 L), stirred for 1 h, and filtered. Methyl 5-bromo-4-methylthiophene-3-carboxylate (17.5 g, 70.7 mmol, 92% yield) was isolated as white solid (melted upon drying in vac oven, solidified upon freezer storage).

### Intermediate 4

### 2-Fluoro-2-methylpropyl trifluoromethanesulfonate

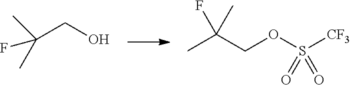

To a cooled (−20° C.) solution of 2-fluoro-2-methylpropan-1-ol (1.3 g, 14.11 mmol), Et₃N (2.361 mL, 16.94 mmol) and DMAP (0.121 g, 0.988 mmol) in DCM (8 mL) was added dropwise Tf₂O (2.86 mL, 16.94 mmol). The reaction was stirred at 0° C. for 2 h.

The reaction was diluted with DCM, washed with 1 M aqueous citric acid, and saturated NaHCO₃. The organic layer was dried (Na₂SO₄) and concentrated to give 2.2 g of 2-fluoro-2-methylpropyl trifluoromethanesulfonate as a brown oil.

### EXAMPLES

### Example 1

### 5-(1-(1-(2,2-Difluoropropyl)piperidin-4-ylidene)propyl)-N-((4,6-dimethyl-2-oxo-1,2-dihydropyridin-3-yl)methyl)-4-methylthiophene-3-carboxamide

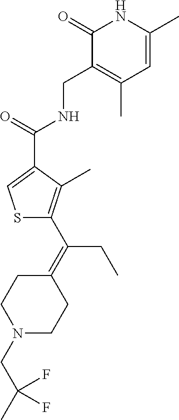 [SMILES: CCC(=C1CCN(CC(C)(F)F)CC1)c1scc(C(=O)NCc2c(C)cc(C)[nH]c2=O)c1C]

### a) tert-Butyl 4-(1-(4-(methoxycarbonyl)-3-methylthiophen-2-yl)propylidene)piperidine-1-carboxylate

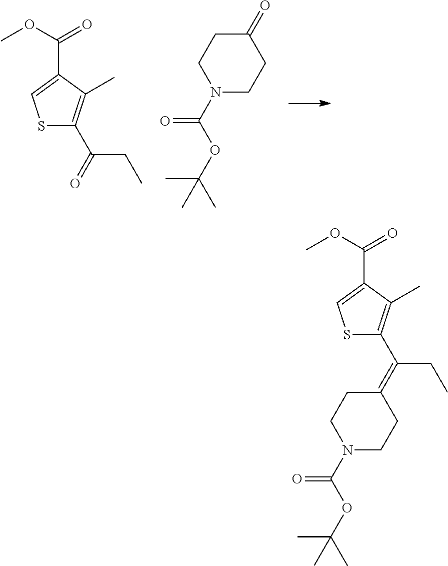

To a stirred suspension of zinc powder (21 g, 321 mmol) and THF (250 mL) in a 1 L round bottom flask, under nitrogen, with cooling to 0° C. in an ice bath was added TiCl₄ (17 mL, 155 mmol) via syringe through a short condensor (vigorous reaction with yellow fumes). The reaction was rinsed down with THF (75 mL) and the resulting black slurry was heated at reflux (70° C. oil bath) for 2 h (the reaction stopped stirring, but resumed as it warmed). A solution of methyl 4-methyl-5-propionylthiophene-3-carboxylate (5.0 g, 23.56 mmol), tert-butyl 4-oxopiperidine-1-carboxylate (15.0 g, 75 mmol) and pyridine (20 mL, 247 mmol) in THF (50 mL) was added and the heating was continued for 2 days. The reaction was allowed to cool to RT, quenched with sat. aq. NH₄Cl (500 mL), and extracted with EtOAc (200 mL). The upper EtOAc phase was decanted off carefully onto a pad of Celite® and filtered. This protocol was repeated three times by stirring the dark blue aqueous suspension with fresh EtOAc and decanting (Note: the lower dark aqueous suspension could not be easily filtered and eventually plugged the filter). The combined organics were washed with brine, dried (Na₂SO₄), filtered and evaporated to dryness.

The crude amine hydrochloride above was taken up in DCM (250 mL) and treated at 0° C. in an ice bath with Et₃N (6.0 mL, 43.0 mmol) and Boc₂O (9.57 mL, 41.2 mmol). The reaction was allowed to warm to RT and was stirred for 1 h. The reaction was concentrated under vacuum, taken up in EtOAc and washed with aq. NaHCO₃ (large volume of white solid formed). The solids were filtered off and rinsed with EtOAc. The clear filtrate containing the product was transferred to a separatory funnel. The lower NaHCO₃ phase was removed and the EtOAc phase washed with 1N HCl, brine, dried (Na₂SO₄), filtered and evaporated to dryness. The residue was purified by silica gel chromatography (Isco RediSep® Rf Gold 120 g, 0 to 25% EtOAc in hexanes). The pure fractions were combined and evaporated to dryness to give tert-butyl 4-(1-(4-(methoxycarbonyl)-3-methylthiophen-2-yl)propylidene)piperidine-1-carboxylate (6.08 g, 14.10 mmol, 59.9% yield) as a colorless oil, which solidified to a white solid under vacuum. The reaction was repeated a second time to give a total of 12.17 g product. ¹H NMR (400 MHz, CDCl₃) δ 8.04 (s, 1H), 3.86 (s, 3H), 3.51 (br. s., 2H), 3.34 (br. s., 2H), 2.44 (t, J=5.8 Hz, 2H), 2.34 (br. s., 2H), 2.24 (s, 3H), 2.01 (t, J=5.8 Hz, 2H), 1.48 (s, 9H), 0.94 (t, J=7.6 Hz, 3H). MS(ES) [M+H]⁺-Boc 280.0, [M+H]⁺-isobutylene 324.1, M+Na⁺ 402.1.

### b) Methyl 5-(1-(1-(2,2-difluoropropyl)piperidin-4-ylidene)propyl)-4-methylthiophene-3-carboxylate

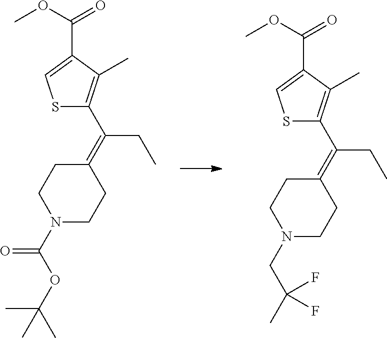 [SMILES: CCC(=C1CCN(C(=O)OC(C)(C)C)CC1)c1scc(C(=O)OC)c1C ; CCC(=C1CCN(CC(C)(F)F)CC1)c1scc(C(=O)OC)c1C]

To tert-butyl 4-(1-(4-(methoxycarbonyl)-3-methylthiophen-2-yl)propylidene)piperidine-1-carboxylate (12.1 g, 31.9 mmol) was added HCl in dioxane (30 mL, 120 mmol). After stirring for 30 min the reaction was evaporated to dryness to give the crude amine hydrochloride salt as a white solid foam.

To a stirred solution of 2,2-difluoropropan-1-ol (16.3 g, 170 mmol) and pyridine (16.3 mL, 202 mmol) in CH₃CN (250 mL) at 0° C. in an ice bath was added dropwise Tf₂O (28 mL, 166 mmol). The reaction was stirred for 30 min at 0° C., then added cold to a slurry of the above amine hydrochloride and K₂CO₃ (46.8 g, 339 mmol) in CH₃CN (100 mL). The reaction was rinsed down with CH₃CN (50 mL). The reaction was allowed to warm to RT, heated to 50° C. and stirred for 6 h. The reaction was evaporated to dryness under vacuum, taken up in DCM, washed with water, brine, dried (Na₂SO₄), filtered, and concentrated under vacuum. The residue was purified by silica gel chromatography (Isco RediSep® Rf Gold 120 g, 5 to 15% EtOAc in hexanes). The pure fractions were combined and evaporated to dryness under vacuum to give methyl 5-(1-(1-(2,2-difluoropropyl)piperidin-4-ylidene)propyl)-4-methylthiophene-3-carboxylate (10.05 g, 24.74 mmol, 78% yield) as a colorless oil. ¹H NMR (400 MHz, CDCl₃) δ 8.03 (s, 1H), 3.86 (s, 3H), 2.70 (t, J=12.6 Hz, 4H), 2.57-2.43 (m, 4H), 2.32 (br. s., 2H), 2.25 (s, 3H), 2.06 (br. s., 2H), 1.67 (t, J=18.8 Hz, 3H), 0.94 (t, J=7.5 Hz, 3H). MS(ES) [M+H]⁺ 358.2.

### c) 5-(1-(1-(2,2-Difluoropropyl)piperidin-4-ylidene)propyl)-N-((4,6-dimethyl-2-oxo-1,2-dihydropyridin-3-yl)methyl)-4-methylthiophene-3-carboxamide

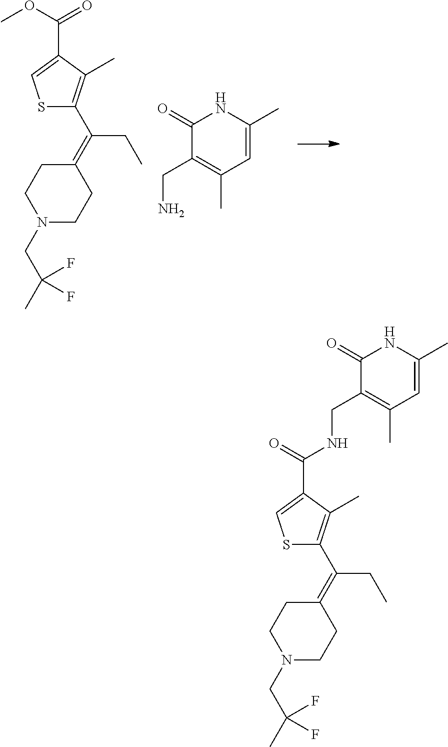

To a stirred solution of methyl 5-(1-(1-(2,2-difluoropropyl)piperidin-4-ylidene)propyl)-4-methylthiophene-3-carboxylate (10.0 g, 28.0 mmol) in MeOH (150 mL) was added 5 N NaOH (20 mL, 100 mmol). The reaction was stirred at 70° C. for 4 h. The reaction was concentrated under vacuum to remove the MeOH and neutralized with 6 N HCl (16.7 mL) to pH-7. A gummy mass formed that was extracted with DCM, dried (Na₂SO₄), filtered and evaporated to a solid foam.

To the above was added 3-(aminomethyl)-4,6-dimethylpyridin-2(1H)-one hydrochloride (5.8 g, 30.7 mmol), HOAt (3.8 g, 27.9 mmol), DCM (150 mL) and NMM (3.4 mL, 30.9 mmol). Any solid clumps were broken up with a stir rod. To the stirring suspension was added EDC free base (5.0 g, 32.2 mmol). The reaction was stirred at RT for 3 h then overnight at 40° C. with a reflux condensor attached. The reaction was concentrated under vacuum. The cloudy solution was filtered through a pad of Celite® and rinsed with a small volume of DCM. The clear filtrate was concentrated and purified by silica gel chromatography (Isco RediSep® Rf Gold 220 g, 0 to 5% EtOH in EtOAc). The pure fractions were combined and evaporated to dryness under vacuum. The resultant solid was triturated with 10% EtOAc/hexanes, filtered, washed with hexanes and dried under vacuum to give 5-(1-(1-(2,2-difluoropropyl)piperidin-4-ylidene)propyl)-N-((4,6-dimethyl-2-oxo-1,2-dihydropyridin-3-yl)methyl)-4-methylthiophene-3-carboxamide (11.56 g, 24.20 mmol, 87% yield) as a white solid. ¹H NMR (400 MHz, DMSO-d₆) δ 11.48 (s, 1H), 8.01 (t, J=5.1 Hz, 1H), 7.78 (s, 1H), 5.86 (s, 1H), 4.24 (d, J=5.1 Hz, 2H), 2.71 (t, J=14.1 Hz, 2H), 2.61 (t, J=4.7 Hz, 2H), 2.46 (br. s., 2H), 2.38 (t, J=5.6 Hz, 2H), 2.30-2.21 (m, 2H), 2.18 (s, 3H), 2.11 (s, 3H), 2.07 (s, 3H), 1.93 (t, J=5.3 Hz, 2H), 1.62 (t, J=19.2 Hz, 3H), 0.86 (t, J=7.5 Hz, 3H). MS(ES) [M+H]⁺ 478.3.

### Example 2

### N-((4,6-Dimethyl-2-oxo-1,2-dihydropyridin-3-yl)methyl)-5-(1-(1-(2-fluoro-2-methylpropyl)piperidin-4-ylidene)propyl)-4-methylthiophene-3-carboxamide

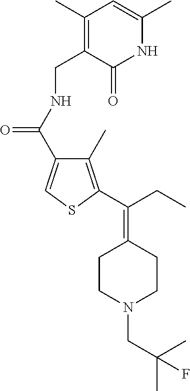 [SMILES: CCC(=C1CCN(CC(C)(C)F)CC1)c1scc(C(=O)NCc2c(C)cc(C)[nH]c2=O)c1C]

### a) Methyl 5-(1-(1-(2-fluoro-2-methylpropyl)piperidin-4-ylidene)propyl)-4-methylthiophene-3-carboxylate

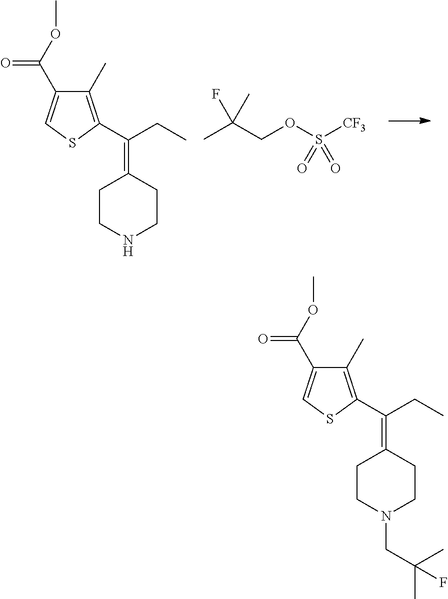

To a solution of methyl 4-methyl-5-(1-(piperidin-4-ylidene)propyl)thiophene-3-carboxylate hydrochloride (160 mg, 0.507 mmol) in CH₃CN (5 mL) was added Cs₂CO₃ (330 mg, 1.013 mmol) and 2-fluoro-2-methylpropyl trifluoromethanesulfonate (454 mg, 2.026 mmol). The mixture was heated at 50° C. for 1 8 h. The reaction mixture was cooled to RT, quenched with water and extracted with EtOAc (3×). The combined organics were dried (Na₂SO₄) and concentrated. The residue was purified using column chromatography (silica gel, 0 to 50% EtOAc/hexanes) to give methyl 5-(1-(1-(2-fluoro-2-methylpropyl)piperidin-4-ylidene)propyl)-4-methylthiophene-3-carboxylate (92 mg) as a colorless oil. MS(ES) [M+H]⁺ 354.3.

### b) N-((4,6-Dimethyl-2-oxo-1,2-dihydropyridin-3-yl)methyl)-5-(1-(1-(2-fluoro-2-methylpropyl)piperidin-4-ylidene)propyl)-4-methylthiophene-3-carboxamide

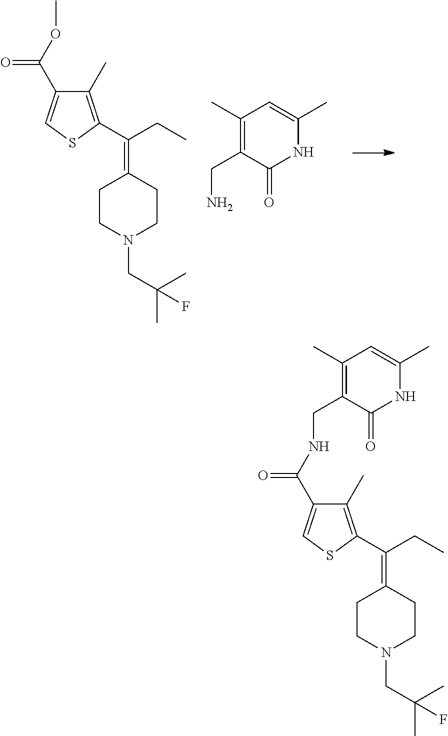

To a solution of methyl 5-(1-(1-(2-fluoro-2-methylpropyl)piperidin-4-ylidene)propyl)-4-methylthiophene-3-carboxylate (90 mg, 0.255 mmol) in MeOH (2 mL) was added 8 N NaOH (0.159 mL, 1.273 mmol). The mixture was heated at 35° C. for 18 h. The mixture was neutralized with 6 N HCl (0.212 mL, 1.273 mmol), concentrated.

To a solution of the residue in dimethyl sulfoxide (2.000 mL) were added 3-(aminomethyl)-4,6-dimethylpyridin-2(1H)-one hydrochloride (62.4 mg, 0.331 mmol), NMM (0.140 mL, 1.273 mmol), EDC (98 mg, 0.509 mmol) and HOAt (69.3 mg, 0.509 mmol). The mixture was stirred at rt for 18 h. The mixture was quenched with water (10 mL). The resulting precipitate was collected by filtration, washed with water and dried under vacuum to give N-((4,6-dimethyl-2-oxo-1,2-dihydropyridin-3-yl)methyl)-5-(1-(1-(2-fluoro-2-methylpropyl)piperidin-4-ylidene)propyl)-4-methylthiophene-3-carboxamide (105 mg) as an off-white solid. ¹H NMR (400 MHz, DMSO-d₆) δ 0.86 (t, J=7.45 Hz, 3H), 1.28 (s, 3H), 1.33 (s, 3H), 1.93 (t, J=5.31 Hz, 2H), 2.07 (s, 3H), 2.11 (s, 3H), 2.18 (s, 3H), 2.20-2.32 (m, 2H), 2.34-2.46 (m, 6H), 4.23 (d, J=5.05 Hz, 2H), 5.86 (s, 1H), 7.78 (s, 1H), 8.01 (t, J=4.93 Hz, 1H). MS(ES) [M+H]⁺ 474.3.

### Example 3

### N-((4,6-Dimethyl-2-oxo-1,2-dihydropyridin-3-yl)methyl)-4-methyl-5-(1-(1-(2,2,2-trifluoroethyl)piperidin-4-ylidene)propyl)thiophene-3-carboxamide

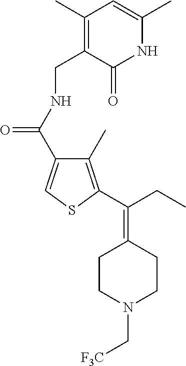 [SMILES: CCC(=C1CCN(CC(F)(F)F)CC1)c1scc(C(=O)NCc2c(C)cc(C)[nH]c2=O)c1C]

Following the general procedure of Example 2, N-((4,6-dimethyl-2-oxo-1,2-dihydropyridin-3-yl)methyl)-4-methyl-5-(1-(1-(2,2,2-trifluoroethyl)piperidin-4-ylidene)propyl)thiophene-3-carboxamide was prepared. ¹H NMR (400 MHz, DMSO-d₆) δ 11.48 (s, 1H), 8.01 (t, J=4.93 Hz, 1H), 7.79 (s, 1H), 5.86 (s, 1H), 4.24 (d, J=5.05 Hz, 2H), 3.33 (s, 2H), 3.18 (q, J=10.27 Hz, 2H), 2.69 (d, J=5.05 Hz, 2H), 2.39 (t, J=5.56 Hz, 2H), 2.26 (d, J=6.82 Hz, 2H), 2.18 (s, 3H), 2.11 (s, 3H), 2.07 (s, 3H), 1.93 (t, J=5.31 Hz, 2H), 0.87 (t, J=7.45 Hz, 3H). MS(ES) [M+H]⁺ 482.3.

### Example 4

### (R)-5-(1-(1-(2,2-Difluoropropyl)piperidin-4-yl)propyl)-N-((4,6-dimethyl-2-oxo-1,2-dihydropyridin-3-yl)methyl)-4-methylthiophene-3-carboxamide

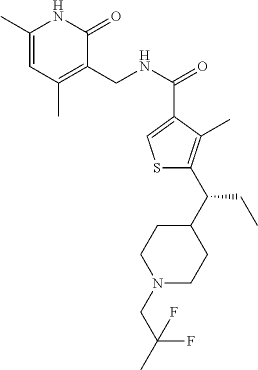 [SMILES: CC[C@@H](c1scc(C(=O)NCc2c(C)cc(C)[nH]c2=O)c1C)C1CCN(CC(C)(F)F)CC1]

### a) tert-Butyl 4-propionylpiperidine-1-carboxylate

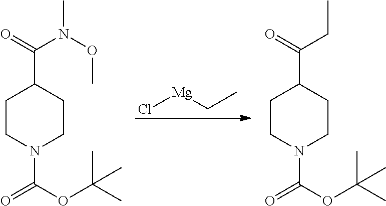

To a stirred solution of tert-butyl 4-(methoxy(methyl)carbamoyl)piperidine-1-carboxylate (10.0 g, 36.7 mmol) in THF (100 mL) at 0° C. (ice bath) under nitrogen was added dropwise 2 N ethylmagnesium chloride in THF (28 mL, 56.0 mmol). The reaction was stirred at 0° C. for 4 h, then quenched with saturated NH₄Cl, extracted with EtOAc, washed with brine, dried (Na₂SO₄), filtered and evaporated to dryness under vacuum. The crude product was purified by silica gel chromatography (Isco RediSep® Rf Gold 220 g, 0 to 40% EtOAc in hexanes). (UV negative, visualized by charring with H₂SO₄ in EtOH.) The pure fractions were combined and evaporated to dryness to give tert-butyl 4-propionylpiperidine-1-carboxylate (8.10 g, 33.6 mmol, 91% yield) as a colorless oil. MS(ES) [M+H]+-isobutylene-18 167.9, [M+H]+-isobutylene 186.0, M+Na+ 264.1.

### b) (Z)-tert-Butyl 4-(1-(((trifluoromethyl)sulfonyl)oxy)prop-1-en-1-yl)piperidine-1-carboxylate

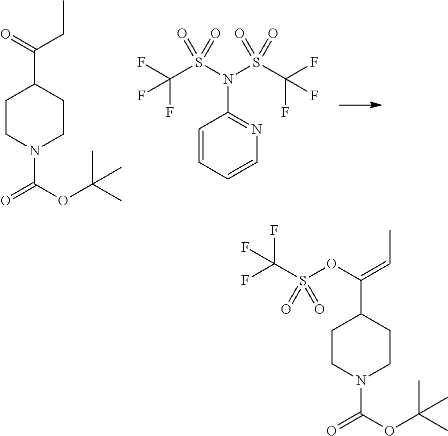 [SMILES: CCC(=O)C1CCN(C(=O)OC(C)(C)C)CC1 ; O=S(=O)(N(c1ccccn1)S(=O)(=O)C(F)(F)F)C(F)(F)F ; C/C=C(\OS(=O)(=O)C(F)(F)F)C1CCN(C(=O)OC(C)(C)C)CC1]

To a stirred solution of tert-butyl 4-propionylpiperidine-1-carboxylate (6.9 g, 28.6 mmol) in THF (80 mL) at −78° C. (CO₂, acetone) under nitrogen was added dropwise 1 N NaHMDS in THF (31 mL, 31.0 mmol). The reaction was stirred at −78° C. for 1 h. A solution of 2-NTf₂-pyridine (11.4 g, 31.8 mmol) in THF (50 mL) was next added dropwise over 5 min. The reaction was stirred for 1 h at −78° C., then at 0° C. for 30 min. The reaction was quenched with water (150 mL), extracted with EtOAc (2×150 mL), washed with brine, dried (Na₂SO₄), filtered and concentrated under vacuum. The crude product was purified by silica gel chromatography (Isco RediSep® Rf Gold 220 g, 0 to 20% EtOAc in hexanes). (UV negative, visualized by charring with H₂SO₄ in EtOH.) The pure fractions were combined and evaporated to dryness to give (Z)-tert-butyl 4-(1-(((trifluoromethyl)sulfonyl)oxy)prop-1-en-1-yl)piperidine-1-carboxylate (9.15 g, 24.51 mmol, 86% yield) as a colorless oil. MS(ES) [M+H]+-isobutylene 318.1.

### c) (Z)-tert-Butyl 4-(1-(4-(methoxycarbonyl)-3-methylthiophen-2-yl)prop-1-en-1-yl)piperidine-1-carboxylate

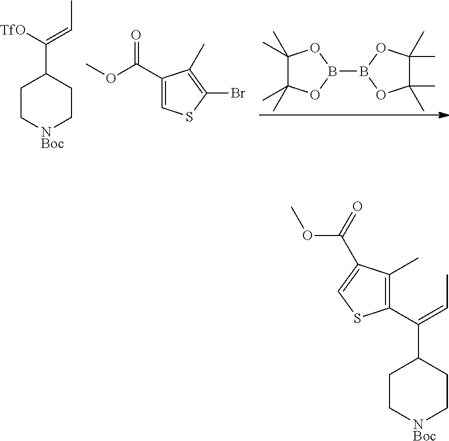

To a degassed solution of 4,4,4′,4′,5,5,5′,5′-octamethyl-2,2′-bi(1,3,2-dioxaborolane) (17.39 g, 68.5 mmol), methyl 5-bromo-4-methylthiophene-3-carboxylate (7 g, 29.8 mmol) and KOAc (9.64 g, 98 mmol) in 1,4-dioxane (200 mL) was added PdCl₂(dppf)-DCM adduct (1.216 g, 1.489 mmol). The reaction mixture was heated overnight at 70° C., at which time the suspension was filtered through a short pad of silica. To the filtrate was added (E)-tert-butyl 4-(1-(((trifluoromethyl)sulfonyl)oxy)prop-1-en-1-yl)piperidine-1-carboxylate (6.67 g, 17.86 mmol), water (60 mL) and Na₂CO₃ (7.89 g, 74.4 mmol). The solution was degassed, and Pd(PPh₃)₄ (1.720 g, 1.489 mmol) was added. The reaction mixture was heated at 70° C. for 1 h. The reaction was diluted with EtOAc (200 mL) and filtered. The layers were separated and the organics were washed with brine, dried over Mg₂SO₄, filtered and evaporated. The residue was purified by flash chromatography (8% THF:hexanes) to give (Z)-tert-butyl 4-(1-(4-(methoxycarbonyl)-3-methylthiophen-2-yl)prop-1-en-1-yl)piperidine-1-carboxylate (6.2 g, 15.52 mmol, 52.1% yield) as white solid. MS(ES) [M+H]′ 402.2 (M+Na)

### d) Methyl 4-methyl-5-(1-(piperidin-4-yl)propyl)thiophene-3-carboxylate

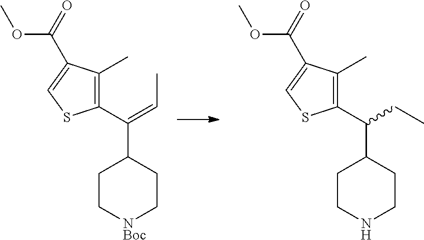 [SMILES: C/C=C(\c1scc(C(=O)OC)c1C)C1CCN(C(=O)OC(C)(C)C)CC1 ; CCC(c1scc(C(=O)OC)c1C)C1CCNCC1]

To a solution of (Z)-tert-butyl 4-(1-(4-(methoxycarbonyl)-3-methylthiophen-2-yl)prop-1-en-1-yl)piperidine-1-carboxylate (6.2 g, 16.34 mmol) in EtOH (120 mL) was added 10% Pd/C (Degussa, 12 g, 11.28 mmol). The reaction was stirred under a H₂ atmosphere (balloon) for 24 h, at which time the mixture was filtered through Celite® and evaporated. The residue was dissolved in dioxane (10 mL) and 3 M HCl (10 mL) was added. The reaction mixture was heated at reflux for 10 min, then evaporated. The residue was partitioned between EtOAc (100 mL) and 1 M Na₂CO₃ (50 mL). The layers were separated and the organics were washed with brine, dried over MgSO₄, filtered and evaporated to give tert-butyl 4-(1-(4-(methoxycarbonyl)-3-methylthiophen-2-yl)propyl)piperidine-1-carboxylate (2.6 g, 8.78 mmol, 53.7% yield) as a colorless liquid. MS(ES) [M+H]′ 282.2.

### e) (S)-methyl 5-(1-(1-(2,2-difluoropropyl)piperidin-4-yl)propyl)-4-methylthiophene-3-carboxylate and (R)-methyl 5-(1-(1-(2,2-difluoropropyl)piperidin-4-yl)propyl)-4-methylthiophene-3-carboxylate

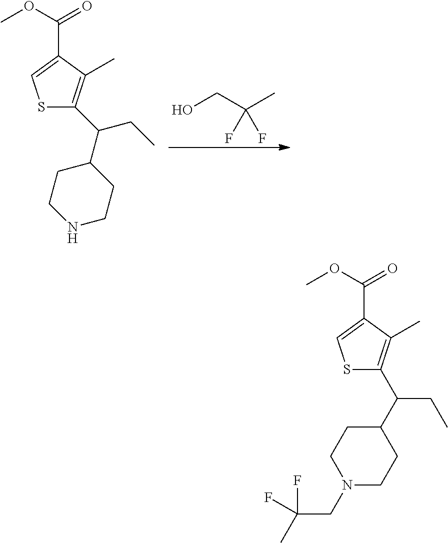

To a cooled (0° C.) solution of 2,2-difluoropropan-1-ol (3.17 g, 33.0 mmol) and pyridine (2.97 mL, 36.7 mmol) in CH₃CN (100 mL) was added dropwise Tf₂O (5.70 mL, 33.8 mmol). The reaction was stirred for 30 min at 0° C. To the cooled slurry was added a cold solution of methyl 4-methyl-5-(1-(piperidin-4-yl)propyl)thiophene-3-carboxylate hydrochloride (2.6 g, 7.34 mmol) and K₂CO₃ (9.13 g, 66.0 mmol) in CH₃CN (20 mL). The reaction was allowed to warm to RT, then heated at 50° C. overnight. The reaction was evaporated to dryness under vacuum, taken up in DCM, washed with water, brine, dried (Na₂SO₄), filtered, and concentrated under vacuum. The residue was purified by silica gel chromatography (Isco RediSep® Rf Gold 120 g, 5% EtOAc:hexanes) to give methyl 5-(1-(1-(2,2-difluoropropyl)piperidin-4-yl)propyl)-4-methylthiophene-3-carboxylate (2.05 g, 5.42 mmol, 73.8% yield) as a yellow oil. ¹H NMR (400 MHz, CDCl₃) δ 8.00 (s, 1H), 3.85 (s, 3H), 2.99 (d, J=11.12 Hz, 1H), 2.86 (d, J=11.12 Hz, 1H), 2.53-2.75 (m, 3H), 2.37 (s, 3H), 2.04-2.27 (m, 2H), 1.84-2.01 (m, 2H), 1.62 (t, J=18.69 Hz, 4H), 1.30-1.47 (m, 5H), 0.76 (t, J=7.33 Hz, 3H). MS(ES) [M+H]⁺ 360.2.

The racemic product was resolved by chiral HPLC (Chiralcel OD-H, 5 microns, 30 mm×250 mm, 250 nm UV, 98:2:0.1 n-heptane:2-propanol:isopropylamine). The resolved products were twice diluted with 2-propanol and concentrated, then dried in a vac oven (50° C.) to give:

S-(−)-methyl 5-(1-(1-(2,2-difluoropropyl)piperidin-4-yl)propyl)-4-methylthiophene-3-carboxylate (870 mg): >99.8% ee, [α]_{D}=−9.6° (c=0.50, MeOH, 24° C.) and

R-(+)-methyl 5-(1-(1-(2,2-difluoropropyl)piperidin-4-yl)propyl)-4-methylthiophene-3-carboxylate (860 mg): 99.74% ee; [α]_{D}=+8.8° (c=0.50, MeOH, 24° C.).

### f) (R)-5-(1-(1-(2,2-Difluoropropyl)piperidin-4-yl)propyl)-N-((4,6-dimethyl-2-oxo-1,2-dihydropyridin-3-yl)methyl)-4-methylthiophene-3-carboxamide

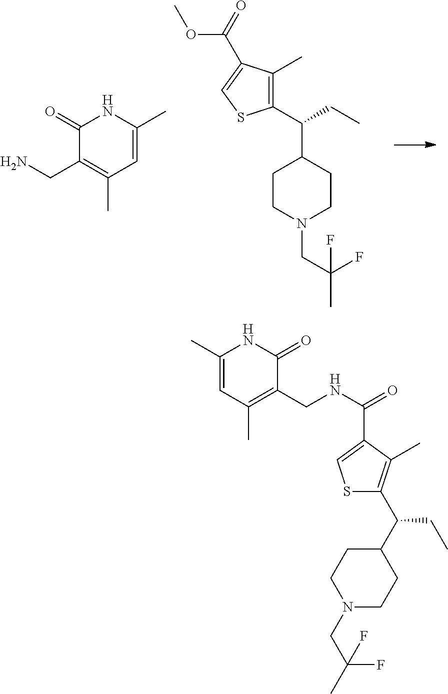

To a solution of (R)-methyl 5-(1-(1-(2,2-difluoropropyl)piperidin-4-yl)propyl)-4-methylthiophene-3-carboxylate (860 mg, 2.392 mmol) in MeOH (10 mL) was added 3 M NaOH (5 mL, 400 mmol). The reaction mixture was stirred overnight, at which time it was neutralized with 6 M HCl and evaporated to dryness.

To a suspension of the residue in DMF (10.00 mL) was added 3-(aminomethyl)-4,6-dimethylpyridin-2(1H)-one hydrochloride (542 mg, 2.87 mmol), followed by EDC (550 mg, 2.87 mmol) and HOAt (391 mg, 2.87 mmol). After 5 min, NMM (0.789 mL, 7.18 mmol) was added and the solution was stirred for 3 h at RT. The reaction mixture was poured into 60 mL of water and extracted with EtOAc (2×50 mL). The combined organics were washed with water (30 mL), brine, dried over MgSO₄, filtered and evaporated. The residue was crystalized from 20% CH₃CN:water to give (R)-5-(1-(1-(2,2-difluoropropyl)piperidin-4-yl)propyl)-N-((4,6-dimethyl-2-oxo-1,2-dihydropyridin-3-yl)methyl)-4-methylthiophene-3-carboxamide (780 mg, 1.626 mmol, 68.0% yield) as white solid (Note: the absolute stereochemistry of the ethyl group was assigned based on a known preference for R-isomer with regards to EZH2 inhibition). ¹H NMR (400 MHz, MeOH-d₄) δ 7.60 (s, 1H), 6.12 (s, 1H), 4.45 (s, 2H), 3.01 (d, J=11.62 Hz, 1H), 2.89 (d, J=11.12 Hz, 1H), 2.74 (ddd, J=3.79, 7.89, 11.05 Hz, 1H), 2.64 (t, J=14.02 Hz, 2H), 2.37 (s, 2H), 2.25 (d, J=5.56 Hz, 6H), 2.18 (dt, J=2.40, 11.56 Hz, 1H), 2.03-2.11 (m, 1H), 1.86-2.01 (m, 2H), 1.60 (t, J=18.69 Hz, 3H), 1.20-1.49 (m, 5H), 0.76 (t, J=7.33 Hz, 3H). MS(ES) [M+H]⁺ 480.3.

### Example 5

### (S)-5-(1-(1-(2,2-Difluoropropyl)piperidin-4-yl)propyl)-N-((4,6-dimethyl-2-oxo-1,2-dihydropyridin-3-yl)methyl)-4-methylthiophene-3-carboxamide

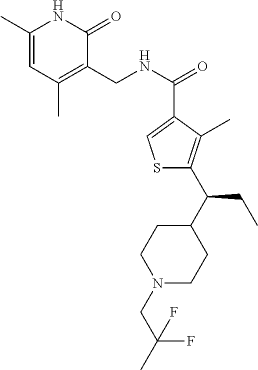 [SMILES: CC[C@H](c1scc(C(=O)NCc2c(C)cc(C)[nH]c2=O)c1C)C1CCN(CC(C)(F)F)CC1]

Following the procedure of Example 4f, (S)-5-(1-(1-(2,2-Difluoropropyl)piperidin-4-yl)propyl)-N-((4,6-dimethyl-2-oxo-1,2-dihydropyridin-3-yl)methyl)-4-methylthiophene-3-carboxamide was prepared. ¹H NMR (400 MHz, MeOH-d₄) δ 7.60 (s, 1H), 6.13 (s, 1H), 4.45 (s, 2H), 3.01 (d, J=11.62 Hz, 1H), 2.89 (d, J=10.61 Hz, 1H), 2.74 (ddd, J=3.79, 7.89, 11.05 Hz, 1H), 2.65 (t, J=14.15 Hz, 2H), 2.38 (s, 3H), 2.25 (d, J=6.06 Hz, 6H), 2.18 (dt, J=2.53, 11.62 Hz, 1H), 2.07 (dt, J=2.53, 11.62 Hz, 1H), 1.87-2.01 (m, 2H), 1.60 (t, J=18.82 Hz, 3H), 1.18-1.48 (m, 5H), 0.76 (t, J=7.20 Hz, 3H). MS(ES) [M+H]⁺ 480.3.

### Example 6

### N-((4,6-Dimethyl-2-oxo-1,2-dihydropyridin-3-yl)methyl)-5-(1-(1-(2-fluoro-2-methylpropyl)piperidin-4-yl)propyl)-4-methylthiophene-3-carboxamide

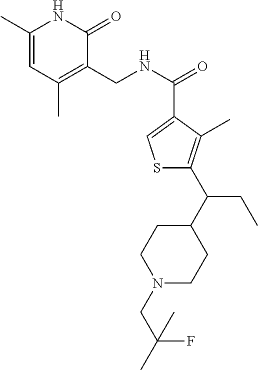 [SMILES: CCC(c1scc(C(=O)NCc2c(C)cc(C)[nH]c2=O)c1C)C1CCN(CC(C)(C)F)CC1]

Following the procedure of Example 4, racemic N-((4,6-dimethyl-2-oxo-1,2-dihydropyridin-3-yl)methyl)-5-(1-(1-(2-fluoro-2-methylpropyl)piperidin-4-yl)propyl)-4-methylthiophene-3-carboxamide was prepared. ¹H NMR (400 MHz, DMSO-d₆) δ 0.62-0.76 (m, 3H), 1.08-2.97 (m, 24H), 3.26-3.41 (m, 2H), 4.17-4.31 (m, 2H), 5.79-5.94 (m, 1H), 7.68 (s, 1H), 7.98 (t, J=4.93 Hz, 1H). MS(ES) [M+H]⁺ 476.3.

### Example 7

### N-((4,6-Dimethyl-2-oxo-1,2-dihydropyridin-3-yl)methyl)-5-(1-(1-(dimethylamino)piperidin-4-ylidene)propyl)-4-methylthiophene-3-carboxamide

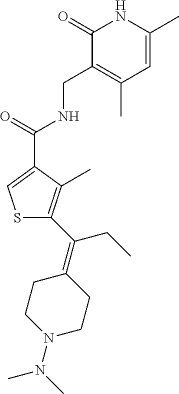 [SMILES: CCC(=C1CCN(N(C)C)CC1)c1scc(C(=O)NCc2c(C)cc(C)[nH]c2=O)c1C]

### a) Methyl 5-(1-(1-(dimethylamino)piperidin-4-ylidene)propyl)-4-methylthiophene-3-carboxylate

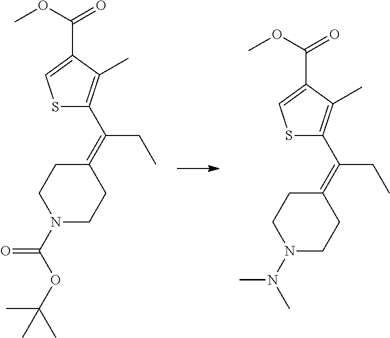 [SMILES: CCC(=C1CCN(C(=O)OC(C)(C)C)CC1)c1scc(C(=O)OC)c1C ; CCC(=C1CCN(N(C)C)CC1)c1scc(C(=O)OC)c1C]

To tert-butyl 4-(1-(4-(methoxycarbonyl)-3-methylthiophen-2-yl)propylidene)piperidine-1-carboxylate (1.0 g, 2.63 mmol) was added HCl in dioxane (20 mL, 658 mmol). After stirring for 30 min the reaction was evaporated to dryness under vacuum to give the de-protected piperidine HCl salt as a white solid foam.

To a solution of the white solid in AcOH (10 mL) was added a solution of NaNO₂ (0.46 g, 6.67 mmol) in water (2.5 mL) dropwise in portions over 2 h. The reaction was monitored by LCMS. After 2 h the reaction was 91% complete (N-nitroso intermediate MS(ES) [M+H]′ 309.2). To the reaction was slowly added in portions zinc powder (1.5 g, 22.94 mmol). The reaction became warm to the touch and was cooled in an ice bath. After stirring for 2 h at RT the reaction was filtered through a pad of Celite® to remove the zinc and rinsed with a small volume of MeOH (15 mL). To the filtrate was added 37 wt % formaldehyde in water (2.0 mL, 26.9 mmol). To the stirred mixture was added NaBH(OAc)₃ (2.3 g, 10.85 mmol) in potions over 30 min. The reaction was stirred overnight at RT. LCMS showed 17% desired dimethyl hydrazine. The reaction mixture was evaporated to dryness under vacuum, taken up in DCM, treated with 1 N Na₂CO₃ and stirred for 30 min. The suspension was filtered through a pad of Celite® and rinsed with a small volume of DCM. The clear filtrate was transferred to a reparatory funnel. The lower organic phase containing the product was removed, dried (Na₂SO₄), filtered, and concentrated under vacuum. The residue was purified by silica gel chromatography (Isco RediSep® Rf Gold 40 g, 0 to 8% EtOH in EtOAc). (The product eluted off the column at 3 to 5% EtOH.) The fractions containing product were combined and evaporated to dryness to give methyl 5-(1-(1-(dimethylamino)piperidin-4-ylidene)propyl)-4-methylthiophene-3-carboxylate (200 mg, 0.360 mmol, 13.65% yield) as a light yellow oil. MS(ES) [M+H]⁺ 323.2.

### b) N-((4,6-Dimethyl-2-oxo-1,2-dihydropyridin-3-yl)methyl)-5-(1-(1-(dimethylamino)piperidin-4-ylidene)propyl)-4-methylthiophene-3-carboxamide

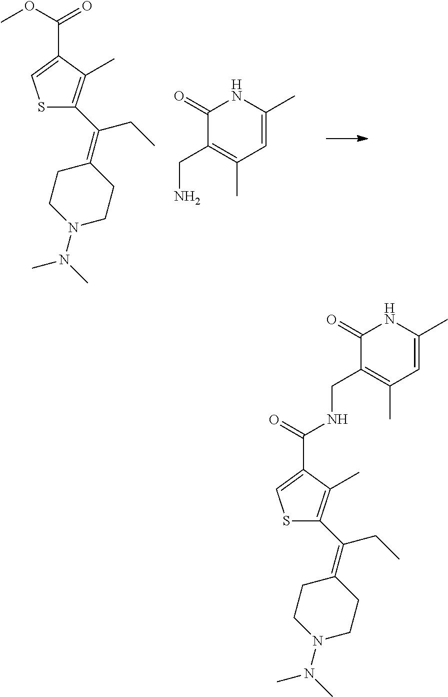

To a solution of methyl 5-(1-(1-(dimethylamino)piperidin-4-ylidene)propyl)-4-methylthiophene-3-carboxylate (200 mg, 0.620 mmol) in MeOH (20 mL) was added 1 N NaOH (4 mL, 4.00 mmol). The reaction was heated at 70° C. for 4 h. The reaction was concentrated under vacuum to remove the MeOH and neutralized with 1 N HCl (4.0 mL). A white solid suspension formed. The mixture was evaporated under vacuum to give the crude carboxylic acid as a white solid.

To the above was added 3-(aminomethyl)-4,6-dimethylpyridin-2(1H)-one hydrochloride (150 mg, 0.795 mmol), HOAt (84 mg, 0.62 mmol), DCM (20 mL) and NMM (90 μL, 0.819 mmol). The solid clumps were broken up with a stir rod. To the stirring suspension was added EDC free base (130 mg, 0.837 mmol). The reaction was stirred at RT for 2 h, then for 4 h at 40° C. with a reflux condensor attached. The cloudy solution was filtered through a pad of Celite® and rinsed with a small volume of DCM. The clear filtrate was concentrated and purified by silica gel chromatography (Isco RediSep® Rf Gold 40 g, 2 to 10% (5% NH₄OH/MeOH) in DCM). The pure fractions were combined and evaporated to dryness under vacuum. The residue was repurified by silca gel chromatography (Isco RediSep® Rf Gold 40 g, 15 to 40% EtOH in EtOAc). The pure fractions were combined, concentrated under vacuum, triturated with hexanes, filtered, washed with hexanes and dried under vacuum to give N-((4,6-dimethyl-2-oxo-1,2-dihydropyridin-3-yl)methyl)-5-(1-(1-(dimethylamino)piperidin-4-ylidene)propyl)-4-methylthiophene-3-carboxamide (144 mg, 0.325 mmol, 52.5% yield) as a white solid. ¹H NMR (400 MHz, DMSO-d₆) δ 11.49 (s, 1H), 8.02 (t, J=5.1 Hz, 1H), 7.78 (s, 1H), 5.86 (s, 1H), 4.24 (d, J=5.1 Hz, 2H), 2.61 (br. s., 2H), 2.45 (br. s., 2H), 2.40 (t, J=5.4 Hz, 3H), 2.26 (br. s., 2H) 2.25 (s, 6H), 2.18 (s, 3H), 2.11 (s, 3H), 2.06 (s, 3H), 1.94 (t, J=5.4 Hz, 2H), 0.86 (t, J=7.5 Hz, 3H). MS(ES) [M+H]⁺ 443.3.

### Example 8

### N-((4,6-Dimethyl-2-oxo-1,2-dihydropyridin-3-yl)methyl)-5-(1-(1-hydroxypiperidin-4-ylidene)propyl)-4-methylthiophene-3-carboxamide

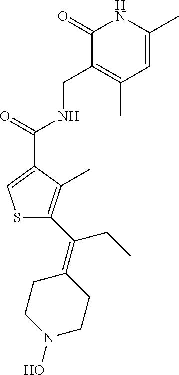 [SMILES: CCC(=C1CCN(O)CC1)c1scc(C(=O)NCc2c(C)cc(C)[nH]c2=O)c1C]

### a) Methyl 5-(1-(1-(benzoyloxy)piperidin-4-ylidene)propyl)-4-methylthiophene-3-carboxylate

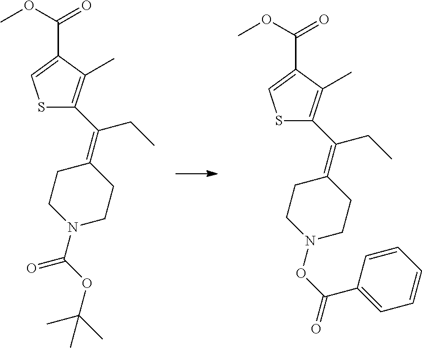 [SMILES: CCC(=C1CCN(C(=O)OC(C)(C)C)CC1)c1scc(C(=O)OC)c1C ; CCC(=C1CCN(OC(=O)c2ccccc2)CC1)c1scc(C(=O)OC)c1C]

To tert-butyl 4-(1-(4-(methoxycarbonyl)-3-methylthiophen-2-yl)propylidene)piperidine-1-carboxylate (1.0 g, 2.63 mmol) was added HCl in dioxane (20 mL, 658 mmol). The reaction was stirred for 30 min at RT, then evaporated to dryness under vacuum to give the amine hydrodrochloride as a white solid foam. To the above in THF (20 mL) was added Na₂HPO₄ (2.5 g, 17.61 mmol). The reaction was stirred and treated with benzoyl peroxide (1.0 g, 4.13 mmol) portionwise over 30 min. After stirring for 2 h at RT, no change was seen by LCMS. The reaction was stirred at 50° C. overnight. LCMS showed mostly desired product. The reaction was cooled to RT, evaporated to dryness under vacuum, taken up in EtOAc, washed with 1 N Na₂CO₃, brine, dried (Na₂SO4), filtered and evaporated to dryness. The residue was purified by silica gel chromatography (Isco RediSep® Rf Gold 80 g, 10 to 40% EtOAc in hexanes). The pure fractions were combined and evaporated to dryness under vacuum to give methyl 5-(1-(1-(benzoyloxy)piperidin-4-ylidene)propyl)-4-methylthiophene-3-carboxylate (0.64 g, 1.602 mmol, 60.8% yield) as a white foam. ¹H NMR (400 MHz, CDCl₃) δ 8.06 (s, 1H), 8.04-8.01 (m, 2H), 7.62-7.56 (m, 1H), 7.50-7.43 (m, 2H), 3.87 (s, 3H), 3.61 (br. s., 1H), 3.42 (br. s., 1H), 3.02 (br. s., 1H), 2.85 (br. s., 2H), 2.60 (br. s., 1H), 2.38 (br. s., 4H), 2.30 (br. s., 3H), 0.97 (t, J=7.5 Hz, 3H). MS(ES) [M+H]⁺ 400.2.

### b) N-((4,6-Dimethyl-2-oxo-1,2-dihydropyridin-3-yl)methyl)-5-(1-(1-hydroxypiperidin-4-ylidene)propyl)-4-methylthiophene-3-carboxamide

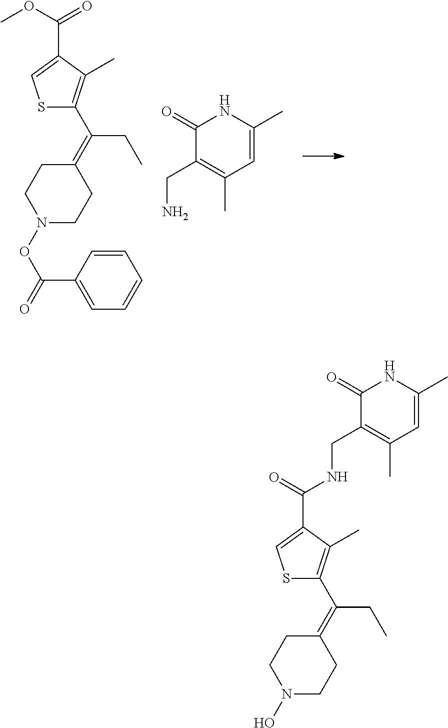

To a stirred solution of methyl 5-(1-(1-(benzoyloxy)piperidin-4-ylidene)propyl)-4-methylthiophene-3-carboxylate (0.64 g, 1.602 mmol) in MeOH (25 mL) was added 5 N NaOH (2.0 mL, 10.00 mmol). The reaction was heated at 70° C. for 16 h. The reaction was concentrated under vacuum to remove the MeOH, then neutralized with 6 N HCl (1.7 mL). A white solid suspension formed. The mixture was evaporated under vacuum to give the crude de-benzoylated carboxylic acid as an off-white solid, contaminated with benzoic acid.

To the above was added 3-(aminomethyl)-4,6-dimethylpyridin-2(1H)-one hydrochloride (0.604 g, 3.20 mmol), DCM (40 mL) and NMM (0.352 mL, 3.20 mmol). The solid clumps were broken up with a stir rod. To the stirring suspension was added EDC free base (0.547 g, 3.52 mmol). The reaction was stirred at RT for 2 h, then heated at 40° C. for 4 h. LCMS showed the desired product as its O-benzoylated derivative (Note: the benzoic acid present in the reaction mixture led to re-benzoylation) and other impurities. The cloudy solution was filtered through a pad of Celite® and rinsed with a small volume of DCM. The clear filtrate was concentrated and purified by silica gel chromatography (Isco RediSep® Rf Gold 40 g, 0 to 10% EtOH in EtOAc). Fractions containing the O-benzoylated product were combined and evaporated to dryness. The residue was taken up in MeOH (25 mL) and treated with 5 N NaOH (1.5 mL). The reaction was heated at 70° C. overnight. The reaction was neutralized with 6 N HCl (1.3 mL) and evaporated to dryness under vacuum. The residue was taken up in DCM, washed with aqueous NaHCO₃, dried (Na₂SO₄), filtered, and concentrated under vacuum. Purification of the residue by silica gel chromatography (Isco RediSep® Rf Gold 40 g, 10 to 20% EtOH in EtOAc) gave N-((4,6-dimethyl-2-oxo-1,2-dihydropyridin-3-yl)methyl)-5-(1-(1-hydroxypiperidin-4-ylidene)propyl)-4-methylthiophene-3-carboxamide (251 mg, 0.604 mmol, 37.7% yield) as a white solid. ¹H NMR (400 MHz, DMSO-d₆) δ 11.49 (br. s., 1H), 8.02 (t, J=5.1 Hz, 1H), 7.97 (s, 1H), 7.79 (s, 1H), 5.87 (s, 1H), 4.24 (d, J=4.8 Hz, 2H), 3.16 (br. s., 1H), 2.98 (br. s., 1H), 2.73-2.66 (m, 1H), 2.42-2.32 (m, 1H), 2.31-2.17 (m, 2H), 2.18 (s, 3H), 2.11 (s, 3H), 2.07 (br. s., 3H), 2.02 (br. s., 1H), 1.92 (br. s., 1H), 0.86 (t, J=7.5 Hz, 3H). MS(ES) [M+H]⁺ 416.2.

### Example 9

### 5-(4,6-Dimethyl-2-oxo-1,2-dihydropyridin-3-yl)methyl)-2-(1-(4-(dimethylamino)piperidin-1-yl)propyl)-3-methyl-6,7-dihydrothieno[3,2-c]pyridin-4(5H)-one

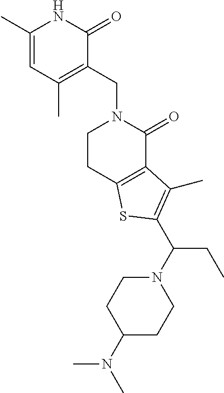 [SMILES: CCC(c1sc2c(c1C)C(=O)N(Cc1c(C)cc(C)[nH]c1=O)CC2)N1CCC(N(C)C)CC1]

### a) (Z)-4-Methyl-2-(2-nitrovinyl)thiophene

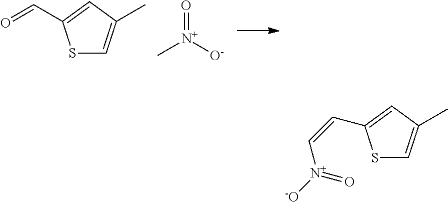

A solution of 4-methylthiophene-2-carbaldehyde (10.0 g, 79.3 mmol), CH₃NO₂ (100 mL) and NH₄OAc (1.1 g, 14.27 mmol) was heated at 100° C. for 4 h. The reaction was allowed to cool to RT and concentrated uner vacuum. The residue was taken up in EtOAc, washed with 1 N HCl, aq. NaHCO₃, brine, dried (MgSO₄), filtered, and evaporated to dryness. The residue was purified by silica gel chromatography (Isco RediSep® Rf Gold 120 g, 0 to 15% EtOAc in hexanes). The pure fractions were combined and evaporated to dryness under vacuum to give (Z)-4-methyl-2-(2-nitrovinyl)thiophene (9.63 g, 56.91 mmol, 71.8% yield) as a yellow oil (solidified under vacuum). ¹H NMR (400 MHz, CDCl₃) δ 8.10 (d, J=13.4 Hz, 1H), 7.47 (d, J=13.4 Hz, 1H), 7.27 (s, 1H), 7.17 (s, 1H), 2.31 (d, J=0.8 Hz, 3H). MS(ES) [M+H]⁺ 170.0.

### b) Ethyl (2-(4-methylthiophen-2-yl)ethyl)carbamate

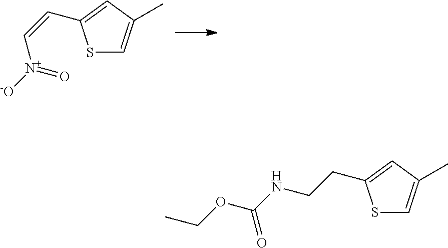

To a solution of 2 N LiBH₄ (120 mL, 240 mmol) under nitrogen was added dropwise TMSCl (60 mL, 473 mmol) over 10 min. The reaction became a white suspension. After stirring for 15 min a solution of (Z)-4-methyl-2-(2-nitrovinyl)thiophene (9.60 g, 56.74 mmol) in THF (50 mL) was added slowly dropwise over about 20 min. Vigorous gas evolution was seen. The reaction got slightly warm to the touch and was cooled in a water bath while periodically adding ice. The reaction was stirred at RT for 4 h, then warmed to 50° C. and stirred overnight. The reaction was cooled in an ice bath and carefully quenched with MeOH (200 mL). After stirring for 1 h the reaction was concentrated under vacuum to give crude 2-aminoethyl-4-methyl thiophene. MS(ES) [M+H]⁺ 142.1.

To a cooled (0° C.) solution of the crude 2-aminoethyl-4-methyl thiophene in DCM (200 mL) and water (100 mL) was slowly added Na₂CO₃ (25 g, 235.9 mmol) and EtOCOCl (0.710 mL, 7.39 mmol) dropwise. The resulting mixture was allowed to warm to RT and stirred for 1 h. The reaction was filtered through a pad of Celite® and the clear filtrate transferred to a separatory funnel. The lower organic phase was removed, dried (MgSO₄), filtered and concentrated under vacuum. The residue was purified by silica gel chromatography (Isco RediSep® Rf Gold 120 g, 10 to 30% EtOAc in hexanes). The pure fractions were combined and evaporated to dryness under vacuum to give ethyl (2-(4-methylthiophen-2-yl)ethyl)carbamate (9.29 g, 43.55 mmol, 76.7% yield) as a colorless oil. ¹H NMR (400 MHz, CDCl₃) δ 6.74 (s, 1H), 6.66 (s, 1H), 4.80 (br. s., 1H), 4.14 (q, J=6.9 Hz, 2H), 3.46 (q, J=6.3 Hz, 2H), 2.98 (t, J=6.6 Hz, 2H), 2.24 (d, J=0.8 Hz, 3H), 1.26 (t, J=7.1 Hz, 3H). MS(ES) [M+H]⁺ 214.1.

### c) 3-Methyl-6,7-dihydrothieno[3,2-c]pyridin-4(5H)-one

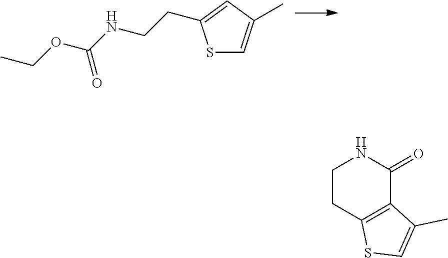

To ethyl (2-(4-methylthiophen-2-yl)ethyl)carbamate (9.20 g, 43.13 mmol) was added POCl₃ (100 mL, 107 mmol) and P₂O₅ (14 g, 98.6 mmol). The mixture was heated at reflux for 3 h (the mixture briefly formed a gummy ppt. which eventually dissolved with heating). The dark reaction mixture was allowed to cool to RT and evaporated to dryness under vacuum. The residue was carefully quenched with ice, basified with aq. Na₂CO₃, extracted with DCM, dried (Na₂SO₄), filtered, and concentrated under vacuum. The residue was purified by silica gel chromatography (Isco RediSep® Rf Gold 80 g, 30 to 80% EtOAc in hexanes). The pure fractions were combined and evaporated to dryness under vacuum, triturated with hexanes, filtered, and dried under vacuum to give 3-methyl-6,7-dihydrothieno[3,2-c]pyridin-4(5H)-one (2.22 g, 13.27 mmol, 30.78% yield) as an off-white solid. ¹H NMR (400 MHz, CDCl₃) δ 6.72 (m, 1H), 5.75 (br. s., 1H), 3.62 (t, J=6.2 Hz, 2H), 3.05 (t, J=6.7 Hz, 2H), 2.50 (d, J=1.3 Hz, 3H). MS(ES) [M+H]⁺ 168.0.

### d) 3-Methyl-2-propionyl-6,7-dihydrothieno[3,2-c]pyridin-4(5H)-one

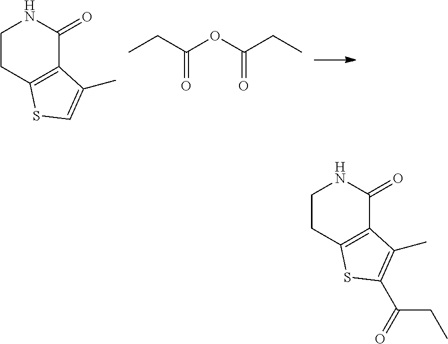

To a solution of 3-methyl-6,7-dihydrothieno[3,2-c]pyridin-4(5H)-one (1.6 g, 9.57 mmol) in CH₃NO₂ (50 mL) was added LiClO₄ (1.23 mg, 11.57 mmol), propionic anhydride (3.08 mL, 20.12 mmol) and In(OTf)₃ (308 mg, 0.547 mmol). The reaction was heated at 70° C. for 4 h. The reaction was allowed to cool to RT, diluted with water (200 mL), extracted with DCM (100 mL), dried (Na₂SO₄), filtered, and evaporated to dryness under vacuum. The residue was taken up in MeOH (200 mL). To the mixture was added K₂CO₃ (10.0 g, 72.4 mmol). The reaction was heated at 60° C. overnight. The reaction was evaporated to dryness, acidified with 1 N HCl (150 mL), extracted with DCM, dried (Na₂SO₄), filtered and concentrated under vacuum. The residue was purified by silica gel chromatography (Isco RediSep® Rf Gold 80 g, 20 to 50% EtOAc in DCM) (loaded in a large volume of DCM). The pure fractions were combined, evaporated to dryness, triturated with hexanes, filtered and dried under vacuum to give 3-methyl-2-propionyl-6,7-dihydrothieno[3,2-c]pyridin-4(5H)-one (210 1.59 g, 7.12 mmol, 74.4% yield) as a light yellow solid. ¹H NMR (400 MHz, CDCl₃) δ 5.97 (br. s., 1H), 3.63 (t, J=6.6 Hz, 2H), 3.08 (t, J=6.7 Hz, 2H), 2.88 (s, 3H), 2.87 (q, J=7.3 Hz, 2H), 1.23 (t, J=7.2 Hz, 3H). MS(ES) [M+H]⁺ 224.1.

### e) 2-(1-(4-(Dimethylamino)piperidin-1-yl)propyl)-3-methyl-6,7-dihydrothieno[3,2-c]pyridin-4(5H)-one

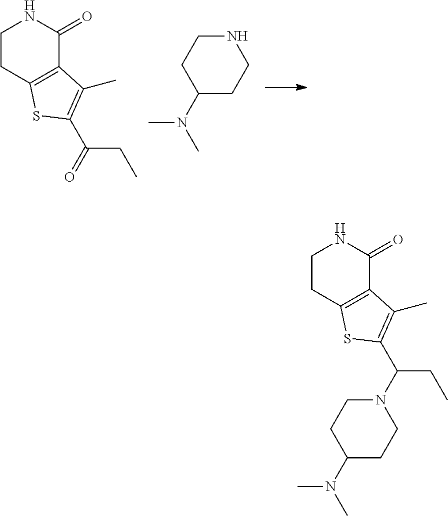

To 3-methyl-2-propionyl-6,7-dihydrothieno[3,2-c]pyridin-4(5H)-one (280 mg, 1.254 mmol) and N,N-dimethylpiperidin-4-amine (325 mg, 2.53 mmol) was added Ti(OiPr)₄ (0.80 mL, 2.73 mmol) and benzene (3 mL). The reaction was stirred at 80° C. for 18 h. The reaction was diluted with MeOH (3 mL), then NaBH₃CN (315 mg, 5.02 mmol) was added in two portions over 2 h. The reaction was heated at 60° C. and stirred for an additional 2 h. The reaction was evaporated to dryness under vacuum, taken up in (9:1) DCM/MeOH (15 mL) and treated with 1N Na₂CO₃ (10 mL). The resulting suspension was stirred for 30 min. The suspension was filtered through a pad of Celite® (slow) and rinsed with (9:1) DCM/MeOH (5 mL). The clear filtrate was transferred to a separatory funnel. The lower organic phase was removed, dried (Na₂SO₄), filtered and concentrated under vacuum. The residue was purified by silica gel chromatography (Isco RediSep® Rf Gold 40 g, 5 to 20% (5% NH₄OH/MeOH) in DCM). The pure fractions were combined and evaporated to dryness to give 2-(1-(4-(dimethylamino)piperidin-1-yl)propyl)-3-methyl-6,7-dihydrothieno[3,2-c]pyridin-4(5H)-one (360 mg, 1.019 mmol, 81% yield) as a colorless oil. (Solidified to a white solid foam under vacuum.). ¹H NMR (400 MHz, CDCl₃) δ 5.59 (br. s., 1H), 3.69 (dd, J=4.7, 9.5 Hz, 1H), 3.59 (dt, J=2.8, 6.8 Hz, 2H), 3.18-3.07 (m, 1H), 3.01 (t, J=6.8 Hz, 2H), 2.97-2.93 (m, 1H), 2.44 (s, 3H), 2.29 (s, 6H), 2.17-1.88 (m, 4H), 1.88-1.72 (m, 2H), 1.70-1.39 (m, 3H), 0.81 (t, J=7.3 Hz, 3H). MS(ES) [M+H]⁺ 336.3.

### f) 5-((2-(Benzyloxy)-4,6-dimethylpyridin-3-yl)methyl)-2-(1-(4-(dimethylamino)piperidin-1-yl)propyl)-3-methyl-6,7-dihydrothieno[3,2-c]pyridin-4(5H)-one

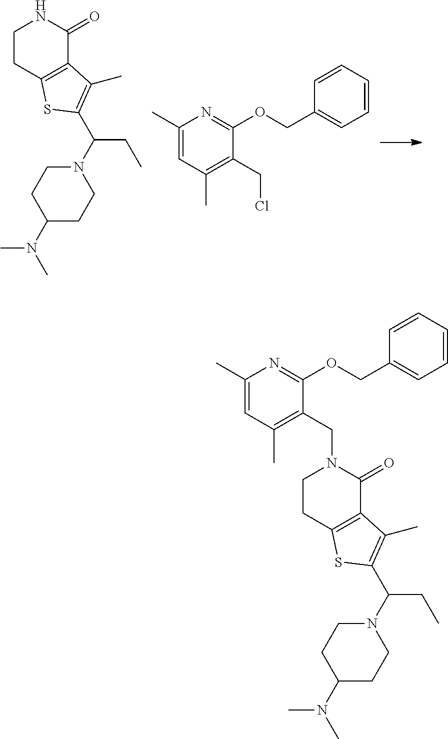

To a cooled (0° C.) solution of 2-(1-(4-(dimethylamino)piperidin-1-yl)propyl)-3-methyl-6,7-dihydrothieno[3,2-c]pyridin-4(5H)-one (350 mg, 1.043 mmol) in DMF (5 mL) was added dropwise 1 N KOtBu in THF (1.3 mL, 1.30 mmol) under an atmosphere of nitrogen. The mixture was stirred for 5 min, then 2-(benzyloxy)-3-(chloromethyl)-4,6-dimethylpyridine (350 mg, 1.337 mmol) in THF (1 mL) was added in one portion. After stirring for 15 min at 0° C., the mixture was quenched with saturated NH₄Cl (1.5 mL) and evaporated to near dryness under vacuum. The residue was diluted with aq. Na₂CO₃ (5 mL), extracted with DCM, dried (Na₂SO₄), filtered, and evaporated to dryness. The residue was purified by silica gel chromatography (Isco RediSep® Rf Gold 40 g, 0 to 15% (5% NH₄OH/MeOH) in DCM). The pure fractions were combined and evaporated to dryness under vacuum to give 5-((2-(benzyloxy)-4,6-dimethylpyridin-3-yl)methyl)-2-(1-(4-(dimethylamino)piperidin-1-yl)propyl)-3-methyl-6,7-dihydrothieno[3,2-c]pyridin-4(5H)-one (530 mg, 0.945 mmol, 91% yield) as a white solid. ¹H NMR (400 MHz, CDCl₃) δ 7.51-7.43 (m, 2H), 7.41-7.30 (m, 3H), 6.64 (s, 1H), 5.43 (s, 2H), 4.87-4.74 (m, 2H), 3.68 (dd, J=4.7, 9.5 Hz, 1H), 3.38 (t, J=6.8 Hz, 2H), 3.10 (d, J=10.9 Hz, 1H), 2.95 (dd, J=2.3, 11.1 Hz, 1H), 2.71 (t, J=6.8 Hz, 2H), 2.46 (s, 3H), 2.44 (s, 3H), 2.35 (s, 3H), 2.29 (s, 6H), 2.17-1.88 (m, 4H), 1.79 (t, J=15.3 Hz, 2H), 1.69-1.38 (m, 3H), 0.80 (t, J=7.3 Hz, 3H). MS(ES) [M+H]⁺ 561.4.

### g) 5-((4,6-Dimethyl-2-oxo-1,2-dihydropyridin-3-yl)methyl)-2-(1-(4-(dimethylamino)piperidin-1-yl)propyl)-3-methyl-6,7-dihydrothieno[3,2-c]pyridin-4(5H)-one

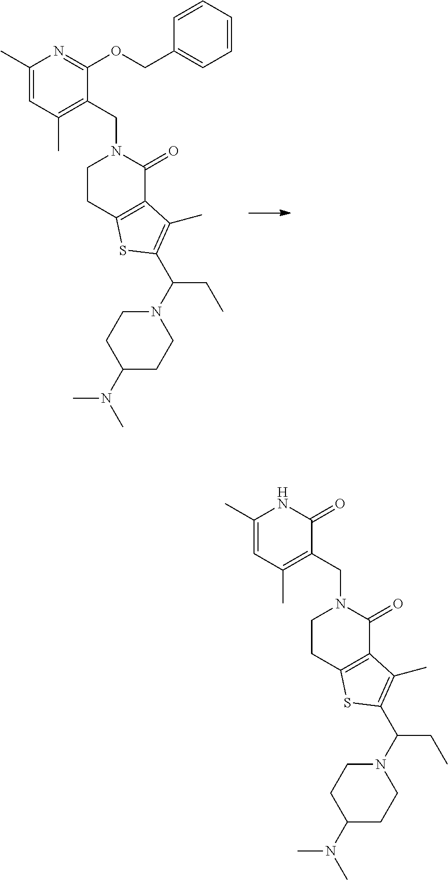 [SMILES: CCC(c1sc2c(c1C)C(=O)N(Cc1c(C)cc(C)nc1OCc1ccccc1)CC2)N1CCC(N(C)C)CC1 ; CCC(c1sc2c(c1C)C(=O)N(Cc1c(C)cc(C)[nH]c1=O)CC2)N1CCC(N(C)C)CC1]

To 5-((2-(benzyloxy)-4,6-dimethylpyridin-3-yl)methyl)-2-(1-(4-(dimethylamino)piperidin-1-yl)propyl)-3-methyl-6,7-dihydrothieno[3,2-c]pyridin-4(5H)-one (530 mg, 0.945 mmol) was added TFA (10 mL, 130 mmol). The solution was heated to 45° C. and stirred for 3 h. The reaction was evaporated to dryness under vacuum. The residue was basified with 1 N Na₂CO₃ (5 mL), extracted with DCM, dried (Na₂SO₄), filtered, and concentrated under vacuum. The residue was purified by silica gel chromatography (Isco RediSep® Rf Gold 40 g, 10 to 20% (5% NH₄OH/MeOH) in DCM). The pure fractions were combined, evaporated to dryness, triturated with hexanes, filtered and dried under vacuum to give 5-(4,6-dimethyl-2-oxo-1,2-dihydropyridin-3-yl)methyl)-2-(1-(4-(dimethylamino)piperidin-1-yl)propyl)-3-methyl-6,7-dihydrothieno[3,2-c]pyridin-4(5H)-one (520 mg, 1.050 mmol) as a white solid. ¹H NMR (400 MHz, DMSO-d₆) δ 11.54 (br. s., 1H), 5.88 (s, 1H), 4.54-4.42 (m, 2H), 3.71 (dd, J=5.2, 8.7 Hz, 1H), 3.50 (t, J=6.7 Hz, 2H), 3.07 (d, J=10.1 Hz, 1H), 2.89 (d, J=10.1 Hz, 1H), 2.85 (t, 3H), 2.68 (br. s., 1H), 2.51 (s, 6H) (buried under the DMSO peak), 2.36 (s, 3H), 2.16 (s, 3H), 2.12 (s, 3H), 2.04-1.76 (m, 5H), 1.59-1.34 (m, 3H), 0.75 (t, J=7.2 Hz, 3H). MS(ES) [M+H]⁺ 471.3.

### Example 10

### N-((4,6-Dimethyl-2-oxo-1,2-dihydropyridin-3-yl)methyl)-5-(1-(4-hydroxycyclohexylidene)propyl)-4-methylthiophene-3-carboxamide

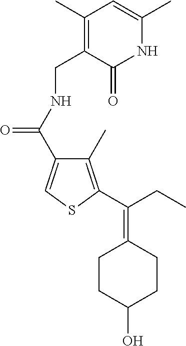 [SMILES: CCC(=C1CCC(O)CC1)c1scc(C(=O)NCc2c(C)cc(C)[nH]c2=O)c1C]

### a) Methyl 5-(1-(1,4-dioxaspiro[4.5]decan-8-ylidene)propyl)-4-methylthiophene-3-carboxylate

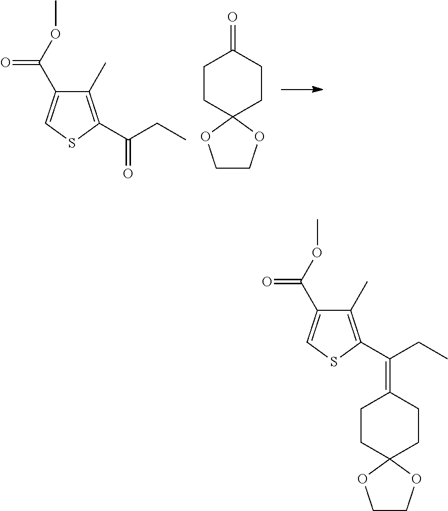

To a cooled (0° C.) suspension of zinc (12.60 g, 193 mmol) in THF (150 mL) was added dropwise TiCl₄ (10.26 mL, 93 mmol) through a short condensor. The mixture was heated at reflux for 2 h. The mixture was allowed to cool to rt and a solution of methyl 4-methyl-5-propionylthiophene-3-carboxylate (3 g, 14.13 mmol), 1,4-dioxaspiro[4.5]decan-8-one (6.62 g, 42.4 mmol) and pyridine (12.00 mL, 148 mmol) in THF (30 mL) was added. The reaction mixture was heated at reflux for 20 h. The mixture was allowed to cool to rt, treated with water (100 mL) and EtOAc (150 mL) and filtered through a short pad of Celite®. The blue solid was washed with EtOAc. The organic layer of filtrate was collected, dried (Na₂SO₄) and concentrated. The residue was purified using column chromatography (silica gel, 0 to 60% EtOAc/hexanes) to give methyl 5-(1-(1,4-dioxaspiro[4.5]decan-8-ylidene)propyl)-4-methylthiophene-3-carboxylate (1.71 g, 36%) as a colorless oil. MS(ES) [M+H]⁺ 337.2

### b) Methyl 4-methyl-5-(1-(4-oxocyclohexylidene)propyl)thiophene-3-carboxylate

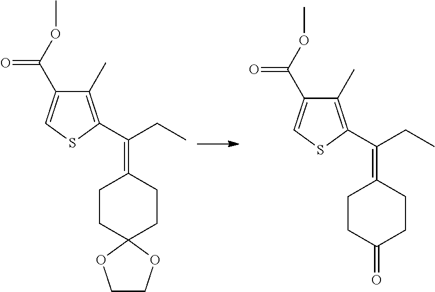 [SMILES: CCC(=C1CCC2(CC1)OCCO2)c1scc(C(=O)OC)c1C ; CCC(=C1CCC(=O)CC1)c1scc(C(=O)OC)c1C]

To a solution of methyl 5-(1-(1,4-dioxaspiro[4.5]decan-8-ylidene)propyl)-4-methylthiophene-3-carboxylate (1.5 g, 4.46 mmol) in 1,4-dioxane (20 mL) was added 6 N HCl (5.94 mL, 35.7 mmol) The mixture was stirred at rt for 18 h. The mixture was concentrated and the residue was treated with 10% NaHCO₃ and extracted with EtOAc (3×). The extract was dried (Na₂SO₄) and concentrated. The residue was purified using column chromatography (silica gel, 0 to 70% EtOAc/hexanes) to give methyl 4-methyl-5-(1-(4-oxocyclohexylidene)propyl)thiophene-3-carboxylate (1.1 g) as a colorless oil. MS(ES) [M+H]⁺ 293.2

### c) Methyl 5-(1-(4-hydroxycyclohexylidene)propyl)-4-methylthiophene-3-carboxylate

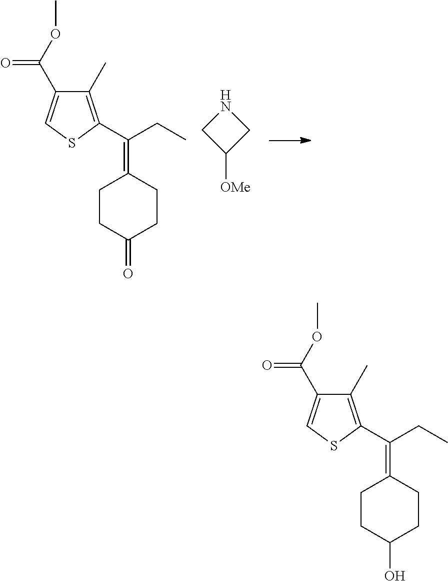

To a solution of methyl 4-methyl-5-(1-(4-oxocyclohexylidene)propyl)thiophene-3-carboxylate (110 mg, 0.376 mmol) in MeOH (2 mL) were added 3-methoxyazetidine hydrochloride (60.4 mg, 0.489 mmol), DIEA (0.085 mL, 0.489 mmol), and AcOH (0.043 mL, 0.752 mmol). The mixture was stirred at rt for 20 min, at which time NaBH₃CN (70.9 mg, 1.129 mmol) was added. The mixture was stirred at rt for 18 h. No reaction was detected by LCMS. The mixture was heated to 50° C. for 6 h. LCMS showed no desired product, but rather the reduced cyclohexanol. The mixture was quenched with 10% NaHCO₃ and extracted with EtOAc (3×). The extract was dried (Na₂SO₄) and concentrated. The residue was purified using column chromatography (silica gel, 0 to 60% EtOAc/hexanes) to give methyl 5-(1-(4-hydroxycyclohexylidene)propyl)-4-methylthiophene-3-carboxylate (36 mg) as an off-white solid. MS(ES) [M+H]⁺ 295.2

### d)N-((4,6-Dimethyl-2-oxo-1,2-dihydropyridin-3-yl)methyl)-5-(1-(4-hydroxycyclohexylidene)propyl)-4-methylthiophene-3-carboxamide

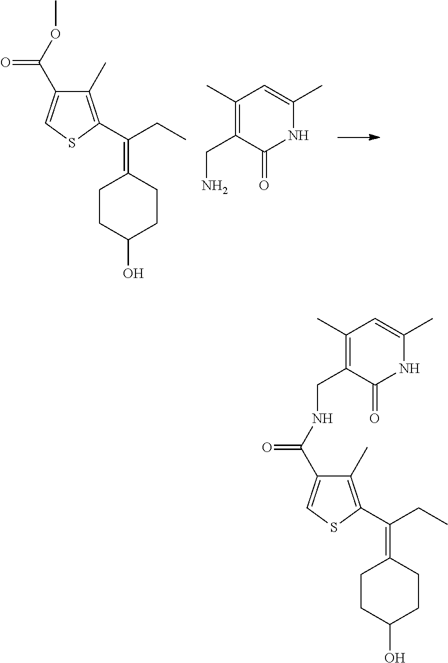

To a solution of methyl 5-(1-(4-hydroxycyclohexylidene)propyl)-4-methylthiophene-3-carboxylate (35 mg, 0.119 mmol) in MeOH (2 mL) was added NaOH (0.074 mL, 0.594 mmol). The mixture was heated at 40° C. for 18 h. The mixture was treated with HCl (0.099 mL, 0.594 mmol) and concentrated. The residue was dried under vacuum and treated with dimethyl sulfoxide (2.000 mL). To this mixture were added 3-(aminomethyl)-4,6-dimethylpyridin-2(1H)-one hydrochloride (29.2 mg, 0.155 mmol), NMM (0.078 mL, 0.713 mmol), EDC (45.6 mg, 0.238 mmol) and HOAt (32.4 mg, 0.238 mmol). The mixture was stirred at rt for 18 h. The reaction mixture was quenched with water (10 mL) and the resulting precipitate was collected by filtration and dried under vacuum to give N-((4,6-dimethyl-2-oxo-1,2-dihydropyridin-3-yl)methyl)-5-(1-(4-hydroxycyclohexylidene)propyl)-4-methylthiophene-3-carboxamide (45 mg, 88%) as an off-white solid. ¹H NMR (400 MHz, DMSO-d₆) δ 0.87 (t, J=7.45 Hz, 3H), 1.10-1.50 (m, 2H), 1.60-1.90 (m, 3H), 1.96-2.30 (m, 13H), 3.66 (m, 1H), 4.24 (d, J=5.05 Hz, 2H), 4.54 (d, J=4.04 Hz, 1H), 5.87 (s, 1H), 7.70-7.82 (m, 1H), 7.92-8.11 (m, 1H). MS(ES) [M+H]⁺ 415.2

### Example 11

### N-((4,6-Dimethyl-2-oxo-1,2-dihydropyridin-3-yl)methyl)-4-methyl-5-(1-(1-(2,2,2-trifluoroethyl)piperidin-4-yl)propyl)thiophene-3-carboxamide

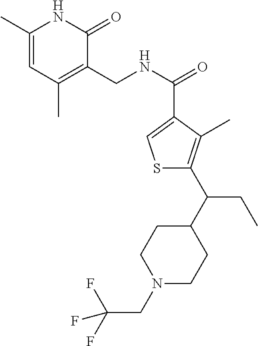 [SMILES: CCC(c1scc(C(=O)NCc2c(C)cc(C)[nH]c2=O)c1C)C1CCN(CC(F)(F)F)CC1]

Following the procedure of Example 4, racemic N-((4,6-Dimethyl-2-oxo-1,2-dihydropyridin-3-yl)methyl)-4-methyl-5-(1-(1-(2,2,2-trifluoroethyl)piperidin-4-yl)propyl)thiophene-3-carboxamide (117.7 mg, 0.243 mmol, 47.8% yield) was prepared as a white solid. ¹H NMR (400 MHz, DMSO-d₆) δ 11.47 (s, 1H), 7.98 (t, J=5.05 Hz, 1H), 7.69 (s, 1H), 5.86 (s, 1H), 4.23 (d, J=5.05 Hz, 2H), 3.07 (d, J=10.36 Hz, 2H), 2.90 (br. s., 1H), 2.83 (d, J=10.36 Hz, 1H), 2.64-2.77 (m, 1H), 2.26 (s, 1H), 2.15-2.20 (m, 6H), 2.11 (s, 3H), 1.82 (br. s., 2H), 1.03-1.42 (m, 6H), 0.69 (t, J=7.20 Hz, 3H). MS(ES) [M+H]⁺ 484.3.

### Example 12

### N-((4,6-Dimethyl-2-oxo-1,2-dihydropyridin-3-yl)methyl)-4-methyl-5-(1-(1-(oxazol-2-ylmethyl)piperidin-4-ylidene)propyl)thiophene-3-carboxamide

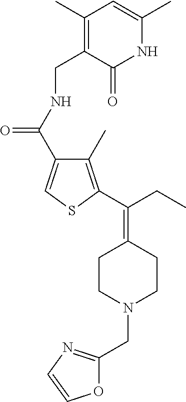 [SMILES: CCC(=C1CCN(Cc2ncco2)CC1)c1scc(C(=O)NCc2c(C)cc(C)[nH]c2=O)c1C]

### a) Methyl 4-methyl-5-(1-(1-(oxazol-2-ylmethyl)piperidin-4-ylidene)propyl)thiophene-3-carboxylate

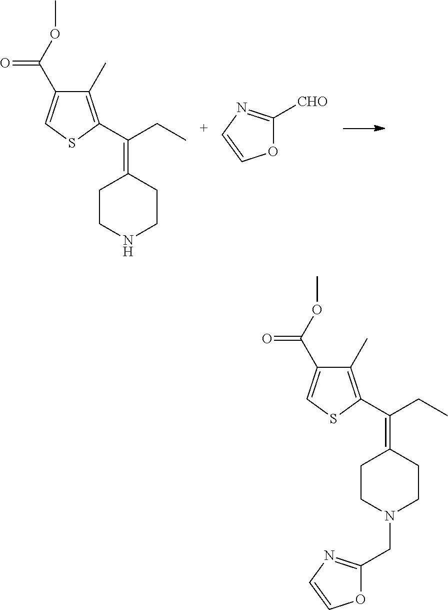

To a solution of methyl 4-methyl-5-(1-(piperidin-4-ylidene)propyl)thiophene-3-carboxylate hydrochloride (60 mg, 0.190 mmol) in MeOH (2 mL) were added oxazole-2-carbaldehyde (23.97 mg, 0.247 mmol), DIEA (0.043 mL, 0.247 mmol), and AcOH (0.023 mL, 0.399 mmol). The mixture was stirred for 20 min, at which time NaBH₃CN (47.7 mg, 0.760 mmol) was added. The mixture was stirred at RT for 18 h. The mixture was quenched with 10% NaHCO₃ and extracted with EtOAc (3×). The extract was dried (Na₂SO₄) and concentrated. The residue was purified using column chromatography (silica gel, 0 to 80% EtOAc/hexanes) to give methyl 4-methyl-5-(1-(1-(oxazol-2-ylmethyl)piperidin-4-ylidene)propyl)thiophene-3-carboxylate (49 mg) as a colorless oil. MS(ES) [M+H]⁺ 361.2.

### b) N-((4,6-Dimethyl-2-oxo-1,2-dihydropyridin-3-yl)methyl)-4-methyl-5-(1-(1-(oxazol-2-ylmethyl)piperidin-4-ylidene)propyl)thiophene-3-carboxamide

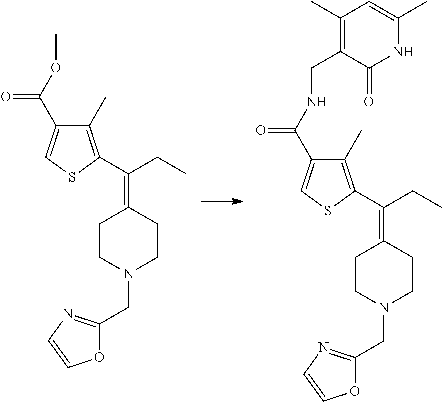 [SMILES: CCC(=C1CCN(Cc2ncco2)CC1)c1scc(C(=O)OC)c1C ; CCC(=C1CCN(Cc2ncco2)CC1)c1scc(C(=O)NCc2c(C)cc(C)[nH]c2=O)c1C]

Following the general procedure of Example 1c, N-((4,6-dimethyl-2-oxo-1,2-dihydropyridin-3-yl)methyl)-4-methyl-5-(1-(1-(oxazol-2-ylmethyl)piperidin-4-ylidene)propyl)thiophene-3-carboxamide (27 mg, 42% yield) was prepared. ¹H NMR (400 MHz, DMSO-d₆) δ 0.85 (t, J=7.45 Hz, 3H), 1.71-1.80 (m, 1H), 1.93 (t, J=5.43 Hz, 2H), 2.05 (s, 3H), 2.11 (s, 3H), 2.18 (s, 3H), 2.19-2.45 (m, 5H), 3.52-3.75 (m, 3H), 4.23 (d, J=5.05 Hz, 2H), 5.86 (s, 1H), 7.16 (d, J=0.76 Hz, 1H), 7.78 (s, 1H), 7.93-8.15 (m, 2H). MS(ES) [M+H]′ 481.3.

### Example 13

### N-((4,6-Dimethyl-2-oxo-1,2-dihydropyridin-3-yl)methyl)-4-methyl-5-(1-(1-(pyrimidin-2-yl)piperidin-4-ylidene)propyl)thiophene-3-carboxamide

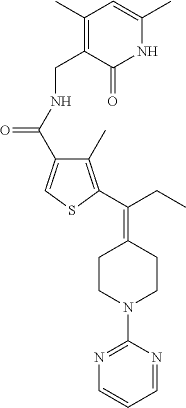 [SMILES: CCC(=C1CCN(c2ncccn2)CC1)c1scc(C(=O)NCc2c(C)cc(C)[nH]c2=O)c1C]

### a) Methyl 4-methyl-5-(1-(1-(pyrimidin-2-yl)piperidin-4-ylidene)propyl)thiophene-3-carboxylate

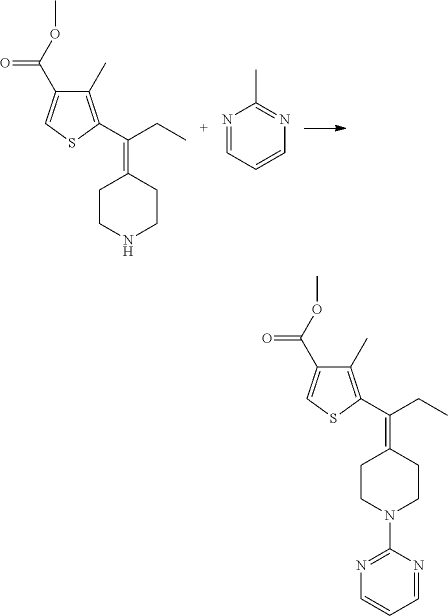

To a solution of methyl 4-methyl-5-(1-(piperidin-4-ylidene)propyl)thiophene-3-carboxylate hydrochloride (98 mg, 0.310 mmol) in 1,4-dioxane (3 mL) were added 2-chloropyrimidine (42.6 mg, 0.372 mmol) and K₂CO₃ (51.5 mg, 0.372 mmol). The mixture was heated at reflux for 18 h. The mixture was filtered and concentrated. The residue was purified using column chromatography (silica gel, 0 to 100% EtOAc/hexanes) to give methyl 4-methyl-5-(1-(1-(pyrimidin-2-yl)piperidin-4-ylidene)propyl)thiophene-3-carboxylate (84 mg) as an off-white solid. MS(ES) [M+H]⁺ 358.2.

### b) N-((4,6-Dimethyl-2-oxo-1,2-dihydropyridin-3-yl)methyl)-4-methyl-5-(1-(1-(pyrimidin-2-yl)piperidin-4-ylidene)propyl)thiophene-3-carboxamide

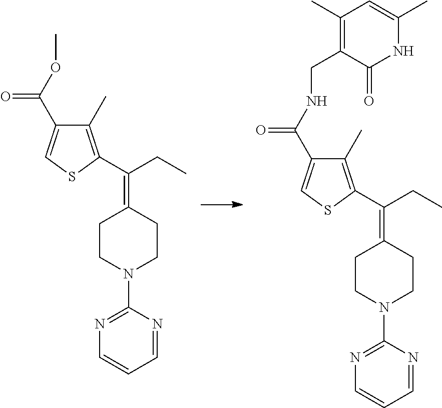 [SMILES: CCC(=C1CCN(c2ncccn2)CC1)c1scc(C(=O)OC)c1C ; CCC(=C1CCN(c2ncccn2)CC1)c1scc(C(=O)NCc2c(C)cc(C)[nH]c2=O)c1C]

Following the general procedure of Example 1c, N-((4,6-dimethyl-2-oxo-1,2-dihydropyridin-3-yl)methyl)-4-methyl-5-(1-(1-(pyrimidin-2-yl)piperidin-4-ylidene)propyl)thiophene-3-carboxamide (91 mg, 80% yield) was prepared. ¹H NMR (400 MHz, DMSO-d₆) δ 0.85 (t, J=7.45 Hz, 3H), 1.71-1.80 (m, 1H), 1.93 (t, J=5.43 Hz, 2H), 2.05 (s, 3H), 2.11 (s, 3H), 2.18 (s, 3H), 2.19-2.45 (m, 5H), 3.52-3.75 (m, 3H), 4.23 (d, J=5.05 Hz, 2H), 5.86 (s, 1H), 7.16 (d, J=0.76 Hz, 1H), 7.78 (s, 1H), 7.93-8.15 (m, 2H). MS(ES) [M+H]⁺ 478.3.

### Example 14

### 5-(1-(1-(2,2-Difluoroethyl)piperidin-4-yl)propyl)-N-((4,6-dimethyl-2-oxo-1,2-dihydropyridin-3-yl)methyl)-4-methylthiophene-3-carboxamide

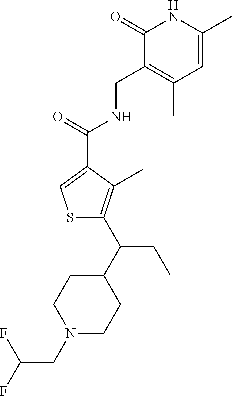 [SMILES: CCC(c1scc(C(=O)NCc2c(C)cc(C)[nH]c2=O)c1C)C1CCN(CC(F)F)CC1]

Following the procedure of Example 4, racemic 5-(1-(1-(2,2-difluoroethyl)piperidin-4-yl)propyl)-N-((4,6-dimethyl-2-oxo-1,2-dihydropyridin-3-yl)methyl)-4-methylthiophene-3-carboxamide (109 mg, 0.234 mmol) was prepared. ¹H NMR (400 MHz, CDCl₃) δ 12.78 (br. s., 1H), 7.43 (s, 1H), 7.38 (t, J=5.56 Hz, 1H), 5.94-6.02 (m, 1H), 5.66-5.89 (m, 1H), 4.51 (d, J=5.81 Hz, 2H), 2.78-3.06 (m, 2H), 2.60-2.77 (m, 3H), 2.39 (s, 3H), 2.27 (s, 6H), 1.98-2.19 (m, 2H), 1.81-1.97 (m, 2H), 1.30-1.48 (m, 4H), 0.67-0.94 (m, 4H). MS(ES) [M+H]⁺ 466.2.

### Example 15

### 5-(1-(1-(N′-Cyano-N-methylcarbamimidoyl)piperidin-4-ylidene)propyl)-N-((4,6-dimethyl-2-oxo-1,2-dihydropyridin-3-yl)methyl)-4-methylthiophene-3-carboxamide

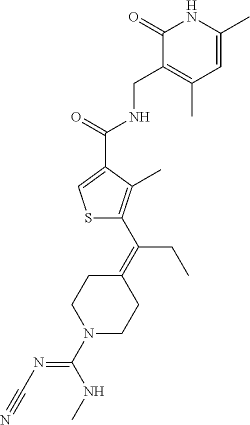 [SMILES: CCC(=C1CCN(/C(=N/C#N)NC)CC1)c1scc(C(=O)NCc2c(C)cc(C)[nH]c2=O)c1C]

### a) Methyl 5-(1-(1-(N′-cyano-N-methylcarbamimidoyl)piperidin-4-ylidene)propyl)-4-methylthiophene-3-carboxylate

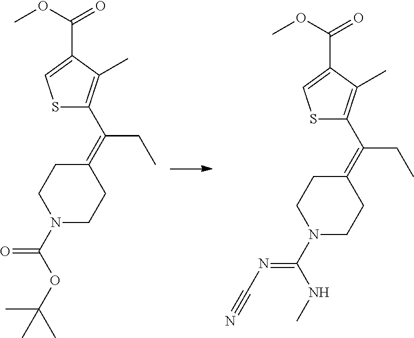 [SMILES: CCC(=C1CCN(C(=O)OC(C)(C)C)CC1)c1scc(C(=O)OC)c1C ; CCC(=C1CCN(/C(=N/C#N)NC)CC1)c1scc(C(=O)OC)c1C]

To tert-butyl 4-(1-(4-(methoxycarbonyl)-3-methylthiophen-2-yl)propylidene)piperidine-1-carboxylate (1.0 g, 2.63 mmol) was added HCl in 1,4-dioxane (15 mL, 60.0 mmol). The mixture was stirred at RT for 30 minutes then evaporated to dryness under vacuum to a solid foam.

To a solution of methyl isothiocyanate (200 mg, 2.74 mmol) in EtOH (10 mL) was added sodium hydrogencyanamide (180 mg, 2.81 mmol). The mixture was heated at reflux (80° C. oil bath) for 3 h, at which time it was allowed to cool to RT. To the mixture was added a solution of the above amine hydrochloride in DMF (5.0 mL). EDC free base (450 mg, 2.90 mmol) was added and the reaction was stirred for 2 h. The reaction mixture was evaporated under vacuum, taken up in DCM, washed with water, dried (Na₂SO₄), filtered and concentrated under vacuum. Purification of the residue by silica gel chromatography (Isco RediSep® Rf Gold 80 g, 0 to 10% EtOH in EtOAc) gave methyl 5-(1-(1-(N′-cyano-N-methylcarbamimidoyl)piperidin-4-ylidene)propyl)-4-methylthiophene-3-carboxylate (0.72 g, 1.997 mmol, 76% yield) as a white solid foam. ¹H NMR (400 MHz, CDCl₃) δ 8.05 (s, 1H), 5.06-4.99 (m, 1H), 3.87 (s, 3H), 3.62 (br. s., 2H), 3.38 (br. s., 2H), 3.05 (d, J=4.8 Hz, 3H), 2.59 (t, J=5.8 Hz, 2H), 2.39-2.30 (m, 2H), 2.24 (s, 3H), 2.16 (t, J=5.9 Hz, 2H), 0.94 (t, J=7.6 Hz, 3H). MS(ES) [M+H]⁺ 361.2.

### b) 5-(1-(1-(N′-Cyano-N-methylcarbamimidoyl)piperidin-4-ylidene)propyl)-N-((4,6-dimethyl-2-oxo-1,2-dihydropyridin-3-yl)methyl)-4-methylthiophene-3-carboxamide

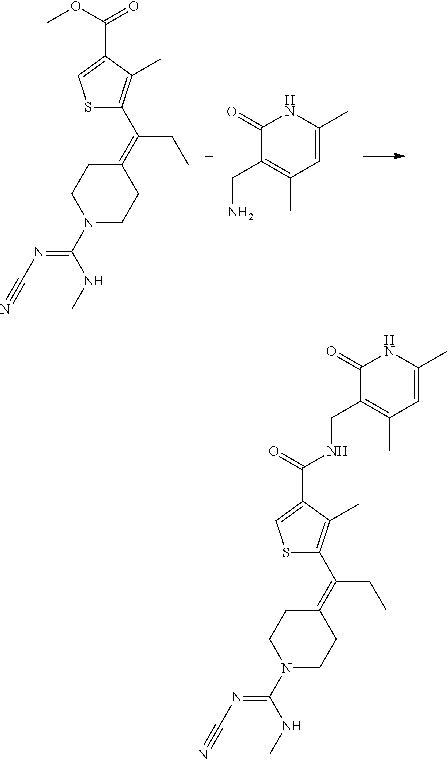

To a solution of methyl 5-(1-(1-(N′-cyano-N-methylcarbamimidoyl)piperidin-4-ylidene)propyl)-4-methylthiophene-3-carboxylate (0.70 g, 1.942 mmol) in MeOH (20 mL) was added 5 N NaOH (2.0 mL, 10.00 mmol). The reaction was stirred at 70° C. for 6 h. The reaction was concentrated under vacuum to remove the MeOH and neutralized with 6 N HCl (1.7 mL). A white gummy solid suspension formed. The mixture was evaporated under vacuum to give the crude carboxylic acid, contaminated with NaCl, as an off-white solid.

To the above was added 3-(aminomethyl)-4,6-dimethylpyridin-2(1H)-one hydrochloride (500 mg, 2.65 mmol), DCM (30 mL), HOAt (270 mg, 1.984 mmol) and NMM (0.30 mL, 2.73 mmol). The solid clumps were broken up with a stir rod. To the stirred suspension was added EDC free base (370 mg, 2.383 mmol). The reaction was stirred at RT for 2 h, then at 40° C. for 4 h. The cloudy mixture was filtered through a pad of Celite® and rinsed with a small volume of DCM. The clear filtrate was concentrated and purified by silica gel chromatography (Isco RediSep® Rf Gold 80 g, 10 to 25% EtOH in EtOAc). The pure fractions were combined, evaporated to dryness, triturated with EtOAc, hexanes, filtered, and dried under vacuum to give 5-(1-(1-(N′-cyano-N-methylcarbamimidoyl)piperidin-4-ylidene)propyl)-N-((4,6-dimethyl-2-oxo-1,2-dihydropyridin-3-yl)methyl)-4-methylthiophene-3-carboxamide (0.75 g, 1.56 mmol, 80% yield) as a white solid. ¹H NMR (400 MHz, DMSO-d₆) δ 11.48 (br. s., 1H), 8.03 (t, J=4.8 Hz, 1H), 7.81 (s, 1H), 7.20 (q, 1H), 5.87 (s, 1H), 4.24 (d, J=4.8 Hz, 2H), 3.51 (t, J=5.2 Hz, 2H), 3.32 (br. s., 2H), 2.84 (d, J=4.5 Hz, 3H), 2.47 (t, J=5.2 Hz, 2H), 2.33-2.23 (m, 2H), 2.18 (s, 3H), 2.11 (s, 3H), 2.07 (s, 3H), 2.02-1.97 (m, 2H), 0.87 (t, J=7.5 Hz, 3H). MS(ES) [M+H]⁺ 481.2.

### Example 16

### 2-(1-(1-(2,2-Difluoropropyl)piperidin-4-yl)propyl)-5-((4,6-dimethyl-2-oxo-1,2-dihydropyridin-3-yl)methyl)-3-methyl-6,7-dihydrothieno[3,2-c]pyridin-4(5H)-one

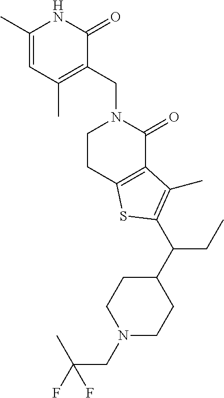 [SMILES: CCC(c1sc2c(c1C)C(=O)N(Cc1c(C)cc(C)[nH]c1=O)CC2)C1CCN(CC(C)(F)F)CC1]

### a) Methyl 4-methyl-5-(4,4,5,5-tetramethyl-1,3,2-dioxaborolan-2-yl)thiophene-3-carboxylate

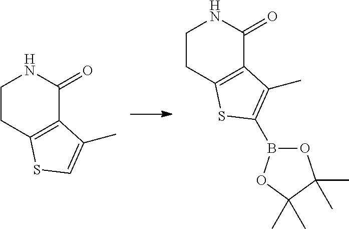 [SMILES: Cc1csc2c1C(=O)NCC2 ; Cc1c(B2OC(C)(C)C(C)(C)O2)sc2c1C(=O)NCC2]

To a 250 mL round bottom flask with a stir bar under nitrogen was added (1,5-cycloocatadiene)(methoxy)iridium(I) dimer (0.955 g, 1.440 mmol). With stirring, 4,4,5,5-tetramethyl-1,3,2-dioxaborolane (23.28 mL, 161 mmol) was added via syringe, followed by a solution of 4,4′-di-tert-butyl-2,2′-dipyridyl (0.773 g, 2.88 mmol) in n-hexane (150 mL). The dark mixture was stirred for 1 min, at which time methyl 4-methylthiophene-3-carboxylate (15 g, 96 mmol) was added dropwise (gas evolution). The reaction was stirred for 1.5 h. The reaction was evaporated to dryness under vacuum. The residue was purified by silica gel chromatography (Isco RediSep® Rf Gold 330 g, 0 to 100% DCM/hexanes, column pretreated with 3 column volumes of 1% Et₃N in chloroform) to give methyl 4-methyl-5-(4,4,5,5-tetramethyl-1,3,2-dioxaborolan-2-yl)thiophene-3-carboxylate (19.5 g, 65.7 mmol, 68.4% yield) as a light brown oil, which solidified upon standing. ¹H NMR (400 MHz, CDCl₃) δ 8.32 (s, 1H), 3.87 (s, 3H), 2.70 (s, 3H), 1.36 (s, 12H). MS(ES) [M+H]⁺ 283.2.

### b) (Z)-tert-Butyl 4-(1-(3-methyl-4-oxo-4,5,6,7-tetrahydrothieno[3,2-c]pyridin-2-yl)prop-1-en-1-yl)piperidine-1-carboxylate

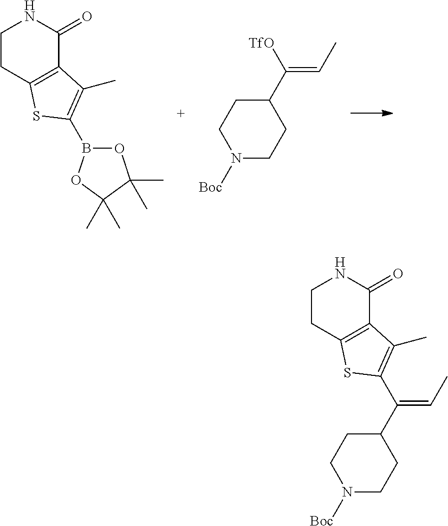 [SMILES: Cc1c(B2OC(C)(C)C(C)(C)O2)sc2c1C(=O)NCC2 ; C/C=C(\OS(=O)(=O)C(F)(F)F)C1CCN(C(=O)OC(C)(C)C)CC1 ; C/C=C(\c1sc2c(c1C)C(=O)NCC2)C1CCN(C(=O)OC(C)(C)C)CC1]

To a solution of 3-methyl-2-(4,4,5,5-tetramethyl-1,3,2-dioxaborolan-2-yl)-6,7-dihydrothieno[3,2-c]pyridin-4(5H)-one (0.82 g, 2.80 mmol) and (Z)-tert-butyl 4-(1-(((trifluoromethyl)sulfonyl)oxy)prop-1-en-1-yl)piperidine-1-carboxylate (1.044 g, 2.80 mmol) in 1,4-dioxane (30 mL) were added water (10 mL) and Na₂CO₃ (0.741 g, 6.99 mmol). The solution was degassed and Pd(PPh₃)₄ (0.162 g, 0.140 mmol) was added. The reaction mixture was heated at 70° C. for 1 h. The mixture was diluted with EtOAc (100 mL) and filtered. The layers were separated and the organics washed with brine, dried over MgSO₄, filtered and evaporated. The black residue was purified by column chromatography (50-100% EtOAc:hexanes) to give (Z)-tert-butyl 4-(1-(3-methyl-4-oxo-4,5,6,7-tetrahydrothieno[3,2-c]pyridin-2-yl)prop-1-en-1-yl)piperidine-1-carboxylate (950 mg, 2.433 mmol, 87% yield) as colorless oil. ¹H NMR (400 MHz, CDCl₃) δ 5.73-5.86 (m, 1H), 5.66 (br. s., 1H), 4.15 (br. s., 2H), 3.63 (t, J=6.69 Hz, 2H), 3.03 (t, J=6.69 Hz, 2H), 2.67 (t, J=13.01 Hz, 2H), 2.25 (s, 3H), 1.64-1.80 (m, 3H), 1.49-1.53 (m, 3H), 1.44-1.47 (m, 9H), 1.30-1.41 (m, 2H). MS(ES) [M+Na]⁺ 413.2.

### c) tert-Butyl 4-(1-(3-methyl-4-oxo-4,5,6,7-tetrahydrothieno[3,2-c]pyridin-2-yl)propyl)piperidine-1-carboxylate

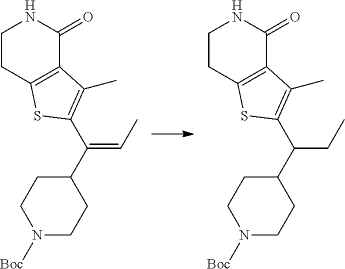 [SMILES: C/C=C(\c1sc2c(c1C)C(=O)NCC2)C1CCN(C(=O)OC(C)(C)C)CC1 ; CCC(c1sc2c(c1C)C(=O)NCC2)C1CCN(C(=O)OC(C)(C)C)CC1]

To a degassed (nitrogen) solution of (Z)-tert-butyl 4-(1-(3-methyl-4-oxo-4,5,6,7-tetrahydrothieno[3,2-c]pyridin-2-yl)prop-1-en-1-yl)piperidine-1-carboxylate (950 mg, 2.433 mmol) in EtOH (30 mL) was added 10% Pd/C (Degussa, 1.5 g, 1.410 mmol). The reaction mixture was stirred overnight under a hydrogen atmosphere (balloon). The reaction mixture was filtered and concentrated to give tert-butyl 4-(1-(3-methyl-4-oxo-4,5,6,7-tetrahydrothieno[3,2-c]pyridin-2-yl)propyl)piperidine-1-carboxylate (850 mg, 2.057 mmol, 85% yield) as a white solid. ¹H NMR (400 MHz, CDCl₃) δ 5.77 (br. s., 1H), 3.98-4.29 (m, 2H), 3.61 (t, J=6.82 Hz, 2H), 3.01 (t, J=6.69 Hz, 2H), 2.65-2.77 (m, 2H), 2.51-2.62 (m, 1H), 2.39 (s, 3H), 2.11 (d, J=12.88 Hz, 1H), 1.81-1.95 (m, 2H), 1.36-1.61 (m, 11H), 1.02-1.20 (m, 2H), 0.79 (t, J=7.33 Hz, 3H). MS(ES) [M+Na]⁺ 415.2

### d) tert-Butyl 4-(1-(5-((2-(benzyloxy)-4,6-dimethylpyridin-3-yl)methyl)-3-methyl-4-oxo-4,5,6,7-tetrahydrothieno[3,2-c]pyridin-2-yl)propyl)piperidine-1-carboxylate

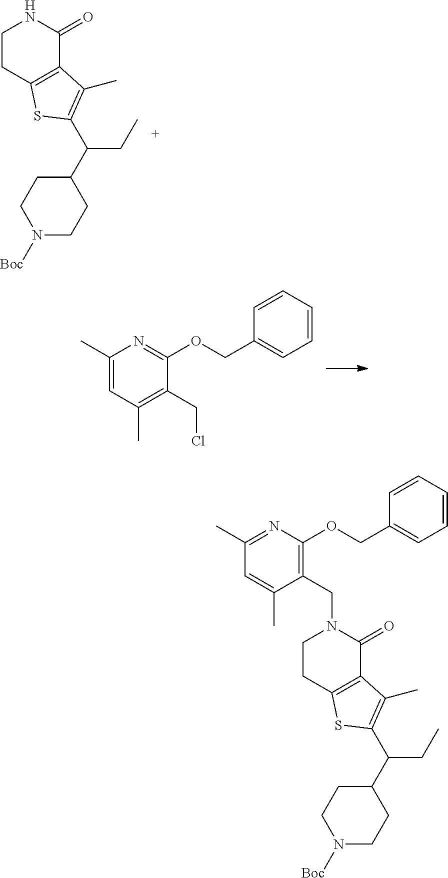 [SMILES: CCC(c1sc2c(c1C)C(=O)NCC2)C1CCN(C(=O)OC(C)(C)C)CC1 ; Cc1cc(C)c(CCl)c(OCc2ccccc2)n1 ; CCC(c1sc2c(c1C)C(=O)N(Cc1c(C)cc(C)nc1OCc1ccccc1)CC2)C1CCN(C(=O)OC(C)(C)C)CC1]

To a cooled (0° C. ice bath) solution of tert-butyl 4-(1-(3-methyl-4-oxo-4,5,6,7-tetrahydrothieno[3,2-c]pyridin-2-yl)propyl)piperidine-1-carboxylate (850 mg, 2.165 mmol) in DMF (10 mL) under an atmosphere of nitrogen was added dropwise 1 N KOtBu in THF (2.81 mL, 2.81 mmol). The mixture was stirred for 5 min, at which time a solution of 2-(benzyloxy)-3-(chloromethyl)-4,6-dimethylpyridine (737 mg, 2.81 mmol) in THF (5 mL) was added. The mixture was stirred at 0° C. for 15 min. The mixture was quenched with saturated NH₄Cl (5 mL). The mixture was diluted with water and EtOAc. The layers were separated and the organics were washed with water, brine, dried over MgSO₄, filtered, and evaporated to dryness. The residue was purified by silica gel chromatography (Isco RediSep® Rf Gold 40 g, 10 to 30% (EtOAc:hexanes) to give tert-butyl 4-(1-(5-((2-(benzyloxy)-4,6-dimethylpyridin-3-yl)methyl)-3-methyl-4-oxo-4,5,6,7-tetrahydrothieno[3,2-c]pyridin-2-yl)propyl)piperidine-1-carboxylate (1.2 g, 1.845 mmol, 85% yield) as a white solid. ¹H NMR (400 MHz, CDCl₃) δ 7.45-7.51 (m, 2H), 7.29-7.41 (m, 4H), 6.65 (s, 1H), 5.46 (s, 2H), 4.80 (s, 2H), 3.84-4.28 (m, 2H), 3.38 (t, J=6.69 Hz, 2H), 2.70 (t, J=6.69 Hz, 4H), 2.46 (s, 3H), 2.40 (s, 3H), 2.37 (s, 3H), 1.88 (dd, J=4.04, 7.33 Hz, 2H), 1.32-1.58 (m, 12H), 1.01-1.21 (m, 2H), 0.78 (t, J=7.33 Hz, 3H). MS(ES) [M+H]⁺ 618.4.

### e) 5-((2-(Benzyloxy)-4,6-dimethylpyridin-3-yl)methyl)-3-methyl-2-(1-(piperidin-4-yl)propyl)-6,7-dihydrothieno[3,2-c]pyridin-4(5H)-one

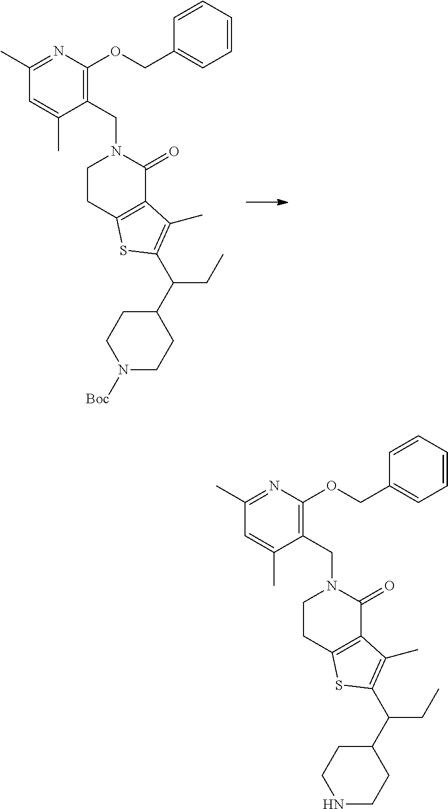 [SMILES: CCC(c1sc2c(c1C)C(=O)N(Cc1c(C)cc(C)nc1OCc1ccccc1)CC2)C1CCN(C(=O)OC(C)(C)C)CC1 ; CCC(c1sc2c(c1C)C(=O)N(Cc1c(C)cc(C)nc1OCc1ccccc1)CC2)C1CCNCC1]

To a cooled (10° C.) solution of tert-butyl 4-(1-(5-(2-(benzyloxy)-4,6-dimethylpyridin-3-yl)methyl)-3-methyl-4-oxo-4,5,6,7-tetrahydrothieno[3,2-c]pyridin-2-yl)propyl)piperidine-1-carboxylate (900 mg, 1.457 mmol) in 1,4-dioxane (10 mL) was added 4 M HCl in 1,4-dioxane (3 mL, 12.00 mmol). The reaction mixture was maintained at 10° C. for 1 h. The reaction was monitored by LCMS. After 1 h, additional 4 M HCl in 1,4-dioxane (3 mL, 12.00 mmol) was added. Upon consumption of starting material, the reaction mixture was neutralized with 6 M NaOH (pH-9) and extracted with DCM. The organic layer was washed with brine, dried over MgSO₄, filtered and evaporated. Purification of the residue by column chromatography (30% [5% NH₄OH/MeOH]: 70% DCM) gave 5-((2-(benzyloxy)-4,6-dimethylpyridin-3-yl)methyl)-3-methyl-2-(1-(piperidin-4-yl)propyl)-6,7-dihydrothieno[3,2-c]pyridin-4(5H)-one (450 mg, 0.826 mmol, 56.7% yield) as white solid. ¹H NMR (400 MHz, MeOH-d₄) δ 7.42 (dd, J=1.64, 7.96 Hz, 2H), 7.25-7.34 (m, 3H), 6.72 (s, 1H), 5.42 (s, 2H), 4.77 (s, 2H), 3.34-3.38 (m, 2H), 3.06 (d, J=12.38 Hz, 1H), 2.96 (d, J=12.38 Hz, 1H), 2.73-2.80 (m, 1H), 2.66-2.72 (m, 2H), 2.56 (dt, J=2.78, 12.38 Hz, 1H), 2.46 (dt, J=2.65, 12.44 Hz, 1H), 2.40 (s, 3H), 2.36 (s, 3H), 2.34 (s, 3H), 1.88-2.05 (m, 2H), 1.31-1.61 (m, 3H), 1.03-1.28 (m, 2H), 0.79 (t, J=7.33 Hz, 3H) MS(ES) [M+H]⁺ 518.3.

### f) 2-(1-(1-(2,2-Difluoropropyl)piperidin-4-yl)propyl)-5-((4,6-dimethyl-2-oxo-1,2-dihydropyridin-3-yl)methyl)-3-methyl-6,7-dihydrothieno[3,2-c]pyridin-4(5H)-one

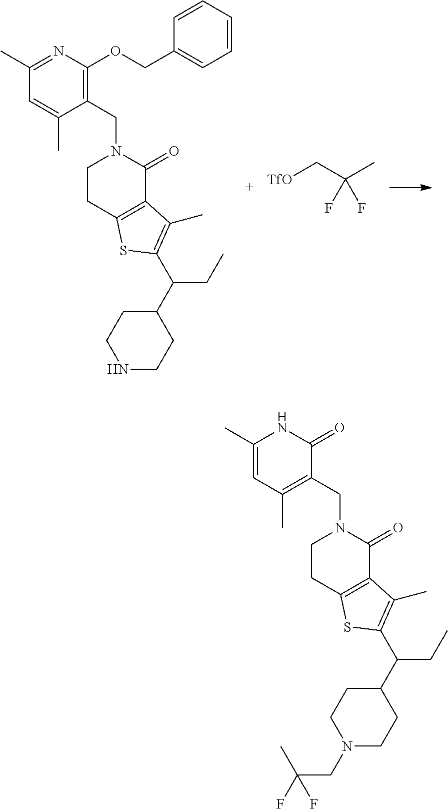 [SMILES: CCC(c1sc2c(c1C)C(=O)N(Cc1c(C)cc(C)nc1OCc1ccccc1)CC2)C1CCNCC1 ; CC(F)(F)COS(=O)(=O)C(F)(F)F ; CCC(c1sc2c(c1C)C(=O)N(Cc1c(C)cc(C)[nH]c1=O)CC2)C1CCN(CC(C)(F)F)CC1]

To a cooled (0° C.) solution of 2,2-difluoropropan-1-ol (193 mg, 2.010 mmol) and pyridine (0.163 mL, 2.010 mmol) in CH₃CN (20 mL) was added dropwise Tf₂O (0.312 mL, 1.849 mmol). The reaction was maintained at 0° C. for 30 min.

To a suspension of 5-((2-(benzyloxy)-4,6-dimethylpyridin-3-yl)methyl)-3-methyl-2-(1-(piperidin-4-yl)propyl)-6,7-dihydrothieno[3,2-c]pyridin-4(5H)-one (208 mg, 0.402 mmol) and K₂CO₃ (500 mg, 3.62 mmol) in CH₃CN (20 mL) was added the above cold reaction mixture (containing 2,2-difluoropropyl trifluoromethanesulfonate). The reaction was allowed to warm to RT, then heated to 50° C. overnight. The reaction was filtered and evaporated to dryness under vacuum. The residue was dissolved in EtOAc and the resultant solution was washed with water and brine, dried (MgSO₄), filtered, and concentrated under vacuum to give an oil.

A solution of the above oil in TFA (5 mL, 64.9 mmol) was maintained for 30 min, at which time it was concentrated. The reaction mixture was purified by preparative HPLC (5 to 60% MeCN:water with 0.1% formic acid). The product containing fractions were treated with 6 M HCl (0.5 mL) and concentrated to give 2-(1-(1-(2,2-difluoropropyl)piperidin-4-yl)propyl)-5-((4,6-dimethyl-2-oxo-1,2-dihydropyridin-3-yl)methyl)-3-methyl-6,7-dihydrothieno[3,2-c]pyridin-4(5H)-one (90 mg, 0.169 mmol, 42.1% yield) as white solid. ¹H NMR (400 MHz, MeOH-d₄) δ 7.07 (s, 1H), 4.82 (s, 2H), 3.88-3.99 (m, 2H), 3.72-3.86 (m, 3H), 3.65 (d, J=12.63 Hz, 1H), 3.07-3.27 (m, 4H), 2.80-2.91 (m, 1H), 2.69 (s, 3H), 2.54 (s, 3H), 2.40 (s, 2H), 2.27 (d, J=14.15 Hz, 1H), 2.00 (ddd, J=3.79, 7.20, 13.52 Hz, 1H), 1.57-1.89 (m, 7H), 1.47 (ddd, J=7.07, 11.18, 13.58 Hz, 1H), 0.80 (t, J=7.33 Hz, 3H). MS(ES) [M+H]⁺ 506.3.

### Example 17

### 5-((4,6-Dimethyl-2-oxo-1,2-dihydropyridin-3-yl)methyl)-2-(1-(1-(2-fluoropropyl)piperidin-4-yl)propyl)-3-methyl-6,7-dihydrothieno[3,2-c]pyridin-4(5H)-one

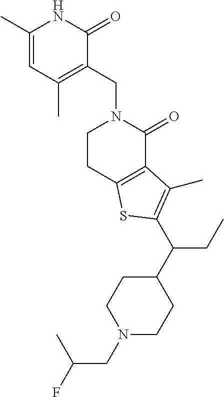 [SMILES: CCC(c1sc2c(c1C)C(=O)N(Cc1c(C)cc(C)[nH]c1=O)CC2)C1CCN(CC(C)F)CC1]

Following the procedure of Example 16, 5-((4,6-dimethyl-2-oxo-1,2-dihydropyridin-3-yl)methyl)-2-(1-(1-(2-fluoropropyl)piperidin-4-yl)propyl)-3-methyl-6,7-dihydrothieno[3,2-c]pyridin-4(5H)-one (110 mg, 0.199 mmol) was prepared. ¹H NMR (400 MHz, MeOH-d₄) δ 7.07 (s, 1H), 5.11-5.38 (m, 1H), 4.82 (s, 2H), 3.87-4.03 (m, 2H), 3.56-3.81 (m, 2H), 3.36-3.42 (m, 4H), 3.22-3.31 (m, 1H), 3.15 (t, J=6.95 Hz, 2H), 2.92-3.10 (m, 2H), 2.80-2.91 (m, 1H), 2.69 (s, 3H), 2.54 (s, 3H), 2.40 (s, 3H), 2.22-2.33 (m, 1H), 2.00 (ddd, J=3.79, 7.33, 13.64 Hz, 1H), 1.48-1.88 (m, 4H), 1.35-1.46 (m, 3H), 0.80 (t, J=7.20 Hz, 3H). MS(ES) [M+H]⁺ 488.3.

### Example 18

### N′-Cyano-4-(1-(5-((4,6-dimethyl-2-oxo-1,2-dihydropyridin-3-yl)methyl)-3-methyl-4-oxo-4,5,6,7-tetrahydrothieno[3,2-c]pyridin-2-yl)propyl)-N-methylpiperidine-1-carboximidamide

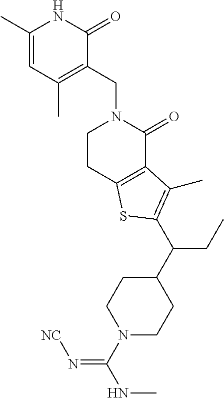 [SMILES: CCC(c1sc2c(c1C)C(=O)N(Cc1c(C)cc(C)[nH]c1=O)CC2)C1CCN(/C(=N\C#N)NC)CC1]

Following the procedure of Examples 15 and 16, N′-cyano-4-(1-(5-((4,6-dimethyl-2-oxo-1,2-dihydropyridin-3-yl)methyl)-3-methyl-4-oxo-4,5,6,7-tetrahydrothieno[3,2-c]pyridin-2-yl)propyl)-N-methylpiperidine-1-carboximidamide (35 mg, 0.065 mmol) was prepared. ¹H NMR (400 MHz, MeOH-d₄) δ 11.53 (s, 1H), 7.13 (d, J=4.8 Hz, 1H), 5.87 (s, 1H), 4.48 (s, 2H), 3.98 (d, J=12.6 Hz, 1H), 3.89 (d, J=12.9 Hz, 1H), 3.50 (t, J=6.7 Hz, 2H), 2.64-2.89 (m, 8H), 2.28-2.33 (m, 3H), 2.15 (s, 3H), 2.12 (s, 3H), 1.87 (d, J=11.4 Hz, 2H), 1.52-1.64 (m, 1H), 1.25-1.42 (m, 2H), 1.12-1.20 (m, 1H), 1.00-1.09 (m, 1H), 0.71 (t, J=7.2 Hz, 3H). MS(ES) [M+H]⁺ 509.0.

### Example 19

### 5-(1-(1-(N′-Cyano-N-methylcarbamimidoyl)piperidin-4-yl)propyl)-N-((4,6-dimethyl-2-oxo-1,2-dihydropyridin-3-yl)methyl)-4-methylthiophene-3-carboxamide

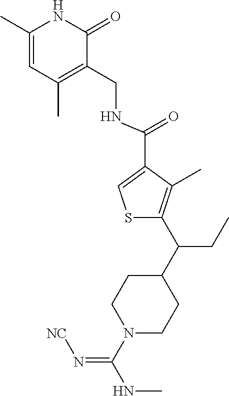 [SMILES: CCC(c1scc(C(=O)NCc2c(C)cc(C)[nH]c2=O)c1C)C1CCN(/C(=N\C#N)NC)CC1]

Following the procedure of Examples 4 and 15, 5-(1-(1-(N′-cyano-N-methylcarbamimidoyl)piperidin-4-yl)propyl)-N-((4,6-dimethyl-2-oxo-1,2-dihydropyridin-3-yl)methyl)-4-methylthiophene-3-carboxamide (49 mg, 0.096 mmol) was prepared. ¹H NMR (400 MHz, DMSO-d₆) δ 11.46 (br. s., 1H), 7.98 (t, J=4.9 Hz, 1H), 7.64-7.73 (m, 1H), 7.13 (q, J=4.3 Hz, 1H), 5.86 (s, 1H), 4.22 (d, J=5.1 Hz, 2H), 3.98 (d, J=13.4 Hz, 1H), 3.88 (d, J=13.1 Hz, 1H), 2.64-2.87 (m, 5H), 2.18 (d, J=2.5 Hz, 6H), 2.11 (s, 3H), 1.80-1.92 (m, 2H), 1.55-1.66 (m, 1H), 1.29-1.43 (m, 2H), 0.91-1.19 (m, 3H), 0.70 (t, J=7.3 Hz, 3H). MS(ES) [M+H]⁺ 483.

### Example 20

### 5-(1-(1-(2,2-Difluoroethyl)piperidin-4-ylidene)propyl)-N-((4,6-dimethyl-2-oxo-1,2-dihydropyridin-3-yl)methyl)-4-methylthiophene-3-carboxamide

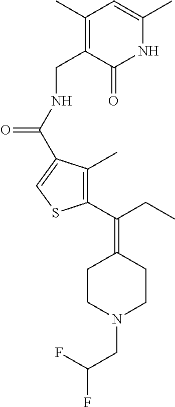 [SMILES: CCC(=C1CCN(CC(F)F)CC1)c1scc(C(=O)NCc2c(C)cc(C)[nH]c2=O)c1C]

Following the general procedure of Example 2, 5-(1-(1-(2,2-difluoroethyl)piperidin-4-ylidene)propyl)-N-((4,6-dimethyl-2-oxo-1,2-dihydropyridin-3-yl)methyl)-4-methylthiophene-3-carboxamide was prepared. ¹H NMR (400 MHz, DMSO-d₆) δ 11.48 (s, 1H), 8.02 (t, J=5.05 Hz, 1H), 7.79 (s, 1H), 5.95-6.32 (m, 1H), 5.86 (s, 1H), 4.23 (d, J=5.05 Hz, 2H), 2.72 (dt, J=4.29, 15.66 Hz, 2H), 2.60 (br. s., 2H), 2.44 (br. s., 2H), 2.35-2.41 (m, 2H), 2.21-2.31 (m, 2H), 2.18 (s, 3H), 2.11 (s, 3H), 2.07 (s, 3H), 1.93 (t, J=5.05 Hz, 2H), 0.86 (t, J=7.45 Hz, 3H). MS(ES) [M+H]⁺ 464.2.

### Example 21

### (R)-5-(1-(1-(2,2-Difluoroethyl)piperidin-4-yl)propyl)-N-((4,6-dimethyl-2-oxo-1,2-dihydropyridin-3-yl)methyl)-4-methylthiophene-3-carboxamide

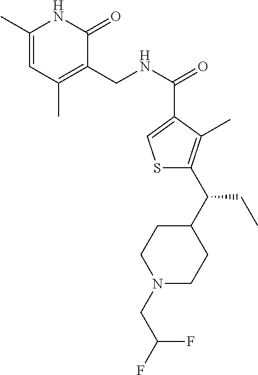 [SMILES: CC[C@@H](c1scc(C(=O)NCc2c(C)cc(C)[nH]c2=O)c1C)C1CCN(CC(F)F)CC1]

Following the general procedure of Example 4, (R)-5-(1-(1-(2,2-difluoroethyl)piperidin-4-yl)propyl)-N-((4,6-dimethyl-2-oxo-1,2-dihydropyridin-3-yl)methyl)-4-methylthiophene-3-carboxamide was prepared. ¹H NMR (400 MHz, DMSO-d₆) δ 0.69 (t, J=7.20 Hz, 3H) 1.09-1.23 (m, 2H) 1.25-1.39 (m, 3H) 1.75-1.90 (m, 2H) 1.93-2.02 (m, 1H) 2.02-2.09 (m, 1H) 2.11 (s, 3H) 2.18 (s, 6H) 2.56-2.74 (m, 3H) 2.80 (d, J=11.12 Hz, 1H) 2.89 (d, J=11.62 Hz, 1H) 4.22 (d, J=5.05 Hz, 2H) 5.86 (s, 1H) 6.06 (t, J=4.29 Hz, 1H) 7.68 (s, 1H) 7.98 (t, J=5.05 Hz, 1H) 11.47 (s, 1H). MS(ES) [M+H]⁺ 466.2.

### Example 22

### (R)-5-(1-(1-(2,2-Difluoroethyl)piperidin-4-yl)ethyl)-N-((4,6-dimethyl-2-oxo-1,2-dihydropyridin-3-yl)methyl)-4-methylthiophene-3-carboxamide

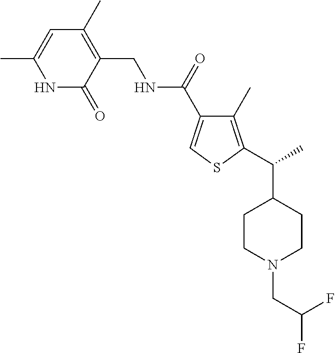 [SMILES: Cc1cc(C)c(CNC(=O)c2csc([C@H](C)C3CCN(CC(F)F)CC3)c2C)c(=O)[nH]1]

### a) Methyl 4-methyl-5-(4,4,5,5-tetramethyl-1,3,2-dioxaborolan-2-yl)thiophene-3-carboxylate

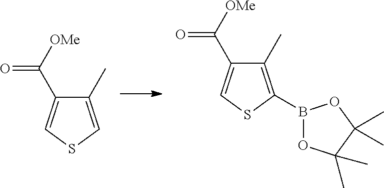

To a 100-mL round bottom flask charged with (1,5-cycooctadiene)(methoxy)iridinum(1)dimer (325 mg, 0.490 mmol) was added 4,4,5,5-tetramethyl-1,3,2-dioxaborolane (7.10 g, 55.5 mmol) with stirring, followed by a solution of 4,4′-di-tert-butyl-2,2′-dipyridine (260 mg, 0.969 mmol) in n-hexane (35 mL). The mixture was stirred at room temperature for 2 min and methyl 4-methylthiophene-3-carboxylate (5 g, 32 mmol) was added dropwise. The mixture was stirred at room temperature for 18 h. The reaction mixture was then concentrated and the residue was purified using column chromatography (silica gel, 0 to 100% DCM/hexanes) to give 5.8 g of product as a colorless oil. ¹H NMR (400 MHz, CDCl₃) δ 1.36 (s, 12H), 2.70 (s, 3H), 3.87 (s, 3H), 8.32 (s, 1H). MS(ES) [M+H]⁺ 283.1.

### b) tert-Butyl 4-(1-(4-(methoxycarbonyl)-3-methylthiophen-2-yl)vinyl)piperidine-1-carboxylate

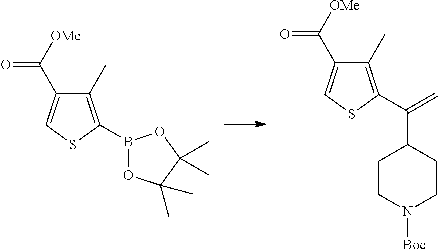 [SMILES: COC(=O)c1csc(B2OC(C)(C)C(C)(C)O2)c1C ; C=C(c1scc(C(=O)OC)c1C)C1CCN(C(=O)OC(C)(C)C)CC1]

To a solution of methyl 4-methyl-5-(4,4,5,5-tetramethyl-1,3,2-dioxaborolan-2-yl)thiophene-3-carboxylate (2.65 g, 9.39 mmol) in 1,4-dioxane (72 mL) were added tert-butyl 4-(1-(((trifluoromethyl)sulfonyl)oxy)vinyl)piperidine-1-carboxylate (3.38 g, 9.39 mmol), Na₂CO₃ (2.489 g, 23.48 mmol), and water (24 mL). The mixture was degassed for 10 min by bubbling N₂. Pd(PPh₃)₄ (0.543 g, 0.470 mmol) was added and the mixture was heated at 70° C. for 1 h. The reaction mixture was allowed to cool to RT and extracted with EtOAc (3×). The combined extracts were dried (Na₂SO₄) and concentrated. The residue was purified using column chromatography (silica gel, 0 to 40% EtOAc/hexanes) to give 2.8 g of product as a colorless oil. ¹H NMR (400 MHz, CDCl₃) δ 1.27-1.55 (m, 11H), 1.78 (m, 2H), 2.26-2.46 (m, 4H), 2.71 (t, J=11.49 Hz, 2H), 3.87 (s, 3H), 4.18 (br. s., 2H), 5.13 (s, 1H), 5.29-5.39 (m, 1H), 8.02 (s, 1H). MS(ES) [M+H]⁺ 388.1.

### c) (R)-tert-Butyl 4-(1-(4-(methoxycarbonyl)-3-methylthiophen-2-yl)ethyl)piperidine-1-carboxylate

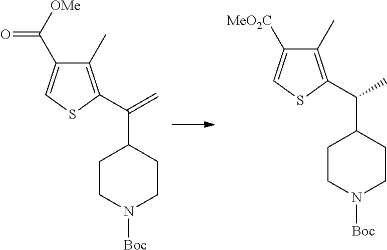 [SMILES: C=C(c1scc(C(=O)OC)c1C)C1CCN(C(=O)OC(C)(C)C)CC1 ; COC(=O)c1csc([C@H](C)C2CCN(C(=O)OC(C)(C)C)CC2)c1C]

A solution of tert-butyl 4-(1-(4-(methoxycarbonyl)-3-methylthiophen-2-yl)vinyl)piperidine-1-carboxylate (1.2 g, 3.28 mmol) and ((4R,5R)-(+)-O-[1-benzyl-1-(5-methyl-2-phenyl-4,5-dihydrooxazol-4-yl)-2-phenylethyl] (dicyclohexylphosphinite)(1,5-COD)iridium(I)tetrakis(3,5-bis(trifluoromethyl)phenylborate (63 mg, 36 μmol) in DCM (50 mL) was hydrogenated at 50 psi hydrogen pressure for 30 h on a Parr shaker. The mixture was concentrated and the residue was purified using column chromatography (silica gel, 0 to 40% EtOAc/hexanes) to give (R)-tert-Butyl 4-(1-(4-(methoxycarbonyl)-3-methylthiophen-2-yl)ethyl)piperidine-1-carboxylate (1.1 g) as a colorless oil. The optical purity of the product was determined to be 98% e.e. by chiral HPLC (Chiralpak AY-H, 5 microns, 4.6 mm×150 mm; 245, 250 nm UV; 90:10:0.1 n-heptane:EtOH: isopropylamine, isocratic, 1.0 ml/min). ¹H NMR (400 MHz, CDCl₃) δ 1.03-1.33 (m, 5H), 1.38-1.58 (m, 11H), 1.88 (d, J=12.38 Hz, 1H), 2.37 (s, 3H), 2.48-2.77 (m, 2H), 2.94 (quin, J=7.26 Hz, 1H), 3.85 (s, 3H), 4.05-4.15 (m, 1H), 7.97 (s, 1H). MS(ES) [M+H]⁺ 390.2.

### d) (R)-Methyl 5-(1-(1-(2,2-difluoroethyl)piperidin-4-yl)ethyl)-4-methylthiophene-3-carboxylate

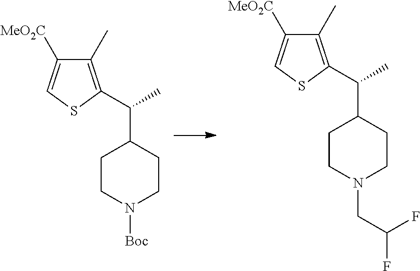 [SMILES: COC(=O)c1csc([C@H](C)C2CCN(C(=O)OC(C)(C)C)CC2)c1C ; COC(=O)c1csc([C@H](C)C2CCN(CC(F)F)CC2)c1C]

To a solution of (R)-tert-butyl 4-(1-(4-(methoxycarbonyl)-3-methylthiophen-2-yl)ethyl)piperidine-1-carboxylate (85 mg, 0.231 mmol) in DCM (2 mL) was added 4 N HCl in 1,4-dioxane (0.289 mL, 1.156 mmol). The mixture was maintained at RT for 4 h. The mixture was concentrated and the residue was dried under vacuum. The residue was then diluted with CH₃CN (2 mL). To this mixture were added 2,2-difluoroethyl trifluoromethanesulfonate (163 mg, 0.763 mmol) and Cs₂CO₃ (173 mg, 0.532 mmol). The mixture was heated at 50° C. for 2 h. The mixture was allowed to cool, diluted with DCM, and filtered. The filtrate was concentrated and the residue was purified using column chromatography (silica gel, 0 to 50% EtOAc/hexanes) to give (R)-methyl 5-(1-(1-(2,2-difluoroethyl)piperidin-4-yl)ethyl)-4-methylthiophene-3-carboxylate (68 mg) as a colorless oil.

### e) (R)-5-(1-(1-(2,2-Difluoroethyl)piperidin-4-yl)ethyl)-N-((4,6-dimethyl-2-oxo-1,2-dihydropyridin-3-yl)methyl)-4-methylthiophene-3-carboxamide

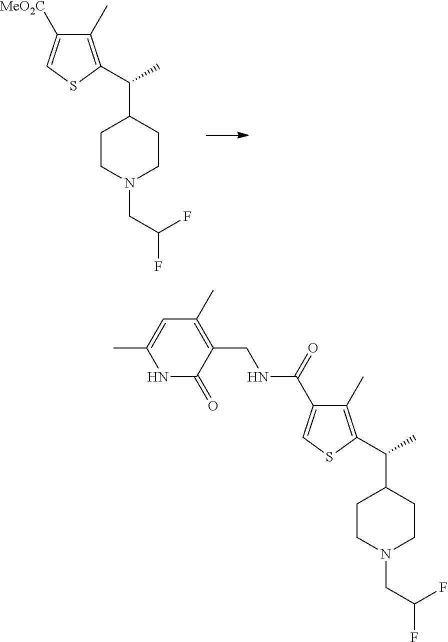 [SMILES: COC(=O)c1csc([C@H](C)C2CCN(CC(F)F)CC2)c1C ; Cc1cc(C)c(CNC(=O)c2csc([C@H](C)C3CCN(CC(F)F)CC3)c2C)c(=O)[nH]1]

To a solution of (R)-methyl 5-(1-(1-(2,2-difluoroethyl)piperidin-4-yl)ethyl)-4-methylthiophene-3-carboxylate (68 mg, 0.205 mmol) in MeOH (2 mL) was added 8 N NaOH (0.14 mL, 1.120 mmol). The mixture was heated at 40° C. for 18 h. The mixture was treated with 6 N HCl (0.187 mL, 1.12 mmol) and concentrated. The residue was dried under vacuum and diluted with DMSO (2 mL). To this mixture were added 3-(aminomethyl)-4,6-dimethylpyridin-2(1H)-one, hydrochloride (58.1 mg, 0.308 mmol), NMM (0.135 mL, 1.231 mmol), EDC (79 mg, 0.410 mmol), and HOAt (55.9 mg, 0.410 mmol). The mixture was stirred at RT for 18 h. The reaction mixture was quenched with water (10 mL) and the resulting precipitate was collected by filtration and dried under vacuum to give (R)-5-(1-(1-(2,2-difluoroethyl)piperidin-4-yl)ethyl)-N-((4,6-dimethyl-2-oxo-1,2-dihydropyridin-3-yl)methyl)-4-methylthiophene-3-carboxamide (49 mg) as an off-white solid. ¹H NMR (400 MHz, DMSO-d₆) δ 1.08-1.38 (m, 7H), 1.76 (m, 1H), 1.92-2.14 (m, 5H), 2.18 (m, 6H), 2.64 (td, J=15.73, 4.17 Hz, 2H), 2.82 (d, J=11.87 Hz, 1H), 2.86-2.97 (m, 2H), 4.17-4.30 (m, 2H), 5.86 (s, 1H), 5.94-6.23 (m, 1H), 7.65 (s, 1H), 7.97 (t, J=5.05 Hz, 1H). MS(ES) [M+H]⁺ 452.2.

### Example 23

### (R)-5-(1-(1-(2,2-Difluoropropyl)piperidin-4-yl)ethyl)-N-((4,6-dimethyl-2-oxo-1,2-dihydropyridin-3-yl)methyl)-4-methylthiophene-3-carboxamide

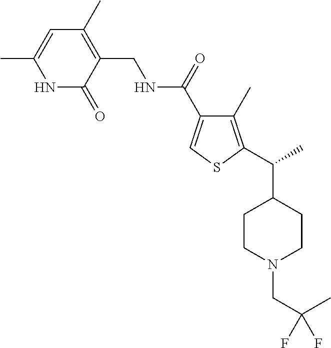 [SMILES: Cc1cc(C)c(CNC(=O)c2csc([C@H](C)C3CCN(CC(C)(F)F)CC3)c2C)c(=O)[nH]1]

Following the general procedure of Example 22, (R)-5-(1-(1-(2,2-difluoropropyl)piperidin-4-yl)ethyl)-N-((4,6-dimethyl-2-oxo-1,2-dihydropyridin-3-yl)methyl)-4-methylthiophene-3-carboxamide was prepared. ¹H NMR (400 MHz, DMSO-d₆) δ 1.09-1.39 (m, 7H), 1.58 (t, J=19.07 Hz, 3H), 1.76 (m, 1H), 1.98-2.27 (m, 12H), 2.63 (m, 2H), 2.81 (d, J=12.13 Hz, 1H), 2.91 (m, 2H), 4.17-4.28 (m, 2H), 5.86 (s, 1H), 7.65 (s, 1H), 7.97 (t, J=4.93 Hz, 1H). MS(ES) [M+H]⁺ 466.3.

### Assay Protocol

Compounds contained herein were evaluated for their ability to inhibit the methyltransferase activity of EZH2 within the PRC2 complex. Human PRC2 complex was prepared by co-expressing each of the 5 member proteins (FLAG-EZH2, EED, SUZ12, RbAp48, AEBP2) in Sf9 cells followed by co-purification. Enzyme activity was measured in a scintillation proximity assay (SPA) where a tritiated methyl group is transferred from 3H-SAM to a lysine residue on a biotinylated, unmethylated peptide substrate derived from histone H3. The peptides were captured on streptavidin-coated SPA beads and the resulting signal was read on a ViewLux plate reader.

### Part A. Compound Preparation

1. Prepare 10 mM stock of compounds from solid in 100% DMSO.
2. Set up an 11-point serial dilution (1:4 dilution, top concentration 10 mM) in 100% DMSO for each test compound in a 384 well plate leaving columns 6 and 18 for DMSO controls.
3. Dispense 10 nL of compound from the dilution plate into reaction plates (Corning, 384-well polystyrene NBS, Cat#3673).

### Part B. Reagent Preparation

Prepare the following solutions:
1. 1× Base Buffer, 50 mM Tris-HCl, pH 8, 2 mM MgCl₂: Per 1 L of base buffer, combine 1 M Tris-HCl, pH 8 (50 mL), 1 M MgCl₂ (2 mL), and distilled water (948 mL).
2. 1× Assay Buffer: Per 10 mL of 1× Assay Buffer, combine 1× Base Buffer (9.96 mL), 1 M DTT (40 uL), and 10% Tween-20 (1 uL) to provide a final concentration of 50 mM Tris-HCl, pH 8, 2 mM MgCl₂, 4 mM DTT, 0.001% Tween-20.
3. 2× Enzyme Solution: Per 10 mL of 2× Enzyme Solution, combine 1× Assay Buffer (9.99 mL) and 3.24 uM EZH2 5 member complex (6.17 uL) to provide a final enzyme concentration of 1 nM.
4. SPA Bead Solution: Per 1 mL of SPA Bead Solution, combine Streptavidin coated SPA beads (PerkinElmer, Cat# RPNQ0261, 40 mg) and 1× Assay Buffer (1 mL) to provide a working concentration of 40 mg/mL.
5. 2× Substrate Solution: Per 10 mL of 2× Substrate Solution, combine 40 mg/mL SPA Bead Solution (375 uL), 1 mM biotinylated histone H3K27 peptide (200 uL), 12.5 uM 3H-SAM (240 uL; 1 mCi/mL), 1 mM cold SAM (57 uL), and 1× Assay Buffer (9.13 mL) to provide a final concentration of 0.75 mg/mL SPA Bead Solution, 10 uM biotinylated histone H3K27 peptide, 0.15 uM 3H-SAM (˜12 uCi/mL 3H-SAM), and 2.85 uM cold SAM.
6. 2.67× Quench Solution: Per 10 mL of 2.67× Quench Solution, combine 1× Assay Buffer (9.73 mL) and 10 mM cold SAM (267 uL) to provide a final concentration of 100 uM cold SAM.

### Part C. Assay Reaction in 384-Well Grenier Bio-One Plates

### Compound Addition

1. Stamp 10 nL/well of 1000× Compound to test wells (as noted above).
2. Stamp 10 nL/well of 100% DMSO to columns 6 & 18 (high and low controls, respectively).

### Assay

1. Dispense 5 uL/well of 1× Assay Buffer to column 18 (low control reactions).
2. Dispense 5 uL/well of 2× Substrate Solution to columns 1-24 (note: substrate solution should be mixed to ensure homogeneous bead suspension before dispensing into matrix reservoir).
3. Dispense 5 uL/well of 2× Enzyme Solution to columns 1-17, 19-24.
4. Incubate the reaction for 60 min at room temperature.

### Quench

1. Dispense 6 uL/well of the 2.67× Quench Solution to columns 1-24.
2. Seal assay plates and spin for ˜1 min at 500 rpm.
3. Dark adapt plates in the ViewLux instrument for 15-60 min.

### Read Plates

1. Read the assay plates on the Viewlux Plate Reader utilizing the 613 nm emission filter or clear filter (300 s exposure).
   Reagent addition can be done manually or with automated liquid handler.

### Results

Percent inhibition was calculated relative to the DMSO control for each compound concentration and the resulting values were fit using standard IC₅₀ fitting parameters within the ABASE data fitting software package.

The exemplified compounds were generally tested according to the above or an analogous assay and were found to be inhibitors of EZH2. Specific biological activities tested according to such assays are listed in the following table. The IC₅₀ values of <10 nM indicate that the activity of compound was approaching the limit of detection in the assay. Repeating the assay run(s) may result in somewhat different IC₅₀ values.

| Example | EZH2 IC₅₀ (nM) |
|---|---|
| 1 | 16 |
| 2 | ≦10 |
| 3 | ≦10 |
| 4 | 20 |
| 5 | 501 |
| 6 | 50 |
| 7 | 20 |
| 8 | 16 |
| 9 | 50 |
| 10 | ≦10 |
| 11 | 20 |
| 12 | ≦10 |
| 13 | 13 |
| 14 | 40 |
| 15 | ≦10 |
| 16 | 100 |
| 17 | 100 |
| 18 | ≦10 |
| 19 | 316 |
| 20 | ≦10 |
| 21 | ≦10 |
| 22 | ≦10 |
| 23 | ≦10 |

1. A compound according to Formula (I) or a pharmaceutically acceptable salt thereof: 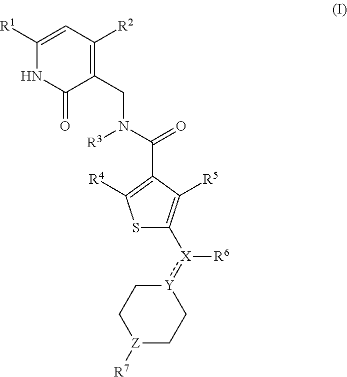
wherein:
X and Y are each independently CH, C, or N; wherein
when X is N, Y is CH, and [symbol] is a single bond;
when Y is N, X is CH, and [symbol] is a single bond;
when X and Y are each CH, [symbol] is a single bond; and
when X is C, Y is C, and [symbol] is a double bond;
Z is CH or N;
R¹ and R² are each independently (C₁-C₄)alkyl;
R³ and R⁴ are each hydrogen;
or R³ and R⁴ taken together represent —CH₂CH₂—;
R⁵ and R⁶ are each independently (C₁-C₃)alkyl; and
R⁷ is selected from the group consisting of halo(C₁-C₄)alkyl, —N((C₁-C₄)alkyl)₂, hydroxyl, pyrimidinyl, oxazolylmethyl, and —C(═N—CN)NH(C₁-C₄)alkyl;
provided that the compound is not N-((4,6-dimethyl-2-oxo-1,2-dihydropyridin-3-yl)methyl)-5-((4-(dimethylamino)cyclohexyl)(ethyl)amino)-4-methylthiophene-3-carboxamide, 5-((4-(dimethylamino)cyclohexyl)(ethyl)amino)-4-methyl-N-((6-methyl-2-oxo-4-propyl-1,2-dihydropyridin-3-yl)methyl)thiophene-3-carboxamide, N-((4,6-dimethyl-2-oxo-1,2-dihydropyridin-3-yl)methyl)-5-(1-(4-(dimethylamino)piperidin-1-yl)ethyl)-4-methylthiophene-3-carboxamide, N-((4,6-dimethyl-2-oxo-1,2-dihydropyridin-3-yl)methyl)-5-(1-(4-(dimethylamino)piperidin-1-yl)propyl)-4-methylthiophene-3-carboxamide, N-((4,6-dimethyl-2-oxo-1,2-dihydropyridin-3-yl)methyl)-5-(1-(4-(dimethylamino)cyclohexyl)propyl)-4-methylthiophene-3-carboxamide, or N-((4,6-dimethyl-2-oxo-1,2-dihydropyridin-3-yl)methyl)-5-(ethyl(4-(ethyl(methyl)amino)cyclohexyl)amino)-4-methylthiophene-3-carboxamide, or stereoisomers or mixtures thereof of each of these compounds.

2. The compound or pharmaceutically acceptable salt thereof according to claim 1, wherein X and Y are each independently CH or N, wherein at least one of X and Y is CH and [symbol] is a single bond.

3. The compound or pharmaceutically acceptable salt thereof according to claim 1, wherein X is N, Y is CH, and [symbol] is a single bond.

4. The compound or pharmaceutically acceptable salt thereof according to claim 1, wherein Y is N, X is CH, and [symbol] is a single bond.

5. The compound or pharmaceutically acceptable salt thereof according to claim 1, wherein X and Y are each CH and [symbol] is a single bond.

6. The compound or pharmaceutically acceptable salt thereof according to claim 1, wherein X and Y are each C and [symbol] is a double bond.

7. The compound or pharmaceutically acceptable salt thereof according to claim 1, wherein Z is CH.

8. The compound or pharmaceutically acceptable salt thereof according to claim 1, wherein Z is N.

9. The compound or pharmaceutically acceptable salt thereof according to claim 1, wherein R¹ and R² are each methyl.

10. The compound or pharmaceutically acceptable salt thereof according to claim 1, wherein R³ and R⁴ are each hydrogen.

11. The compound or pharmaceutically acceptable salt thereof according to claim 1, wherein R⁵ is methyl.

12. The compound or pharmaceutically acceptable salt thereof according to claim 1, wherein R⁶ is ethyl.

13. The compound or pharmaceutically acceptable salt thereof according to claim 1, wherein R⁷ is selected from the group consisting of halo(C₁-C₄)alkyl, —N((C₁-C₄)alkyl)₂, and hydroxyl.

14. The compound according to claim 1 which is:
5-(1-(1-(2,2-difluoropropyl)piperidin-4-ylidene)propyl)-N-((4,6-dimethyl-2-oxo-1,2-dihydropyridin-3-yl)methyl)-4-methylthiophene-3-carboxamide;
N-((4,6-dimethyl-2-oxo-1,2-dihydropyridin-3-yl)methyl)-5-(1-(1-(2-fluoro-2-methylpropyl)piperidin-4-ylidene)propyl)-4-methylthiophene-3-carboxamide;
N-((4,6-dimethyl-2-oxo-1,2-dihydropyridin-3-yl)methyl)-4-methyl-5-(1-(1-(2,2,2-trifluoroethyl)piperidin-4-ylidene)propyl)thiophene-3-carboxamide;
(R)-5-(1-(1-(2,2-difluoropropyl)piperidin-4-yl)propyl)-N-((4,6-dimethyl-2-oxo-1,2-dihydropyridin-3-yl)methyl)-4-methylthiophene-3-carboxamide;
(S)-5-(1-(1-(2,2-difluoropropyl)piperidin-4-yl)propyl)-N-((4,6-dimethyl-2-oxo-1,2-dihydropyridin-3-yl)methyl)-4-methylthiophene-3-carboxamide;
N-((4,6-dimethyl-2-oxo-1,2-dihydropyridin-3-yl)methyl)-5-(1-(1-(2-fluoro-2-methylpropyl)piperidin-4-yl)propyl)-4-methylthiophene-3-carboxamide;
N-((4,6-dimethyl-2-oxo-1,2-dihydropyridin-3-yl)methyl)-5-(1-(1-(dimethylamino)piperidin-4-ylidene)propyl)-4-methylthiophene-3-carboxamide;
N-((4,6-dimethyl-2-oxo-1,2-dihydropyridin-3-yl)methyl)-5-(1-(1-hydroxypiperidin-4-ylidene)propyl)-4-methylthiophene-3-carboxamide;
5-((4,6-dimethyl-2-oxo-1,2-dihydropyridin-3-yl)methyl)-2-(1-(4-(dimethylamino)piperidin-1-yl)propyl)-3-methyl-6,7-dihydrothieno[3,2-c]pyridin-4(5H)-one;
N-((4,6-dimethyl-2-oxo-1,2-dihydropyridin-3-yl)methyl)-5-(1-(4-hydroxycyclohexylidene)propyl)-4-methylthiophene-3-carboxamide;
N-((4,6-dimethyl-2-oxo-1,2-dihydropyridin-3-yl)methyl)-4-methyl-5-(1-(1-(2,2,2-trifluoroethyl)piperidin-4-yl)propyl)thiophene-3-carboxamide;
N-((4,6-dimethyl-2-oxo-1,2-dihydropyridin-3-yl)methyl)-4-methyl-5-(1-(1-(oxazol-2-ylmethyl)piperidin-4-ylidene)propyl)thiophene-3-carboxamide;
N-((4,6-dimethyl-2-oxo-1,2-dihydropyridin-3-yl)methyl)-4-methyl-5-(1-(1-(pyrimidin-2-yl)piperidin-4-ylidene)propyl)thiophene-3-carboxamide;
5-(1-(1-(2,2-difluoroethyl)piperidin-4-yl)propyl)-N-((4,6-dimethyl-2-oxo-1,2-dihydropyridin-3-yl)methyl)-4-methylthiophene-3-carboxamide;
5-(1-(1-(N′-cyano-N-methylcarbamimidoyl)piperidin-4-ylidene)propyl)-N-((4,6-dimethyl-2-oxo-1,2-dihydropyridin-3-yl)methyl)-4-methylthiophene-3-carboxamide;
2-(1-(1-(2,2-difluoropropyl)piperidin-4-yl)propyl)-5-((4,6-dimethyl-2-oxo-1,2-dihydropyridin-3-yl)methyl)-3-methyl-6,7-dihydrothieno[3,2-c]pyridin-4(5H)-one;
5-((4,6-dimethyl-2-oxo-1,2-dihydropyridin-3-yl)methyl)-2-(1-(1-(2-fluoropropyl)piperidin-4-yl)propyl)-3-methyl-6,7-dihydrothieno[3,2-c]pyridin-4(5H)-one;
N′-cyano-4-(1-(5-((4,6-dimethyl-2-oxo-1,2-dihydropyridin-3-yl)methyl)-3-methyl-4-oxo-4,5,6,7-tetrahydrothieno[3,2-c]pyridin-2-yl)propyl)-N-methylpiperidine-1-carboximidamide;
5-(1-(1-(N′-cyano-N-methylcarbamimidoyl)piperidin-4-yl)propyl)-N-((4,6-dimethyl-2-oxo-1,2-dihydropyridin-3-yl)methyl)-4-methylthiophene-3-carboxamide;
5-(1-(1-(2,2-difluoroethyl)piperidin-4-ylidene)propyl)-N-((4,6-dimethyl-2-oxo-1,2-dihydropyridin-3-yl)methyl)-4-methylthiophene-3-carboxamide;
(R)-5-(1-(1-(2,2-difluoroethyl)piperidin-4-yl)propyl)-N-((4, 6-dimethyl-2-oxo-1,2-dihydropyridin-3-yl)methyl)-4-methylthiophene-3-carboxamide;
(R)-5-(1-(1-(2,2-difluoroethyl)piperidin-4-yl)ethyl)-N-((4,6-dimethyl-2-oxo-1,2-dihydropyridin-3-yl)methyl)-4-methylthiophene-3-carboxamide; or
(R)-5-(1-(1-(2,2-difluoropropyl)piperidin-4-yl)ethyl)-N-((4,6-dimethyl-2-oxo-1,2-dihydropyridin-3-yl)methyl)-4-methylthiophene-3-carboxamide;
or a pharmaceutically acceptable salt thereof.

15. A pharmaceutical composition comprising the compound or pharmaceutically acceptable salt thereof according to claim 1 and a pharmaceutically acceptable excipient.

16. A method of treating cancer comprising administering to a patient with cancer a therapeutically effective amount of the compound or pharmaceutically acceptable salt thereof according to claim 1.

17. The method of claim 16, wherein said cancer is selected from the group consisting of: brain (gliomas), glioblastomas, leukemias, lymphomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, gastric, bladder, head and neck, kidney, lung, liver, melanoma, renal, ovarian, pancreatic, prostate, sarcoma, osteosarcoma, giant cell tumor of bone, and thyroid.

18. (canceled)
